(19) United States
(12) Patent Application Publication
(10) Pub. No.: US 2026/0071965 A1
(43) Pub. Date: Mar. 12, 2026

(54) SENSORS USING LIQUID METAL-BASED NANOPHOTONIC STRUCTURES

(71) Applicant: The Research Foundation for The State University of New York, Amherst, NY (US)

(72) Inventors: Qiang LIU, Amherst, NY (US); Xianglong MIAO, Amherst, NY (US); Shreyan DATTA, Amherst, NY (US)

(21) Appl. No.: 19/109,520

(22) PCT Filed: Sep. 7, 2023
(86) PCT No.: PCT/US2023/073691

§ 371 Date: Mar. 6, 2025

Related U.S. Application Data
(60) Provisional application No. 63/374,901, filed on Sep. 7, 2022.

(51) Int. Cl. G01N 21/65; B82Y 20/00; B82Y 40/00; G01J 3/44

(52) U.S. Cl. CPC G01N 21/658; B82Y 20/00; B82Y 40/00; G01J 3/4412; G01N 2021/651

(57) ABSTRACT

Methods of preparing a sample for Raman spectroscopy and sensor for Raman spectroscopy are disclosed. A method includes disposing a plurality of metal nanoparticles on a first surface of a substrate: depositing an analyte of interest on the metal nanoparticles; and coating at least a portion of the first surface of the substrate in a liquid metal such that the plurality of metal nanoparticles and analyte of interest are encapsulated between the first surface of the substrate and the liquid metal.

### CROSS-REFERENCE TO RELATED APPLICATIONS

This application claims priority to U.S. Provisional Application No. 63/374,901, filed on Sep. 7, 2022, now pending, the disclosure of which is incorporated herein by reference.

### STATEMENT REGARDING FEDERALLY SPONSORED RESEARCH

This invention was made with government support under contract no. 1847203 awarded by the National Science Foundation. The government has certain rights in the invention.

### FIELD OF THE DISCLOSURE

The present disclosure relates to molecular sensors, and more particularly to sensors for Raman spectroscopy.

### BACKGROUND OF THE DISCLOSURE

Optical sensors achieve sensitive and selective detection of trace analytes by producing measurable optical signals which are determined by the interactions between the probing light and the probed analytes, e.g., via Raman scattering. To enhance the light-analyte interactions and thus the optical sensing signals, optical sensors often employ nanoscale photonic structures (usually based on solid metals and dielectrics) which can confine light and enhance its intensity in tiny nanoscale volumes at certain locations of the device surface, which are referred to as “hot spots.” Analytes in the hot spots of such nanophotonic sensors will experience drastically enhanced interactions with the incident light, and therefore produce much more optical sensing signals. In general, the smaller the hot spots, the stronger the light-analyte interactions. However, the major drawbacks of conventional nanophotonic sensors are (1) it is difficult and costly to fabricate nanophotonic structures with nanoscale hot spots; and (2) it is usually very difficult and time-consuming for the analytes to enter the nanoscale hot spots (e.g., through random diffusion process in a liquid medium).

Resonant nanophotonic structures excited by incident light can confine highly enhanced electric field in deep-subwavelength regions (hot spots) where the interactions between light and analyte molecules can be drastically enhanced, leading to significant improvement of the sensing performance. Designing nanophotonic structures with smaller hot spots is a widely utilized strategy to increase field enhancement and improve sensor performance. A variety of nanophotonic structures with nanometric gaps have been demonstrated for sensing applications, such as dimer antennas, split-ring resonators, coaxial disk resonators, and nano-patch antennas. However, with the gap size decreasing down to the nanometric scale, it becomes increasingly difficult to deliver analyte molecules into these gaps (i.e., the hot spots), especially when the gap size is comparable to typical sizes of molecules. This issue fundamentally limits the further performance improvement of nanophotonic sensors. An effective approach to addressing this issue is to deliver the analytes before forming the nanometric gaps (or other types of hot spot structures). For example, metallic nanoparticles coated with analyte thin films can form super-crystals with nanometric separation gaps. In a recent study, such super-crystals of gold nanoparticles coated with thiolated polystyrene molecules were demonstrated to function as SEIRA sensors for sensing the polystyrene molecules with high performance. Another previous demonstration of SEIRA sensors based on graphene acoustic plasmon resonators realized effective delivery of analytes into nanometric gaps by first spin-coating a thin analyte film on gold nanoribbons and subsequently transferring graphene onto the analyte film, which led to the successful detection of SEIRA signals from sub-nm thick analyte films. Nevertheless, assembling super-crystals of metal nanoparticles or transferring graphene to form the complete sensor structures is not a simple and straightforward process, and hence may not be suitable for point-of-care applications.

### BRIEF SUMMARY OF THE DISCLOSURE

To overcome the drawbacks of conventional nanophotonic sensors, the present disclosure provides a new nanophotonic sensor architecture which employs liquid-metal-based nanophotonic structures to form nanoscale hot spots. This innovative device architecture allows the analyte to be delivered in place before the nanoscale hot spots are formed by the liquid metal, hence achieving not only hot spots with high light confinement and enhancement, but also highly efficient delivery of analytes into the hot spots. These benefits result in significantly improved sensing performance. Furthermore, the present disclosure provides an efficient device fabrication process that does not require photolithography or electron beam lithography, and thus making the demonstrated liquid-metal-based nanophotonic sensors a potentially more cost-effective technology than the conventional nanophotonic sensors with similar functionalities.

The presently-disclosed liquid-metal nanophotonic sensors employ solid metal nanostructures (such as nanoparticles) implemented on a suitable substrate (such as a thin slab of glass) to form the sensor chip. These solid metal nanostructures can be formed by depositing a thin metal film (e.g., gold film) of a few nm on the substrate and then annealing the sample at a high temperature (e.g., 800 degrees Celsius) for a few minutes (see FIG. 11 for an example). These solid metal nanostructures can also be realized using a standard nanofabrication process involving lithography, metal deposition and lift-off. After the sensor chip is ready, the target analytes in a liquid medium can be introduced onto the sensor chip using a pipette or other tools with similar function. The sensor chip can also be functioned with bio-recognition elements such as specific antibodies to selectively capture specific biomarkers. After the liquid medium containing the analytes evaporates, the target analytes are adsorbed on the sensor chip surface, coating the solid metal nanostructures. The sensor chip is then placed on a small amount of liquid metal (such as liquid gallium or liquid gallium-indium eutectic) in a smaller container, with the analyte-coated chip surface facing the liquid metal. In this way, the complete structure of the presently-disclosed liquid-metal nanophotonic sensor is formed, and the target analytes are tightly sandwiched by the liquid metal and the solid metal nanostructures, which constitute nanoscale cavities with exceedingly high light confinement and enhancement to enhance the interactions between the incident light and the target analytes in these nanoscale cavities (i.e., hot spots). The target analytes can be probed by Raman spectroscopy with an incident laser illuminating the sample from the substrate side (e.g., glass) and the Raman scattering signal collected from the same side. FIG. 12 illustrates the structure of an example device. As the analytes are present in the nanoscale cavities formed by the liquid metal and the solid metal nanostructures on the sensor chip, the corresponding Raman scattering signals due to the analytes are enhanced by several orders of magnitude, when compared to having the analytes on a blank substrate (i.e., without any solid metal nanostructures or liquid metal). Furthermore, compared to the Raman scattering signals obtained from the analytes on the sensor chips alone (i.e., without placing the sensor chips on the liquid metal), the Raman scattering signals obtained with the sensor chips placed on the liquid metal are enhanced by several times, clearly demonstrating the advantages of our liquid-metal nanophotonic sensor architecture (see FIGS. 13 and 14).

### DESCRIPTION OF THE DRAWINGS

For a fuller understanding of the nature and objects of the disclosure, reference should be made to the following detailed description taken in conjunction with the accompanying drawings

FIG. 1: Schematic figure of an example sensor according to an embodiment of the present disclosure.

FIG. 2: Diagram of an example method of making a sensor according to an embodiment of the present disclosure.

FIG. 3: (a) Photograph of the sputter coater used to create experimental embodiments of the present sensor; (b) An SEM image of a deposited gold thin film used in an experimental embodiment.

FIG. 4: Schematics of the structures in the simulation model. The top row represents an individual gold nanosphere, whereas the bottom row represents a nanosphere dimer.

FIG. 5: Numerical simulation results of sphere nanoparticles with different diameters. (a) Reflection spectrum of (a) nanosphere and (b) nanosphere coated with liquid gallium. Electric field enhancement distribution for (c) nanosphere and (d) nanosphere coated with gallium.

FIG. 6: Numerical simulation results of sphere dimer with different diameters. (a) Reflection spectrum of (a) dimer and (b) dimer coated with liquid gallium. Electric field enhancement distribution for (c) dimer and (d) dimer coated with gallium.

FIG. 7: SERS spectra of sensor chip N1. (a) Particle size distribution of sensor chip. The inset shows the SEM image of sensor surface. (b) Measured Raman spectra of device before and after coating with liquid gallium. (c) Extracted Raman peaks spectra. (d) and (e) 2D mapping of the Raman signals at 1080 cm⁻¹ before (d) and after (e) coating the liquid gallium, respectively.

FIG. 8: SERS spectra of sensor chip N2. (a) Particle size distribution of sensor chip. The inset shows the SEM image of sensor surface. (b) Measured Raman spectra of device before and after coating with liquid gallium. (c) Extracted Raman peaks spectra. (d) and (e) 2D mapping of the Raman signals at 1080 cm⁻¹ before (d) and after (e) coating the liquid gallium, respectively.

FIG. 9: SERS spectra of sensor chips prepared under the annealing temperature (a) 700° C., (b) 800° C., (c) 900° C., (d) 1000° C. The insets show the SEM image of the corresponding sensor surface.

FIG. 10: SERS spectra of sensor chip N7. (a) Particle size distribution of sensor chip. The inset shows the SEM image of sensor surface. (b) Measured Raman spectra of sensor before and after coating with liquid gallium. (c) Extracted Raman peaks spectra.

FIG. 11: Scanning electron microscope image of gold nanoparticles distributed on a sensor chip surface. The gold nanoparticles were formed by depositing a few nm of gold thin film on the glass substrate and then annealing the sample at a high temperature (e.g., 800 degrees Celsius).

FIG. 12: Schematic illustrating an example liquid-metal nanophotonic sensor for surface-enhanced Raman scattering (SERS) sensing of analytes which are tightly sandwiched between gold nanoparticles and liquid metal.

FIG. 13: Comparison between the Raman scattering spectrum of an analyte obtained by an example nanophotonic sensor with a liquid-gallium-based device structure according to an embodiment of the present disclosure (upper curve) and that without the liquid gallium (bottom curve).

FIG. 14: Comparison between the intensity of a Raman peak of the analyte obtained across a large region of an example nanophotonic sensor with the liquid-gallium-based device structure according to an embodiment of the present disclosure (bottom figure) and the intensity of the same Raman peak of the analyte obtained across a large region of the nanophotonic sensor without the liquid gallium (top figure).

FIG. 15 15: Types of the vibrational modes with the water molecule as an example

FIG. 16: Infrared and Raman spectrum of styrene-butadiene rubber

FIG. 17: IR absorption of vibrational modes in CO₂

FIG. 18: The typical configuration of the interferometer in the FTIR system

FIG. 19: The schematic of the Raman spectroscopy

FIG. 20: The optical extinction spectrum and the electric field enhancement of the gold nanoparticles.

FIG. 21: Absorption profile depends on the q.

FIG. 22: Schematic of tip-enhanced Raman spectroscopy

FIG. 23: (a) Structure of the MIM resonator. (b) the vertical interference of MIM resonator. (c). the horizontal resonant cavity of nano-patch antenna

FIG. 24: (a) Schematic of the hybrid structure. (b) Illustration of the design rationale of the hybrid structures. (c) Near-field-intensity distribution corresponding to the structures in part b: from the left to right are the gold disk, graphene antidot, and hybrid structure, respectively.

FIG. 25: Fabrication of hybrid anti-dot device. (a) Schematic of the key steps of the device fabrication process for realizing self-aligned gold disks in graphene anti-dots. (b) SEM images of the fabricated device. The right figure is a zoomed part for the left one. The gap size is around 70 120 nm. The scale bar in the image is 500 nm. (c) the fabrication process of the device.

FIG. 26: Characterization of the transferred graphene. (a) optical image with 100× objective. (b) Raman spectroscopy of graphene with 532 nm laser excitation.

FIG. 27: Characterization of the transferred graphene. (a) optical image with 100× objective. (b) Raman spectroscopy of graphene with 532 nm laser excitation.

FIG. 28: Transfer curve of a fabricated device

FIG. 29: Approximate circuit model for a graphene anti-dot unit cell (4, 2).

FIG. 30: Fitting of graphene channel resistance. Solid curve is the measured total resistance dependence on gate voltage. Symbols are the fitting result using the aforementioned method.

FIG. 31: Simulated transmission extinction spectra of various hybrid antidot structures in comparison with those of the corresponding bare antidot structures: (a) structures with index (3, 2); (b) structures with index (4, 2); (c) structures with index (5, 3). Solid lines are for the hybrid antidot structures, and dashed lines are for the bare antidot structures.

FIG. 32: (a) Transmission extinction spectrum of the hybrid antidot metasurface (3, 2) in comparison with that of the bare graphene antidot metasurface (3, 2), assuming a carrier scattering rate of 1 meV. The different modes are labeled by the numbers 1-3 and 1′-3′. (b-d) Electric-field-intensity distributions of modes 1-3 of the bare graphene antidot metasurface in part a, respectively. (e-g) Electric-field-intensity distributions of modes 1′-3′ of the hybrid antidot metasurface in part (a), respectively.

FIG. 33: (a). Simulated transmission extinction spectra of the hybrid structure (4, 2) with various gap sizes. (b). Field enhancement of the hybrid structure (4, 2). The Fermi level of graphene is set to 0.5 eV, and the carrier scattering rate is 5 meV.

FIG. 34: Optical response of gold disk array. (a) Schematic of a gold disk under polarized excitation. The green line marks the position of line monitor. (b) Simulated near-field intensity along the line monitor in (a) versus frequency. (c) Experimentally measured transmission spectrum of a gold disk array (3, 2) in the terahertz to mid-infrared range.

FIG. 35: Experimentally measured transmission extinction spectra for different devices of the specified geometries (4,2) and (5,3). The red arrows indicate different resonant modes of each device.

FIG. 36: (a)-(c) Experimentally measured transmission extinction spectra for different devices of the specified geometries (index numbers). The red arrows indicate different resonant modes of each device. (d) Extracted frequencies of the resonant GSP modes of different devices versus the graphene carrier density n (plotted as n^{1/4}). The symbols are the extracted experimental values. The solid lines are fit to the experimental data using the formula ω=A·n^{1/4}, where A is a fitting parameter.

FIG. 37: (a) Schematic of the sensor structure. The parameters w, d and L represent the width of Al ribbons, the height of Ge ribbons and the width of nano-trenches, respectively. (b) Simulated electric near field distribution of an exemplary resonator design with d=200 nm, w=1.4 mum, and L=400 nm. (c) Reflection spectrum (red curve) of the resonators in (b) with the nano-trenches filled with an “imaginary” molecular species which has a single absorption line at ˜1550 cm⁻¹ (black curve).

FIG. 38: Schematics depicting the underlying process of the molecule trapping functionality of the nano-trench structures.

FIG. 39: (a) Schematics of the device fabrication process. (b) SEM images of the fabricated devices taken at an electron accelerating voltage of 20 kV. The left and upper-right images are top views of a device structure. The lower-right image is the cross-sectional view of a resonator structure cut using FIB. The scale bar in the left image is 2 μm and the scale bars in the right two images are 600 nm.

FIG. 40: Numerical simulation results. (a) Top: reflection spectra of resonator designs with the Al ribbon width ranging from 800 nm to 1.4 μm. The nano-trench width is 200 nm. Bottom: reflection spectra of resonator designs with the nano-trench width ranging from 0 to 300 nm. The Al ribbon width is 1 μm. The black curve corresponds to the proline absorption spectrum, and the shaded regions mark the main proline absorption lines. (b) Reflection spectra of three resonator designs with the specified Al ribbon widths and a fixed nano-trench width of 400 nm, before (dashed curves) and after (solid curves) the inner side of the nano-trenches is filled with 100 nm wide proline. The dot-dashed curves are the differential reflection spectra of these resonators due to the addition of proline.

FIG. 41: (a) Reflection spectra of three resonator designs with the specified nano-trench widths, with 100 nm wide proline filling either the inner side (solid curves) or the outer side (dashed curves) of the nano-trenches. (b) Extracted differential reflection spectra of the resonator designs in c due to proline filling the inner sider (top) and outer side (bottom) of the nano-trenches. The insets show where the Profile is added. Note that for the design with L=100 nm, the 100 nm wide proline fills the nano-trenches completely, so there is no distinction between the inner side and the outer side in this case. (c) and (d) Simulated electric field distributions of the resonator designs in (a) at the frequencies of two proline absorption lines, i.e., 1453 cm⁻¹ and 1630 cm⁻¹, respectively. The blue solid lines represent the Ge ribbons.

FIG. 42: Optical microscope images of (a) the bare device, (b) the device with L-proline precipitate, (c) the device with D-glucose precipitate, and (d) the device with sodium chloride precipitate. The scale bars in (c)-(f) are all 5 μm

FIG. 43: (a) Schematic of the modified device structure used for liposomes trapping and fluorescence imaging. (b) Reflection image of the device structure using an illumination at 488 nm wavelength. The Photoresist ribbons, the underlying Ge ribbons and the nano-trenches were clearly observed. (c) Fluorescence image of the same device area in (b). The excitation wavelength was 640 nm, and the emission wavelength was 707 nm.

FIG. 44: L-proline sensing results. (a) and (b) Reflection spectra of two sets of devices with (a) 200 nm wide nano-trenches, and (b) 450 nm wide nano-trenches, before (dot-dashed curves) and after (solid curves) introducing a 1 μL droplet of 10 μg/mL proline solution, respectively. (c) Reflection spectra of two devices with 450 nm wide nano-trenches, before (dot-dashed curves) and after (solid curves) introducing a 1 μL droplet of 1 μg/mL proline solution. (d) Upper panel: reflection spectra of the device with 450 nm wide nano-trenches and 1.4 μm wide Al ribbons, before (dot-dashed black curve) and after (solid red curve) introducing a 1 μL droplet of 0.2 μg/mL proline solution. Lower panel: the differential reflection spectra extracted from the spectra in the upper panel. The red (blue) curve is the differential spectrum before (after) the procedure for removing/reducing the interfering spectral features due to water vapor absorption lines

FIG. 45: D-glucose sensing results. (a) Reflection spectrum of a device before (dot-dashed curves) and after (solid curves) introducing a 1 μL droplet of glucose solution at three different concentrations. The black solid curve corresponds to the glucose absorption spectrum. (b) Extracted differential spectra from the data of the two lowest concentrations in (a), both before and after applying the correction for the interfering spectral features due to the water absorption lines.

FIG. 46: (a) Ten sequential and independent measurements of the reflection spectrum of a bare device using the same settings as the sensing experiments. (b) Calculated standard deviation of the reflection spectra plotted in (a).

FIG. 47: Reliability test of L-proline sensing. (a) and (b) Reflection spectra of repeated sensing experiments for 1 μg/mL proline solution by using a sensor with 200 nm wide nano-trenches in (a), and a sensor with 450 nm wide nano-trenches in (b). The black dot-dashed curve in each graph is the reflection spectra of the bare device. (c) Reflection spectra measured at the 4 quadrants of the sensor in (b) after two 1 μL droplets of 0.2 μg/mL proline solution are applied. Each quadrant (A, B, C or D) has an area of approximately 150 μm by 150 μm. (d) Representative SEM images showing the non-uniform proline precipitate distribution in quadrant B (top) and quadrant D (bottom). The SEM images are taken at a view angle 45° of the device surface normal. The structures and color changes near the lower edge of the Al ribbon in each image corresponds to the proline precipitate. The scale bar is 400 nm.

FIG. 48: Structure of liquid-gallium-based SEIRA sensors. (a) Schematic of the sensor structure. (b) Measured spectra of the ellipsometry parameters Ψ and Δ for a flat liquid gallium surface, where Ψ and Δ are related to the complex reflectance ratio as rₚ/rₛ=tan Ψ eiΔ, rₚ and rₛ are the complex reflectance for the p-polarization and s-polarization, respectively. (c) The real part (blue line) and imaginary part (red line) of the relative permittivity function of liquid gallium calculated from the ellipsometry measurement results in (b). (d) Simulated distributions of the electric field intensity (left panel), the amplitude of the x-component of the electric field (middle panel), and the amplitude of the y-component of the electric field (right panel), at the resonance of the nano-patch antenna employing a liquid gallium ground plane. These field distributions are all normalized to the incident field.

FIG. 49: Sensor device fabrication process

FIG. 50: Schematics of the procedure for constructing liquid-gallium-based SEIRA sensors for sensing nanometric analyte films. An SEM image of the fabricated gold nanostrips, a photo of a small quantity of liquid gallium squeezed out of a syringe and onto a glass slide, and an optical image of a sensor chip observed under the microscope are also shown in the figure.

FIG. 51: (a) Relative permittivity of ODT extracted from the measured reflection spectrum of SAM ODT on a gold surface. The purple and green shaded regions mark the CH₂- and CH₃-vibrational modes. The inset gives a schematic of the ODT molecule. (b) Schematic of a nano-patch antenna with the SAM ODT sandwiched between the gold nanostrip and the liquid gallium ground plane.

FIG. 52: Experimental results of sensing SAM ODT. (a) Measured reflection spectra of liquid-gallium-based sensors with SAM ODT. The yellow shaded region highlights the spectral range containing the ODT molecular vibrational modes. The absorption lines near 2400 cm⁻¹ are due to CO2 absorption in the optical path of our experimental setup. The spectra plotted in (a) are stacked vertically with 25% offset between neighboring curves. (b) Extracted net molecular vibrational signals associated with the SAM ODT from (a). The spectra plotted in (b) are stacked vertically with 10% offset between neighboring curves.

FIG. 53: (a) Simulated reflection spectra of a liquid-gallium-based nano-patch antenna structure (200 nm wide gold nanostrip) for two orthogonal linear polarizations of the incident light, i.e., polarized perpendicular to the gold nanostrip (red curve), and polarized parallel to the gold nanostrip (blue curve). It is clear that the nano-patch antenna structure has no resonance in the simulated spectral region when the incident light is polarized parallel to the gold nanostrip. (b)-(c) Measured reflection spectra of another set of liquid-gallium-based nano-patch antenna sensors targeting SAM ODT, with the incident light polarized (b) perpendicular to the gold nanostrips and (c) parallel to the gold nanostrips. The spectra plotted in (b) and (c) are stacked vertically with 25% offset between neighboring curves. As expected, strong SEIRA signals associated with the SAM ODT were observed when the incident light was polarized perpendicular to the gold nanostrips (similar to the results presented in the main text). In contrast, no SEIRA signal associated with the SAM ODT was observed when the incident light was polarized parallel to the gold nanostrips.

FIG. 54: (a) SEM images of a sensor surface after the liquid gallium was removed. The scale bars are both 2 μm. (b) Measured EDS spectrum of the gold nanostrip surface after the liquid gallium was removed. The electron gun accelerating voltage was 20 kV. The inset is an SEM image of the device surface and the EDS measurement region is marked by the pink rectangle. The gallium peak (Kα-9.241 keV) is not observed in the EDS spectrum. (c) Measured reflection spectra of the sensors with a gold ground plane directly deposited on the SAM ODT using electron-beam evaporation. The spectra plotted in (c) are stacked vertically with 10% offset between neighboring curves.

FIG. 55: Measured reflection spectra of nano-patch antennas coated with SAM ODT and with a thin aluminum oxide layer deposited using atomic layer deposition on the SAM ODT, followed by a gold film (i.e., the ground plane) deposited on the aluminum oxide layer using an electron beam evaporator. As explained in the main text, the aluminum oxide layer is added to protect the SAM ODT from the deposition of the gold ground plane. The aluminum oxide layer thickness is approximately 2 nm for the devices in (a), approximately 3 nm for the devices in (c), and approximately 5 nm for the devices in (e). The spectra plotted in (a), (c) and (e) are stacked vertically with 25% offset between neighboring curves. (b), (d) and (f) show the extracted net molecular vibrational signals associated with the SAM ODT from (a), (c) and (e), respectively. The spectra plotted in (b), (d) and (f) are stacked vertically with 10% offset between neighboring curves. In addition, the black bar in (b), (d) and (f) represents the y-axis scale: it has a length that corresponds to 10%. The yellow region in these figures marks the spectral range containing the ODT vibrational modes, whereas the grey region marks the absorption lines due to the carbon dioxide in the air (which is not a sensing signal produced by our sensors).

FIG. 56: Measured reflection spectra of the liquid-gallium-based sensors with (a) a 10 nm thick PMMA film, (b) a 32 nm thick PMMA film, and (c) a 67 nm thick PMMA film. The thin black line in each figure represents the imaginary part of the PMMA refractive index. The spectra plotted in (a) and (b) are stacked vertically with 20% offset between neighboring curves. The spectra plotted in (c) are stacked vertically with 40% offset between neighboring curves.

FIG. 57: Schematic of the equivalent local model used in our simulations to take into account the non-local effect of the metals (i.e., Au and liquid Ga).

FIG. 58: Simulation results of the liquid-gallium-based sensors. (a) Simulated reflection spectra of liquid-gallium-based sensors with a 2.4 nm thick (monolayer) ODT in the gaps of the nano-patch antennas. The spectra plotted in (a) are stacked vertically with 25% offset between neighboring curves. (b) Measured and simulated resonant frequencies of the nano-patch antennas versus the inverse of gold nanostrip width. (c) Simulated reflection spectra of the liquid-gallium-based nano-patch antennas with different gap sizes. In the corresponding simulation models, the nanometric gaps of the nano-patch antennas were filled with a dielectric material with a refractive index of 1.45. (d) AFM map of a gold nanostrip surface coated with SAM ODT. (e) Comparison of the experimental reflection spectrum of a sensor to the simulated reflection spectra with smooth or rough gold nanostrip surface. The nanostrip width of this sensor is 380 nm.

FIG. 59: Permittivity of gold by Olmon. The solid lines are the Drude model of gold while the data points are experimental data.

FIG. 60: (a) Reflection spectra of a thin proline film on a gold mirror at 80° incident angle, for both p- and s-polarizations. (b) Extracted relative permittivity of proline based on the Lorentz model.

FIG. 61: (a) Reflection spectrum of the bare device (red-shifted) and that of the same device with the proline precipitate. (b) Reflection spectrum of the bare device and its polynomial fit, as well as the difference between them. (c) Reflection spectra of the device with the proline precipitate before and after applying the correction for the water vapor absorption lines. (d) Reflection spectrum of the device with the proline precipitate after correction and the red-shifted polynomial fit of the bare device reflection spectrum.

FIG. 62: Simulations of the sensor structures by taking into account the surface roughness of gold nanostrips. (a) An example of the generated rough surface of the gold nanostrips used in our simulations. (b) A representative cross-sectional view of the profiles of the rough gold nanostrip surface, the coated SAM ODT, and the corresponding liquid gallium surface in our simulation model. (c) Simulated reflection spectra of nano-patch antennas based on several random rough surfaces. The blue dashed line is the measured reflection spectrum. (d) Simulated reflection spectra of nano-patch antennas with an effective air gap of varied thickness between the SAM ODT and the liquid gallium surface.

FIG. 63: A chart depicting a method according to an embodiment of the present disclosure.

### DETAILED DESCRIPTION OF THE DISCLOSURE

Raman spectroscopy can provide additional molecular information on the analyte as a complementary way to IR absorption spectroscopy. Similar to IR absorption spectroscopy, Raman spectroscopy can realize non-destructive, label-free identification of the molecular species and hence has been widely used in label-free sensing applications, especially in biological and life science. However, Raman scattering is a relatively weak process and it is still challenging to directly detect monolayers or even sub-monolayer of molecules such as proteins, DNA, or RNA. Since the Raman scattering process is approximately proportional to the fourth power of the electric field experienced by the analyte molecules, enhancement of the electric field in the metal nanostructure due to the excitation of the surface plasmons can effectively boost the Raman signal. The photonics resonators, which can support the surface plasmons, can effectively increase the electric field and hence increase the enhancement factor of the Raman signal. To obtain sensors with higher sensitivity, researchers have studied a variety of photonic structures to increase the enhancement of the electric field. One of the most effective ways to increase the electric field is to design photonic structures with smaller gaps as the hot spots. Under the framework of classical electromagnetic theory (i.e., ignoring non-local effect and quantum tunneling effect when the gap size g>0.5 nm), the electric field intensity increases as the gap decrease with the relation

$${\vert E \vert}^{2} ∝ \frac{1}{{g}^{p}}$$

with p=1.2˜1.5. Photonics structures with a nanometric size gap can significantly boost the electric field in the hot spot and enhance the nanoscale light-matter interactions, which enable ultrasensitive sensing applications including single-molecule detection. However, the difficulty of delivering the analyte into the nanometric gap increases with the decrease of the gap size, especially when the molecular size is comparable with the gap size. Because only the analyte in the hot spots of the resonator will experience the strong enhancement of the Raman signal, the inefficient delivery of the analyte to the nanometric gaps will cause lower signal enhancement and hence lower sensitivity. In addition, the fabrication and the mass production of nanostructures with nanometric gaps is still challenging.

In this disclosure, an enhancement strategy using liquid metal (e.g., liquid gallium) is presented for a SERS sensor. Thanks to its fluidic property and high conductivity, liquid gallium was used to sandwich the analyte adsorbed on the metal surface, and then nanometric gaps i.e., hot spots formed, which can confine highly enhanced electric field and hence boost the Raman scattering from the molecules in the gaps. The simulation results in Section 3.3 of the Additional Specification (below) show that the electric field of nanoparticles such as sphere or spherical dimers will increase after coating with the liquid gallium with the excitation of the laser beam at 785 nm. In our experiment, the SERS substrate was prepared with the cost-effective lithography-free rapid thermal annealing (RTA) method. The experimental results show that the Raman scattering can be further enhanced by roughly one order of magnitude by introducing the liquid gallium and the spatial uniformity of the Raman signal was also improved. Liquid gallium provides us with a new path to effectively enhance the electric field and achieve higher sensitivity for the SERS sensors. In addition, this cost-effective approach can also be applied to a variety of photonics systems such as nano-cavities for realizing strong light-matter coupling, nonlinear optics, quantum optics, etc.

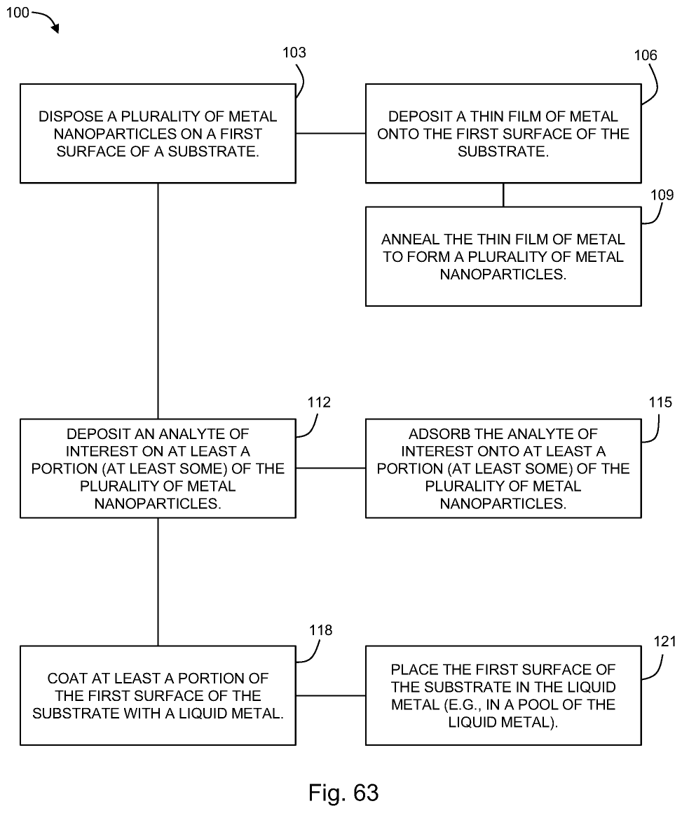

With reference to FIGS. 2 and 63, in a first aspect, the present disclosure may be embodied as a method 100 of preparing a sample for Raman spectroscopy. The method 100 includes disposing 103 a plurality of metal nanoparticles on a first surface of a substrate. The substrate may be selected to be suitable for Raman spectroscopy using light transmitted through the substrate. For example, the substrate may be made from glass, fused silica (e.g., quartz), etc. The metal nanoparticles may be gold or silver. Other suitable nanoparticle materials may be used, such as SERS-active materials. The metal nanoparticles may be disposed on the substrate by lithography, metal deposition, lift-off, or other techniques. In an example, the metal nanoparticles are disposed on the first surface of the substrate by depositing 106 a thin film of the metal onto the first surface of the substrate and then annealing 109 the thin film of metal to form nanoparticles of the metal. In an example, the metal thin film is deposited on the substrate by sputtering, and then rapid thermal annealing (RTA) is used to form nanoparticles (for example, at temperatures of 600° C. to 1000° C., for 30-60 seconds, however other temperatures (higher or lower) and times (longer or shorter) may be used).

The plurality of metal nanoparticles may be configured in various ways. For example, in some embodiments, the plurality of metal nanoparticles vary in diameter (non-uniform). In other embodiments, the plurality of metal nanoparticles are uniform in diameter (e.g., a diameter of each metal nanoparticle is within 3%, 5%, 7%, 8%, 9%, 10%, 15%, or 20% (in various embodiments) of the diameters of the other metal nanoparticles or of the average diameter). In various embodiments, the average diameter of the plurality of metal nanoparticles is between 20 nm and 30 nm, inclusive, or 20 nm and 50 nm, inclusive, or 10 nm and 50 nm, or 30 nm and 100 nm, inclusive, or 10 nm and 150 nm, inclusive or any ranges or sizes therebetween. In various embodiments, each nanoparticle of the plurality of metal nanoparticles may have a diameter in the range of 10 nm to 100 nm, inclusive, or 20 nm and 80 nm, inclusive, or 50 nm and 150 nm, inclusive, or 30 nm and 80 nm, or 30 nm and 100 nm, inclusive, or any ranges or sizes therebetween (or diameters greater than or less than these diameters). In various embodiments, each nanoparticle of the plurality of metal nanoparticles may have a diameter of less than 10 nm, less than 20 nm, less than 50 nm, or less than 100 nm. In various embodiments, the metal nanoparticles of the plurality of metal nanoparticles are separated from one another by distances ranging from 2 nm to 100 nm, inclusive, or 10 nm to 100 nm, inclusive, or 20 nm to 80 nm, inclusive, or 20 nm to 150 nm inclusive, or any ranges or distances therebetween (or distances greater than or less than these distances).

An analyte of interest is deposited 112 on the plurality of metal nanoparticles. For example, a solution comprising the analyte of interest may be contacted with the plurality of metal nanoparticles. The analyte of interest may be adsorbed 115 onto the plurality of metal nanoparticles. It should be noted that the analyte of interest may comprise a plurality of analyte molecules.

The method 100 includes coating 118 at least a portion of the first surface of the substrate with a liquid metal. In this way, the plurality of metal nanoparticles and the analyte of interest are encapsulated between the first surface of the substrate and the liquid metal. The liquid metal may be, for example, liquid gallium, liquid gallium-indium (liquid gallium-indium eutectic), or other materials suitable to form a liquid ground plane for use with a Raman system. In some embodiments, the first surface is coated 118 by placing 121 the first surface of the substrate in the liquid metal (i.e., a pool of the liquid metal).

In another aspect, the present disclosure may be embodied as a sensor for Raman spectroscopy made according to any of the methods disclosed herein.

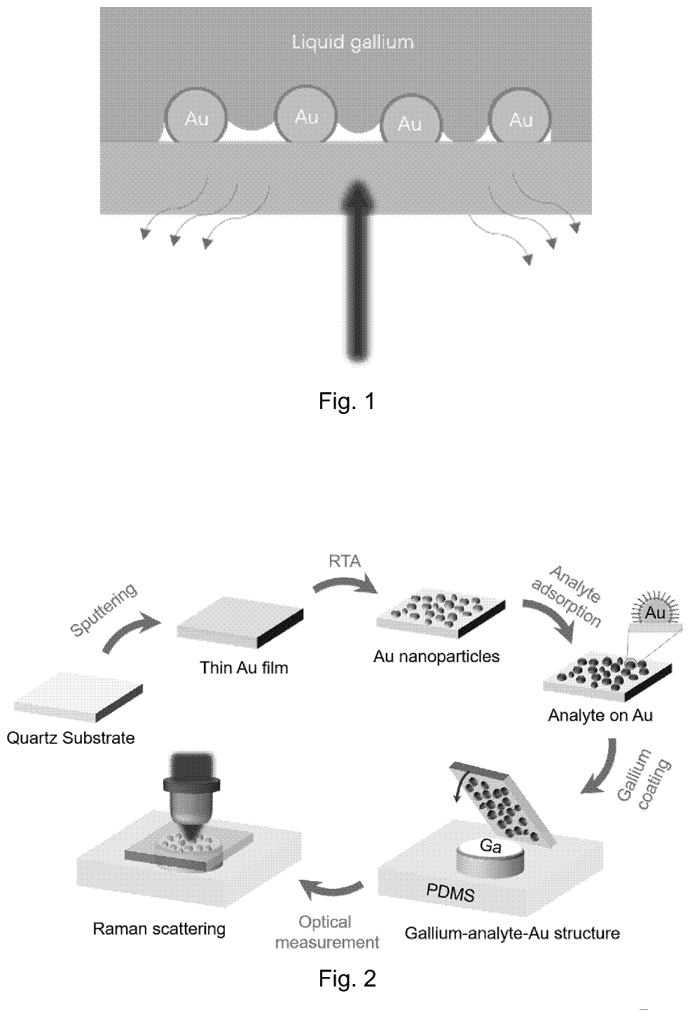

In another aspect, the present disclosure may be embodied as a sensor for Raman spectroscopy (see, e.g., FIG. 1). The sensor has a substrate; a plurality of metal nanoparticles disposed on at least a portion of a first surface of the substrate; an analyte of interest adsorbed on the plurality of metal nanoparticles; and a liquid metal coating on at least a portion of the first surface of the substrate thereby encapsulating the plurality of metal nanoparticles and analyte of interest between the substrate and the liquid metal. The metal nanoparticles may be of any of the configurations described herein (e.g., size, spacing, material, etc.)

In another aspect, the present disclosure may be embodied as a method of preparing a sample for Raman spectroscopy. The method includes disposing a plurality of SERS-active nanoparticles on a first surface of a substrate. The substrate may be selected to be suitable for Raman spectroscopy using light transmitted through the substrate. For example, the substrate may be made from glass, fused silica (e.g., quartz), etc. The SERS-active nanoparticles may be gold, silver, or any other SERS-active material. The SERS-active nanoparticles may be disposed on the substrate by lithography, metal deposition, lift-off, or other techniques. In an example, the SERS-active nanoparticles are disposed on the first surface of the substrate by depositing a thin film of a SERS-active material onto the first surface of the substrate and then annealing the thin film to form nanoparticles of the SERS-active material. In an example, the thin film is deposited on the substrate by sputtering, and then rapid thermal annealing (RTA) is used to form nanoparticles (for example, at temperatures of 600° C. to 1000° C., for 30-60 seconds, however other temperatures (higher or lower) and times (longer or shorter) may be used).

The plurality of SERS-active nanoparticles may be configured in various ways. For example, in some embodiments, the plurality of SERS-active nanoparticles vary in diameter (non-uniform). In other embodiments, the plurality of SERS-active nanoparticles are uniform in diameter (e.g., a diameter of each SERS-active nanoparticle is within 3%, 5%, 7%, 8%, 9%, 10%, 15%, or 20% (in various embodiments) of the diameters of the other SERS-active nanoparticles or of the average diameter). In various embodiments, the average diameter of the plurality of SERS-active nanoparticles is between 20 nm and 30 nm, inclusive, or 20 nm and 50 nm, inclusive, or 10 nm and 50 nm, or 30 nm and 100 nm, inclusive, or 10 nm and 150 nm, inclusive or any ranges or sizes therebetween. In various embodiments, each nanoparticle of the plurality of SERS-active nanoparticles may have a diameter in the range of 10 nm to 100 nm, inclusive, or 20 nm and 80 nm, inclusive, or 50 nm and 150 nm, inclusive, or 30 nm and 80 nm, or 30 nm and 100 nm, inclusive, or any ranges or sizes therebetween (or diameters greater than or less than these diameters). In various embodiments, each nanoparticle of the plurality of SERS-active nanoparticles may have a diameter of less than 10 nm, less than 20 nm, less than 50 nm, or less than 100 nm. In various embodiments, the SERS-active nanoparticles of the plurality of SERS-active nanoparticles are separated from one another by distances ranging from 2 nm to 100 nm, inclusive, or 10 nm to 100 nm, inclusive, or 20 nm to 80 nm, inclusive, or 20 nm to 150 nm inclusive, or any ranges or distances therebetween (or distances greater than or less than these distances).

An analyte of interest is deposited on the plurality of SERS-active nanoparticles. For example, a solution comprising the analyte of interest may be contacted with the plurality of SERS-active nanoparticles. The analyte of interest may be adsorbed onto the plurality of SERS-active nanoparticles. It should be noted that the analyte of interest may comprise a plurality of analyte molecules.

The method includes conformally coating at least a portion of the first surface of the substrate with a conductor. In this way, the plurality of SERS-active nanoparticles and the analyte of interest are encapsulated between the first surface of the substrate and the conductor. The conductor may be a liquid conductor, such as a liquid metal conductor (e.g., liquid gallium, liquid gallium-indium (liquid gallium-indium eutectic), etc.) In some embodiments, the liquid conductor solidifies after coating. In some embodiments, the conductor is a solid conductor. In some embodiments, the first surface is coated by placing the first surface of the substrate in a liquid conductor (i.e., a pool of the liquid conductor).

In another aspect, the present disclosure may be embodied as a system for surface enhanced Raman spectroscopy (SERS), any of the SERS sensors described herein or a SERS sensor made using any of the methods described herein.

### Device Design and Fabrication

### Device Design Rationale

FIG. 1 shows a schematic of the liquid gallium based SERS sensor structure. The proposed sensor chip is based on commonly used metal nanoparticles on a glass substrate. The analyte film was initially coated on gold nanoparticles with methods such as spin-coating or adsorption. After covering the sensor chip with liquid gallium, the analyte film was sandwiched between the liquid gallium and gold nanoparticles, and hence the nanometric gaps formed with the analyte film filled in. The introduction of liquid gallium into the sensor design has a few advantages. Because of the fluidity properties of the liquid gallium, the liquid gallium can tightly contact the analyte film and form a nanometric gap the same size as the molecular size. Under laser excitation, these nanometric gaps can confine most of the electric field and enhance the intensity. Compared with other kinds of resonators such as dimer resonators or SSRs which only use a small portion of the area as the hot spot, the overlap between the electric field and the analyte film is relatively high and hence can make efficient use of the available analyte. Also, liquid gallium is a cost-effective material compared with the noble metals and is environmentally friendly and biologically compatible. Finally, the liquid gallium can easily be removed from the analyte surface, which makes both gallium and device chips re-usable.

### Device Fabrication

Non-limiting, example device chips were fabricated with a lithography-free method. A thin gold film was deposited on a fused silica substrate. Then rapid thermal annealing was employed to transform the gold thin film to gold nanoparticles on the substrate. The analyte film was coated on the surface of gold nanoparticles with methods such as adsorption or spin coating. Then the device chip was placed on the liquid gallium in a Polydimethylsiloxane (PDMS) well after removing the oxide layer from the liquid gallium surface with a plastic scraper. After forming good contact with the gallium, the device chips were put under the Raman microscope for measuring the Raman spectrum.

Substrate cleaning: As the first step of the device fabrication, the substrate was cleaned in an acetone sonication bath for around 10 minutes and then rinsed in isopropyl alcohol (IPA) followed by blowing dry with nitrogen. Then, the oxygen plasma cleaning of the substrate surface was conducted to further remove surface organic residue and improve the surface potential.

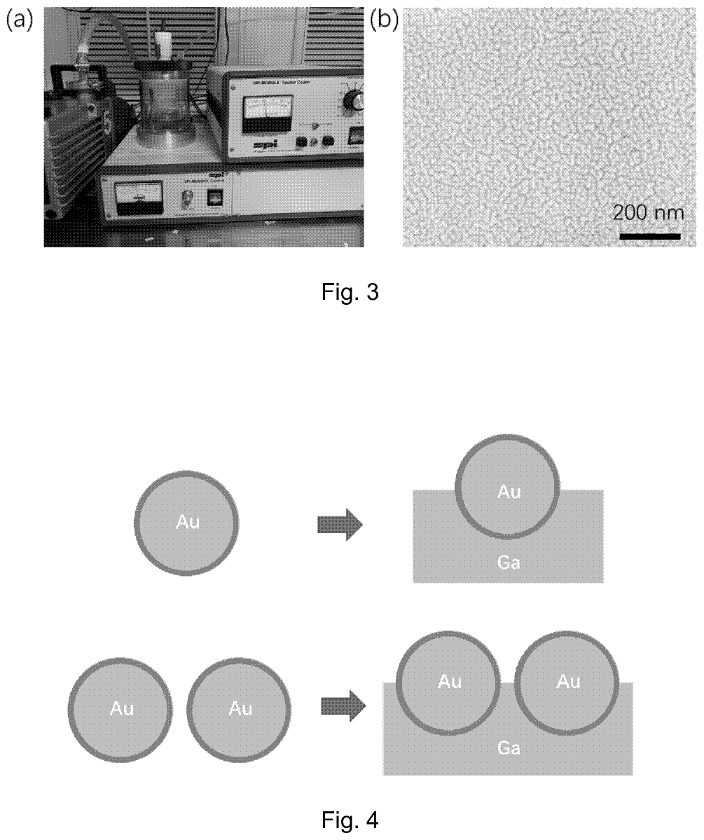

Metal deposition: SPI-MODULE sputter coater was used to deposit the gold thin film on the substrate (see FIG. 3(a)). This simple cost-effective sputtering system can produce a thin gold film of good quality. After cleaning, the substrates were put into the sputtering system and then were deposited at a constant current of 20 mA for varied times. FIG. 3(b) shows a SEM image of the gold thin film after depositing for 90 seconds. The homogenous gold thin film and the grain boundary can be observed in the SEM image.

Rapid thermal annealing: The chips with the deposited gold thin film were put into a rapid thermal annealing system. The density and the geometry of the formed nanoparticles can be affected by the annealing recipe as well as the thickness of the gold film. Since the melting point of the gold in the nanoscale depends on the thickness of the film, it is already enough to break the uniform thin film and form the gold nanoparticles even if the annealing temperature is lower than the gold melting point. To anneal the samples, the temperature increases to the target temperature (range of 600° C. to 1000° C.) within 30 seconds and then holds constant for another 30 seconds before cooling down to room temperature naturally.

Coating of the analyte: To demonstrate the sensor performance, biphenyl-4-thiol (BPT) molecules were used to test the sensor performance. BPT is an alkyl-thiol that forms a self-assembled monolayer (SAM) with an approximate thickness of 1 nm on the gold surface by forming strong S—Au bonds. A BPT solution was prepared by dissolving 56 mg solid BPT into 300 mL methanol solvent to form the 1 mM solution. Before coating BPT molecules, the sensor chips were cleaned in acetone and IPA to remove organic residuals, followed by oxygen plasma cleaning to further remove organic residuals and modify the surface potential. The sensor chips were immediately immersed in the 1 mM BPT solution for at least 4 hours. After being taken out of the BPT solution, the sensor chips were rinsed with pure methanol thoroughly to remove unbound molecules and then were blown dry with the nitrogen gun.

Liquid gallium contact formation: The contact between liquid gallium and the sensor chips was formed just before the optical measurement. The liquid gallium was introduced into a PDMS well and the surface oxide layer was removed by sweeping a plastic rod on the liquid gallium surface. Then the sensor chip was placed facing down on the liquid gallium. Since the gold nanoparticle surface was coated with the uniform BPT SAM film, which separated the gallium from the gold nanoparticles and formed uniform gaps between gold and gallium. After optical measurement, the liquid gallium could be completely removed from the device chip since the gallium does not wet with a variety of organic materials including the BPT layer.

### Numerical Simulations

The numerical simulations with the FDTD method were conducted to investigate further field enhancement by introducing the liquid gallium on the gold nanoparticles. Two typical structures of the randomly distributed gold nanoparticles were simulated: individual nanosphere, and sphere dimer with a separation gap of 10 nm. In the simulation, a 2 nm thick dielectric layer with relative permittivity of 2 was coated on the surface of gold nanosphere, which represents the thin analyte film adsorbed on the gold particles. For the simulation of nanostructures with a liquid gallium ground plane, the liquid gallium was placed to cover the lower half of the nanospheres (see FIG. 4). Although the gallium profile and the nanoparticle distribution in reality may be much more complex, these simulations can reflect, to some degree, the enhancement of the electric field by introducing the liquid gallium. The permittivity of gold was derived from previous work while the gallium is modeled using the Drude model with a plasma frequency at 14.5 eV and a damping rate at 0.968 eV. The boundary condition in the x and y directions were set to be periodic conditions while in the z direction the PML boundary condition was used. The mesh size near the metal sphere was set to be 0.3 nm. The near field monitor was put in the center of the gold sphere along the electric field direction of the excitation wave and the far field monitor was put 1 μm away from the light source to collect the reflected wave.

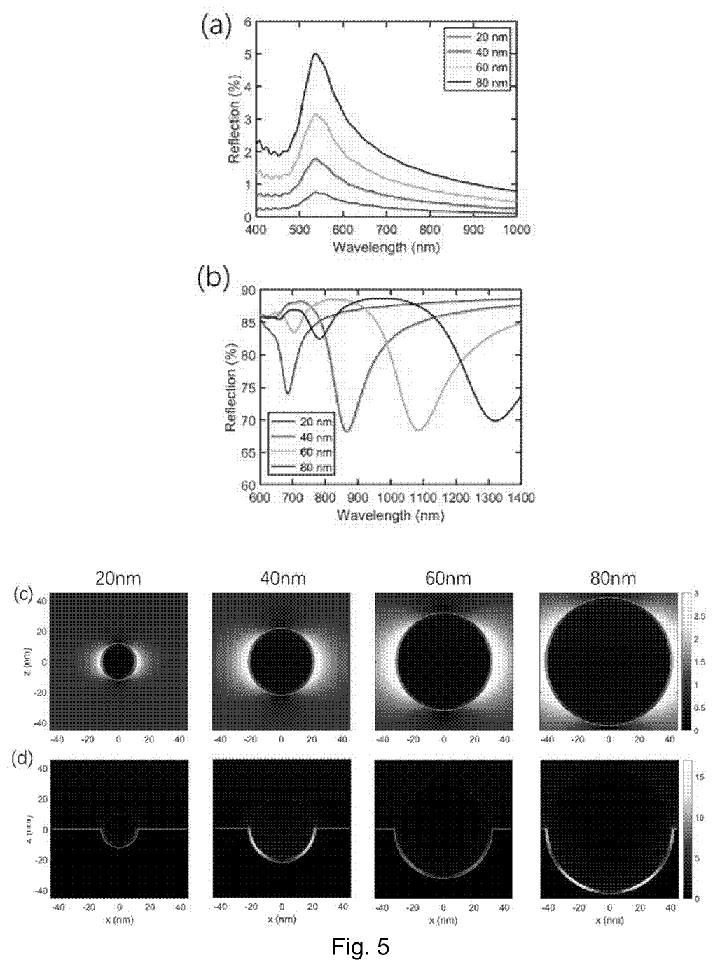

FIGS. 5(a) and (b) give the simulated reflection spectra of the nanosphere before and after coating with liquid gallium, with the diameter of the nanosphere ranging from 20 nm to 80 nm before and after coating with liquid gallium. For the nanosphere only, the reflection peaks are fixed around the wavelength of 540 nm with the change of particle size. The intensity of the reflection peaks is increased due to the increase of the cross-section with the increase in the diameter of the nanosphere. When the nanosphere is covered with gallium, the reflection dips shift significantly from around 700 nm to 1300 nm with the increase of the sphere diameter, and the intensity of resonance also becomes stronger. FIGS. 5(c) and (d) show the simulated electric field distribution of the nanosphere with a diameter ranging from 20 nm to 80 nm at a wavelength of 785 nm, which is the excitation wavelength of the laser in Raman spectroscopy. The excited electric fields of nanospheres with different diameters were located on the left and right side of the sphere and the enhancements of electric fields inside of the analyte film are almost at a similar level around 1.5. As a comparison, after coating with liquid gallium, almost all the electric fields are confined in the 2 nm gap between the gallium and gold sphere and the maximum enhancement of the electric field for the nanosphere with different diameter will reach around 18 are in the range of 10 to 18, which is over one order of magnitude stronger than that of nanosphere only. Considering the fourth power relation between the electric field and Raman scattering, the Raman signal of the nanosphere will be further enhanced over almost 104 times by introducing the liquid gallium.

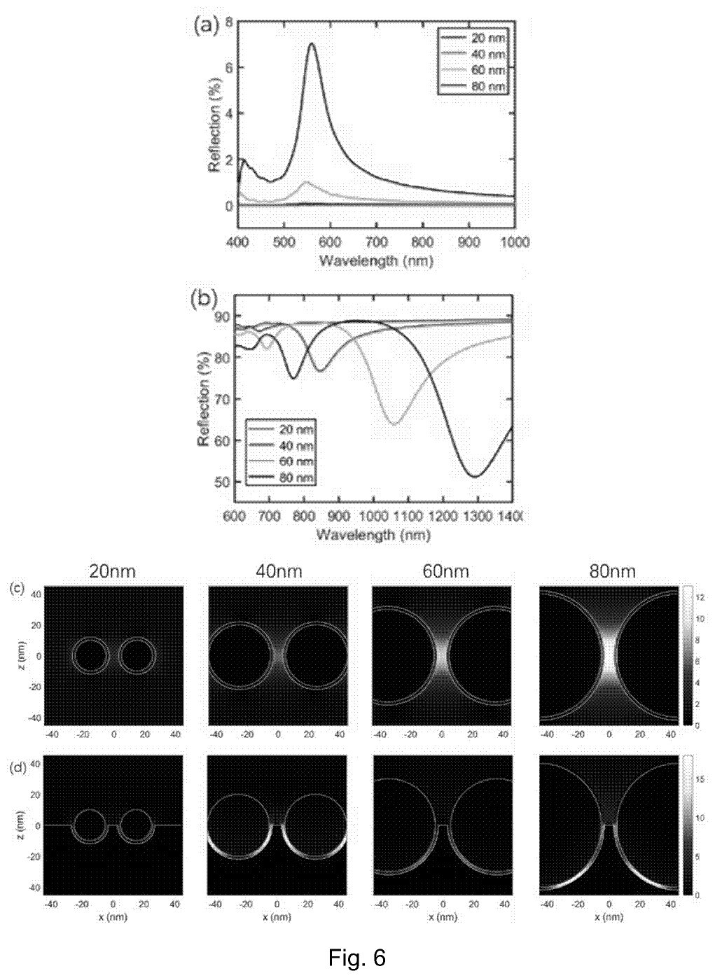

Simulated reflection spectra of sphere dimers are shown in FIG. 6(a). Compared with the nanosphere only, the dimer structures have the same resonant wavelength at around 540 nm but the reflection peaks of the dimer have higher intensity and quality factors, which indicate the stronger resonance in the dimer. The electric field of the dimer is mainly confined near the gap region and the field enhancements become larger with the increase of the sphere diameter (FIG. 6(c)). Although the maximum electric field enhancement in the gap is predicted to be close to 13 with the sphere diameter of 80 nm, the electric field, the maximum electric field enhancement in the analyte film is only 6 times. As a comparison, the dimers after coating with liquid gallium have stronger resonant dips (around 50%) and the frequency of resonant dips will decrease with the increase of the sphere diameter FIG. 6(b). After coating with liquid gallium and forming the nanometric gap, the electric field will be mainly confined in the gap between gallium and gold sphere. Since the diameter size of the sphere dimer changes the resonant frequency, electric field enhancement at 785 nm in the nano-gap also varied from around 10 times to 20 times with the change of diameter size. Hence the overall electric field in the analyte film will lead to an additional 2˜ 3 times enhancement by coating with the liquid gallium, which will contribute to a few tens of times enhancement of the Raman signal.

### Experiment Results

### Measurement Configuration of Raman Spectroscopy

Raman spectra were measured using a confocal Renishaw in Via Raman microscope equipped with a 785 nm laser. A 20× objective lens with a numerical aperture 0.4 was used to focus the lasers beam on the sample and also collect the scattering signals from the sample surface. A 1200 lines per millimeter grating was used to disperse the Raman signal. To have a direct comparison of the Raman signal, we measured the spectra at the same position of the sensor chip before and after coating with liquid gallium. In the experiment, the power was set to be 15 mW with the integration time of 1 s. 2D Raman mapping was performed over a 100 μm×100 μm area with a step size of 10 μm for each measurement.

### SERS Spectrum

Since the density, size and shape of the nanoparticles on the substrate play a significant role for the enhancement of the SERS, the sensor chips with varied morphology of nanoparticle was prepared for the Raman spectroscopy measurement.

### Sensor Chip N1

The sensor chip N1 was prepared with around 60 seconds of sputtering of the gold, followed by the RTA at 600° C. The inset of FIG. 5(a) gives the SEM image of the sensor chip after annealing. Gold nanoparticles of varied sizes can be observed on the substrate. The distribution map of particle size on the sensor chip is estimated and shown in FIG. 5(a). There are 226 nanoparticles in the area of 0.45 μm² with an average particle size of around 26 nm.

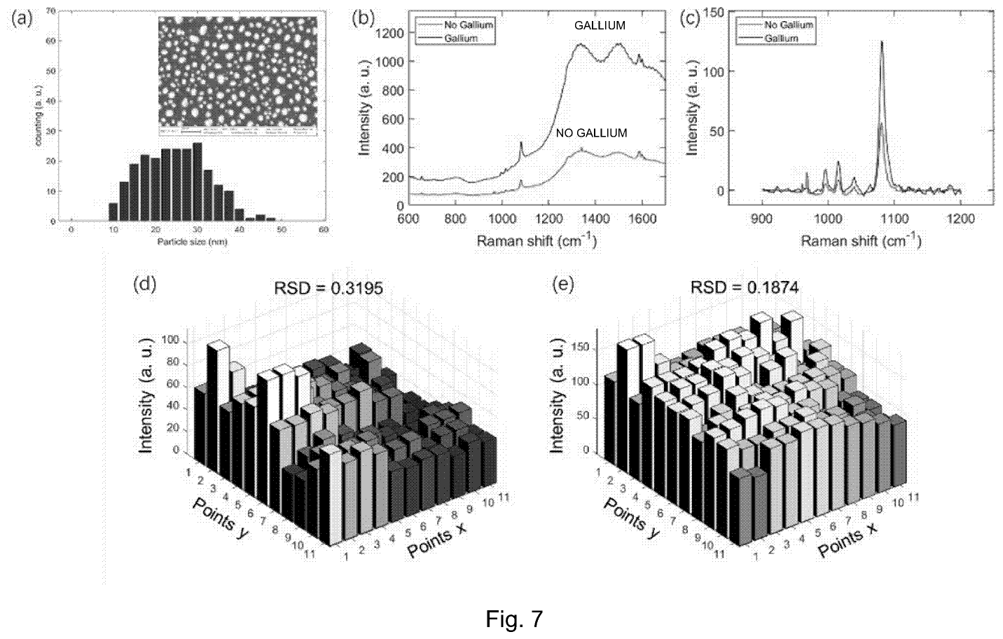

After coating with SAM of BPT, sensor chip N1 was characterized with Raman spectroscopy. Measured Raman spectrum of SAM BPT coated on gold nanoparticles is shown as the lower curve in FIG. 7(b). The characteristic Raman bands at around 1080 cm⁻¹ and 1587 cm⁻¹ can be easily observed, which correspond to the breathing mode and stretching motion of the phenyl ring in the BPT molecule, respectively. In addition to the Raman scattering peaks, the spectra contain a broadband background signal which mainly comes from the fluorescence and Raman scattering of the substrate and the gold nanoparticles. As a comparison, the sensor after coating with gallium shows higher Raman scattering signal as well as the fluorescent light intensity (see the upper curve in FIG. 7(b)). To directly compare the Raman signal only for the sensor chip before and after coating liquid gallium, the Raman peaks were extracted with the polynomial fitting method to remove the background signal (see FIG. 7(c)). The main Raman peak at 1080 cm⁻¹ and the other three weak Raman peaks (996 cm⁻¹, 1015 cm⁻¹, and 1039 cm⁻¹) were roughly enhanced around 2.3 times by introducing the liquid gallium. Spatial uniformity of Raman signal over the sensor chip can be another important aspect for the SERS substrate. A sensor chip with high spatial uniformity of Raman signal can perform the sensing application with quantitative analysis. Since the gold nanoparticles prepared by the RTA method are inherently randomly distributed, the localized field over the laser beam focus area highly depends on the position of the sensor chip, which may make it challenging to perform the quantitative sensing. By introducing the liquid gallium on the sensor chip, the spatial uniformity of the Raman signal can be improved. A 2D mapping of Raman signal at 1080 cm⁻¹ for sensor before and after coating gallium was plotted in FIGS. 7(c) and (e). The relative standard deviations of Raman intensities of sensor chip before and after coating gallium are 31.9% and 18.7%, respectively. This indicates that coating of gallium can effectively improve the spatial uniformity of the Raman signal.

### Sensor Chip N2

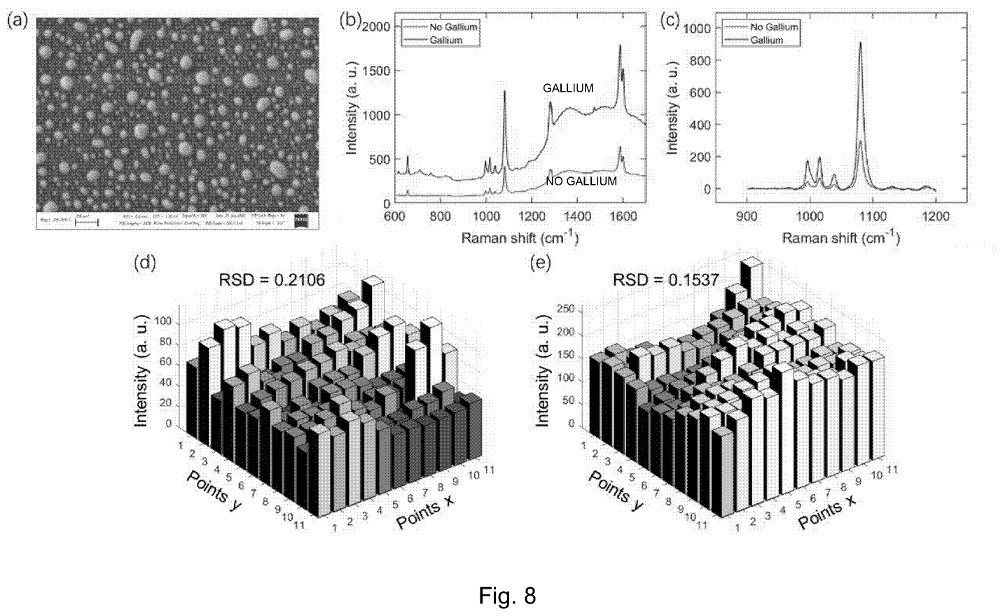

The insertion of small nanoparticles into the large nanoparticles can provide higher Raman signal enhancement due to the decrease of the gap between metallic nanoparticles. A sensor chip N2 was prepared with a two-step process of gold deposition. Relatively large gold nanoparticles were formed by a 90 s sputtering of gold and followed by RTA at 600° C. Then, the sensor chip was sputtered with 30 s gold and annealed at 600° C. again to create smaller nanoparticles among the larger nanoparticles. FIG. 8(a) shows an SEM image of sensor chip N2. A high density of small nanoparticles with a diameter of below 10 nm were immobilized amongst the larger nanoparticles. Because the distance between the small nanoparticles is small, the electric field in such a small gap will be highly confined and hence provide higher Raman signal enhancement. The measured Raman spectra of the sensor chip coated with BPT molecules are shown in FIG. 8(b). After coating with liquid gallium, the sensor chip shows a stronger background signal as well as enhanced Raman signals. Compared with sensor chip N1, sensor chip N2 had a similar background signal level while the Raman peaks are much stronger, which is around 2˜3 times stronger than that of sensor chip N1. Also, additional Raman peaks (i.e., 656 cm⁻¹, 1281 cm⁻¹, and 1471 cm⁻¹) can be observed from sensor chip N2 resulting from the stronger Raman signal. The extracted Raman peaks signal around 1080 cm⁻¹ are given in FIG. 8(c). All four vibrational peaks were enhanced after coating with gallium and the enhancement factors were around 3.3×. 2D mapping of Raman signal at 1080 cm⁻¹ of sensor chip N2 also shows the improvement in the spatial uniformity of Raman signal by introducing the gallium.

### Sensor Chips N3-N6

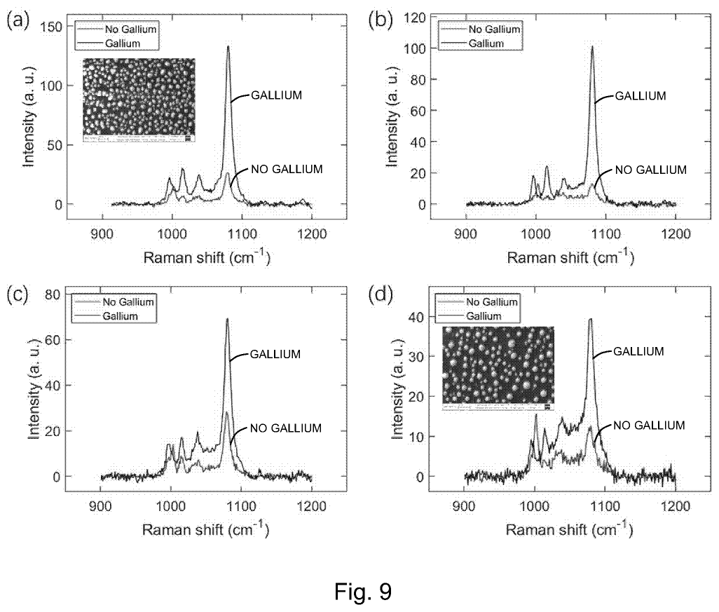

Because a glass substrate would melt under annealing temperatures, fused silica substrates were used in the following experiments. Four sensor chips were sputtered with gold for around 90 seconds and then annealed at temperatures of 700° C. (chip N3), 800° C. (chip N4), 900° C. (chip N5), and 1000° C. (chip N6). The SEM images of the sensor chips after annealing are shown in the inset of FIG. 9. Compared with the sensor chips made using lower annealing temperatures, sensor chip N6 had a lower density of nanoparticles but a larger particle size. After coating with BPT, the Raman spectra of all the device sensor chips were measured with Raman spectroscopy and the extracted Raman peaks are shown in FIGS. 9(a)-9(d). Before coating with liquid gallium, the Raman signal was relatively weak and all of the sensor chips had an intensity of only ˜20 at the Raman peaks of 1080 cm⁻¹. Compared with the glass substrate, a new peak around 1000 cm⁻¹ appeared in the measured spectrum, which is likely a result of defects of the substrate. After coating with liquid gallium, the sensor chips show a stronger Raman signal and the sensor chips with a lower annealing temperature had a higher Raman signal at the peak of 1080 cm⁻¹. The calculated enhancement factors of the Raman signal by introducing gallium for sensor chips from N3 to N6 were 5.1, 7.4, 2.5, and 3.1, respectively.

### Sensor Chip N7

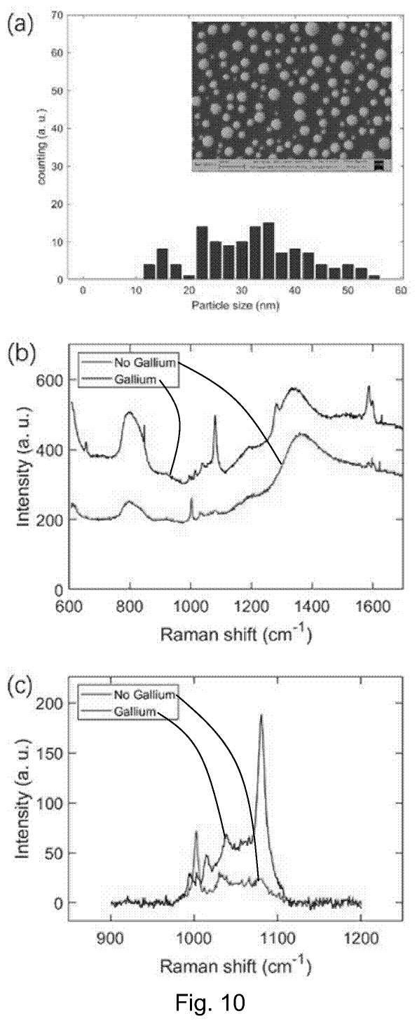

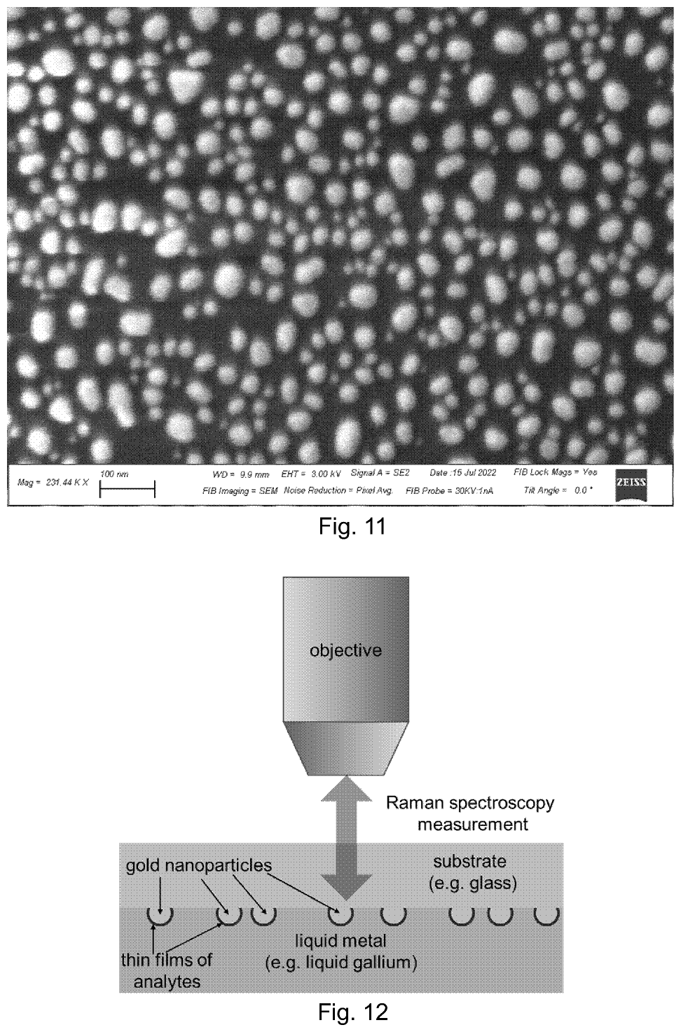

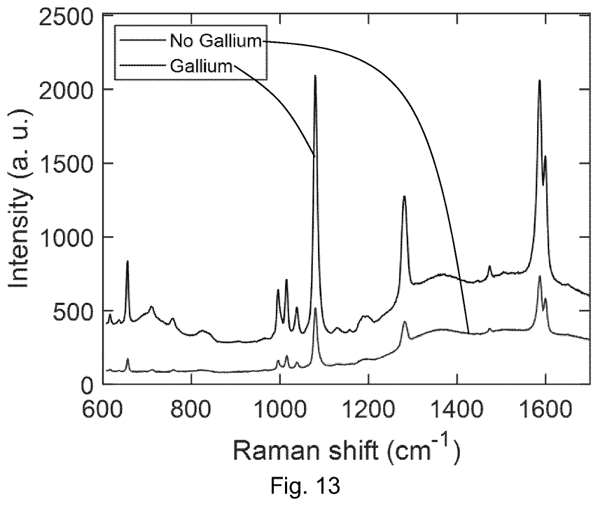

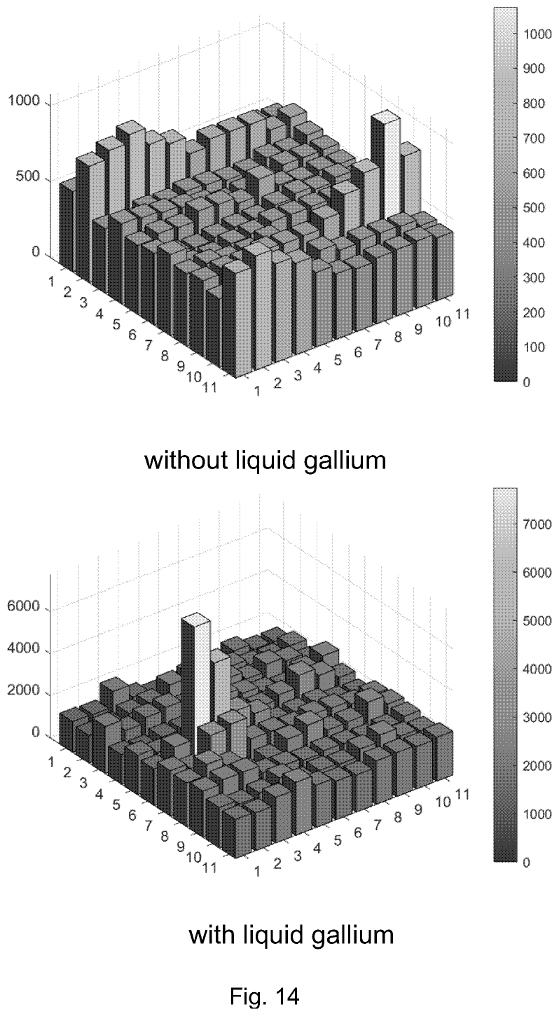

To investigate the sensing performance of a sensor with a larger particle size, a sensor chip (N7) was prepared using a thicker gold film (deposited by sputtering for 120 seconds). After annealing at a temperature of 1000° C., the sensor chip was characterized with SEM and the surface image is shown in the inset of FIG. 10(a). Particle size distribution was statistically estimated according to the SEM image and is shown in FIG. 9(a). The average particle size was large (around 33 nm) and also the density of the nanoparticles was low compared with the previous sensor chips. In the measured Raman spectrum (see FIG. 9(b)), only the broadband background signal and substrate defect Raman peak at 1000 cm⁻¹ can be observed due to the low SERS enhancement factor. This is consistent with the simulation results that the spherical nanoparticles have much lower field enhancement compared with dimer particles. Since the sensor chip had a poor limit of the detection, the vibrational Raman peaks were relatively weak and hard to be observed. After coating with liquid gallium, both the broadband fluorescence signal and the Raman signal were enhanced such that the Raman peaks easily observed. After extracting the Raman peaks from the measured spectra (see FIG. 10(c)), it is apparent that the Raman peak at 1080 cm⁻¹ was enhanced around 8 times after coating with liquid gallium.

### SUMMARY

We experimentally demonstrated a high-performance SERS substrate with an enhancement method using liquid gallium. Based on the cost-effective RTA annealing produced gold nanoparticle substrate, the proposed sensor structures are targeted to provide high Raman signal enhancement for the analyte thin film coated on the metal nanoparticle. Thanks to its fluidic nature, the liquid gallium can conformally cover the analyte film on the gold nanoparticles, leading to the formation of nanometric gaps, which support highly enhanced electric field and hence boost the Raman signal of the analyte in the gaps. According to the simulation, the formation of the nanometric gap after covering liquid gallium can significantly boost the electric field enhancement and hence increase the Raman scattering process by an estimated 4 orders of magnitude for the single sphere and 2 orders of magnitude for the dimer. The measured Raman spectra from the gold nanoparticles coated with the SAM BPT show that the introduction of the liquid gallium can increase the Raman signal by one order of magnitude and improve spatial uniformity. Also, our liquid gallium based SERS substrate is cost-effective considering that the price of liquid gallium is low compared with noble metals and the sensor chips fabricated by the thin film sputtering and RTA method can be mass produced with the low cost. The demonstrated cost-effective and reproducible method of employing liquid metals to boost field confinement and enhancement in the nanophotonic structure provides a new platform for various SERS sensing applications.

### 1. Introduction

### 1.1 Development of the Vibrational Spectroscopy

Raman spectroscopy and infrared (IR) absorption spectroscopy are powerful tools to provide the optical spectra of analyte that contains molecular or lattice vibrational information. Hence, they are widely used in research institutions as well as industry for label-free, nondestructive, and unambiguous identification of molecular species. Both Raman spectra and IR absorption spectra are the results of the vibrational movement of molecules. IR absorptions are caused by induced change of dipole transition moments related to the molecular vibrations under the excitation of the infrared wave. Raman scattering occurs when the sample is illuminated with light and the inelastic photon scatterings occur due to the excitation of vibrational modes in the molecules.

The early use of the IR spectroscopy started with the experiments of Sir Willian de Wiveleslie Abney in 1881 and William W. Coblentz in 1903, which recorded the IR spectra of inorganic and organic compounds substances and indicated the absorption bands from different types of bonds. Then the demand for rapid analysis in the industry such as synthetic rubber stimulated the commercial development of IR spectroscopy, which further expanded the acquisition of IR spectra and motivated deeper theoretical studies on IR spectra and their applications. Most of the IR instruments before the 1960s were based on the dispersive prism or grating monochromators. These dispersive instruments had a few drawbacks, such as limited operational frequency region, low resolution, and low signal-to-noise ratio. The introduction of Fourier-transform infrared (FTIR) spectroscopy is a breakthrough in the development of IR spectroscopy. The IR spectrum was obtained from the Fourier-transform of the interferogram collected by the interferometer of the FTIR system. However, the early calculation or the conversion from the interferogram to the frequency spectrum was slow and expensive because of the computation complexity of the Fourier transform. Then the invention of the fast Fourier transform (FFT) algorithm by James Cooley and John Tukey in 1965 and the rapid development of the computer decrease the calculation time and expense for the spectrum transform. Digilab company pioneered the world's first commercial FTIR spectrometer (Model FTS-14) in 1969. Now FTIR spectroscopy has been widely used in a variety of branches of science and technology.

The discovery of the Raman scattering or the Raman effect was later than that of IR absorption due to its weak process. This effect was predicted in 1925 1923 by Smekal and then was experimentally confirmed by Sir Chandrasekhara Venkata Raman in 1928. Since this effect usually happens in the visible light range and the experimental setup could be much more easily established than that in the IR region, this effect immediately aroused broad interest and was used as an additional tool in addition to IR spectroscopy. However, since the Raman scattering is a relatively weak process, Raman spectroscopy still need the professional users to operate due to the complex alignments and measuring procedures. As a result, Raman spectroscopy is only used in restricted research areas for a long period. After that, a few remarkable signs of progress make it no longer limited to a few specialized research laboratories. In 19426, Rank and Wiegand introduced the first photoelectric Raman spectrograph to improve the photometric accuracy in contrast to the earlier use of photographic plates as detectors. In the 1960s, the high-intensity light source which can significantly improve the Raman scattering intensity, are available due to the invention of the laser. In the 1980s, the use of CCD (charge-coupled device) detectors and holographic filters further increased the system accuracy and decreased the cost of the instruments. Now, this characterization technique has become increasingly more common in fundamental research as well as in industry.

Usually, the optical responses of molecular vibrations are relatively weak due to the low molecular absorption or scattering cross-sections, which means a considerably large amount of material is needed to achieve a reliable spectrum of the material. This is a fundamental limitation for vibrational spectroscopy and hence, for a long time, Raman and IR spectroscopy were only applied for characterizing structures of bulk solid or liquid samples. A remarkable progress for the vibrational spectroscopy is the introduction of the surface enhanced techniques. In 1974, Fleischmann et al. found unexpectedly enhanced Raman signal from molecules on the electrochemically roughened silver electrodes and in 1980 Hartstein et al. observed enhanced absorption with the surface enhanced infrared absorption spectroscopy (SEIRA) on the thin metal overlayers. These enhancements attributed the IR absorption enhancement and the Raman scattering enhancement to an electric field enhancement due to the excitation of surface plasmons on the island metal films. Then the research on surface enhanced Raman spectroscopy (SERS) and SEIRA spectroscopy are widely conducted. The rapid developments in nanoscience and nanofabrication technologies enable the preparation and fabrication of well-defined photonics nanostructures which typically contribute to much higher electric field enhancement larger than that of the rough metal thin-film structures. The optimization of the photonics nanostructures through the size, shape, morphology, and nanometersized gaps (nanogaps) in/between nanostructures, can lead to a higher electric field confined in the small portion area (i.e., hot spots) and hence significantly enhance the vibrational signals in the IR absorption and Raman spectra. In addition to the shape and the size of the nanostructures, the materials can also play a significant role in signal enhancement. Besides the commonly used noble metals such as Au or Ag, the emerging new material such as graphene, liquid gallium, and semiconductors also support the existence of surface plasmons and provide a new avenue for the sensing application.

### 1.2 Motivation and Outline of this Disclosure

The wide applications of vibrational spectroscopy promote the development of a variety of research fields. On the other hand, the development of these research fields creates new demands and requirements for vibrational spectroscopy with higher sensitivity. Taking the early diagnosis of lung cancer as an example, the concentration of protein biomarkers related to lung cancer can be as low as 1 ng/mL. It was still challenging to directly detect such low concentrated analyte solution as well as a monolayer or even sub-monolayer of molecules such as proteins, lipids, and DNA for applications in life science.

To obtain higher sensor sensitivity, the most directive way is to design the sensor structure with higher electric field enhancement. This can be typically achieved by designing the ultra-small gaps as the hot spot in viewing that electric field in the gaps depends on the gap size g following the relation |E_{loc}/E₀|²∝1/g^{p} with p≈1.2˜1.5. A variety of sensor designs with nanometric gaps have been proposed to improve sensor sensitivity. However, it will become much more challenging to deliver the analyte into the smaller hot spot of the sensor structure. The sensor devices can only “see” the existence of molecules located in the hot spots of the sensor device. The analyte molecules out of the hot spot region will not experience the strong electric field and hence will not contribute to the enhanced vibrational signal in the measured spectrum. This trade-off between the gap size and the analyte delivery efficiency limits the further improvement of the sensor structures.

The decrease in the gap size in sensor design can also lead to the challenge of the fabrication of sensor devices. The commonly used fabrication method for the nanostructure with a nanometric gap is the nanolithography method such as the electron beam lithography and focused ion beam etching, which are usually expensive for mass products. Hence, the sensor design with cost-effective fabricating methods to achieve nanogaps is still desired for the wide sensing application such as point-of-care sensing.

In addition to decreasing gap size in the sensor design, guiding more analyte molecules into the hot spot of the sensor device can also help to improve the sensing performance. When detecting the analyte molecules dispersed in the solution, only a small portion of the molecules distributed near the surface of the sensor device hot-spots lead to the vibrational signal. With the decrease in the concentration of the analyte solution, the total number of molecules near the hot spot decreases as well. To increase the sensor sensitivity, the researchers have demonstrated the sensor design by introducing active trapping mechanisms such as dielectrophoresis, optical trapping, and micro-bubble trapping methods. Although these technologies can effectively concentrate and deliver nanoobjects and large biomolecules (e.g., proteins and DNA), they still require external energy sources such as a laser or an applied voltage and are not suitable for trapping relatively small molecules. In addition, manipulating the droplet with help of the surface tension is a promising way of concentrating the molecules on the sensor surface. Super-hydrophobic artificial surfaces comprising arrays of micro-pillars have been employed for passively confining large biomolecules in diluted solutions to nanophotonic structures, which demonstrated impressive sensitivity performance. However, the studies on much more accurate manipulations of analyte molecules only to the hot spots of nanophotonic structures are still quite limited, which can lead to higher sensor sensitivity.

An objective of the present disclosure is to elucidate the possibilities of addressing the above limitations by designing the advanced nanophotonics structure to achieve the high-performance SEIRA and SERS sensors. The disclosure is composed of seven sections which are organized as follows:

Section 2 reviews the fundamentals of surface-enhanced vibrational spectroscopy. The section begins with an introduction of the molecular vibrations. The basic principles of infrared absorption, Raman scattering, and the related spectroscopy techniques are also discussed. Finally, this section also gives a theoretical discussion of surface-enhanced vibrational spectroscopy and a review of the existing research and development of surface-enhanced vibrational spectroscopy technologies.

Section 3 covers a hybrid metal graphene photonics SEIRA sensor design for stronger graphene optical response as well as the electric field in the terahertz range. The self-aligned photolithography fabrication method is presented to fabricate the uniform nanometric gap size of around 100 nm between the graphene anti-dots patterns and gold core disk. Then the numerical simulation and experiment measurements of the hybrid device are also conducted to demonstrate 3-time enhancement in the extinction of graphene plasmon and almost one order of magnitude increase in the electric field.

In section 4, a nanophotonic SEIRA sensor with the functionality of passive trapping molecules into the hot spots is presented. By optimizing the design of photonics structures, the hot spots (i.e., nanotrenches) of the sensors also play the role of trapping the molecules dissolved in the solution. Then the experiments are conducted to verify the functionality of trapping molecules as well as the nanoparticles (i.e., exosomes) into the nanotrenches. The SEIRA measurements were also conducted to test the sensing performance, which results in the detection from the almost 1 pg level of proline or the glucose.

Section 5 presents a liquid gallium based nanophotonic SEIRA sensor with ultra-high sensitivity. Liquid gallium is used to sandwich the analyte thin film with the metal nanoribbon and form the nanometric gaps as the hot spot of sensors. A simple gallium coating method is present for preparing the sensor device, followed by the experimental SEIRA measurement to confirm the state of art sensing performance with the liquid gallium based sensor. In addition, further simulations were conducted to study the effect of the surface roughness of gold nanoribbons in the sensor and the sensitivity.

In the description above, a low-cost gallium-based SERS sensor is presented. Liquid gallium in the sensor design is used to sandwich the analyte thin film into the nanometric gap which can confine the higher electric field. The numerical simulations conduct a direct comparison of electric field enhancement among the nanostructure such as nanosphere particle, and dimer resonator before and after coating liquid gallium. The preparation procedures of the cost-effective SERS substrate are also given. Finally, Raman spectroscopy measurement with the proposed SERS substrate by coating with BPT showed almost one order of magnitude improvement in the Raman signals and the improvement in the signal spatial uniformity.

### 2. Fundamentals of Surface Enhanced Vibrational Spectroscopy

### 2.1 Molecular Vibrations

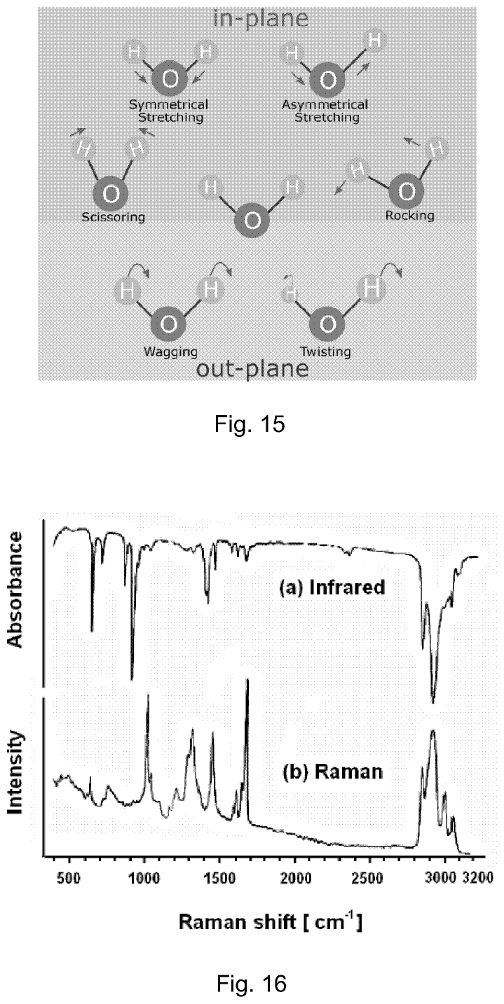

Atoms, the basic composition of the molecules, are always in motion when the temperature is above absolute zero. The periodic patterned movements of the atoms relative to each other in such molecules are called molecular vibrations. Molecular vibrations are really important for exploring the spectral response of materials and understanding the mechanisms and kinetics of chemical reactions. The molecular vibrational movement typically can be pictured as balls connected to the springs. In this sense, the balls represent atoms in the molecule while the coil springs act like chemical bonds. According to the pattern of the movement, the bond vibrations can be summarized as several general types (see FIG. 15), including asymmetrical stretching and symmetrical stretching vibrations, and in-plane and out-of-plane bending vibrations.

Asymmetric stretching vibrational movement happens when atoms travel in opposite directions and the chemical bonds shrink or stretch. In opposite, the atoms move in the same directions for the asymmetric stretching vibrational mode. The movements of both stretching vibrations happen within the same plane.

When the movements of the atoms change the angles between bonds, bending vibrations occur, including scissoring, rocking, twisting, and wagging vibrations. Scissoring vibrations has bonds swing right and left with deformation of the valence angle while rocking vibrations swing bonds right and left in unison. Both scissoring and rocking movements occur in the same plane. As a comparison, twisting vibrations and wagging vibrations change the bonds in the opposite and same out-of-plane direction, respectively. As a result, twisting vibrations and wagging vibrations are the out-of-plane modes.

### 2.2 Spectroscopy of the Molecular Vibrations

Molecular vibrations can interact with electromagnetic waves and leave a vibrational response in the spectra. Under the electromagnetic wave excitation, the molecular vibrations will experience the change of dipole moments and polarizability, which will lead to IR absorption and Raman scattering, respectively. The corresponding spectra can be obtained with IR absorption spectroscopy and Raman spectroscopy respectively. FIG. 16 gives the measured IR and Raman spectrum of styrene-butadiene rubber in which the Raman scattering peaks or IR absorption dips are directly related to the molecular vibrational modes. These vibrational spectra are important since they contain valuable molecular information and can be directly linked to the molecular constituents, their chemical bonds, as well as molecular configuration. Vibrational spectroscopy currently has been widely used in many fields and has played a significant role in both industry and academic institutes in different fields such as chemical analysis, biomolecular analysis, process monitoring, and pollutant detection.

### 2.2.1 Infrared Absorption Spectroscopy

### 2.2.1.1 Basics of Infrared Absorption

When the infrared radiation illuminates on the molecules, the molecular vibrational movement will absorb the infrared radiation at a particular frequency or wavelength. In such a case, there is a change in dipole moment for the vibrations that absorb the IR radiation.

Two molecular properties that are defined by the charge distribution at the equilibrium geometry of the electronic state will change with variations in the internuclear distance (or any of the vibrational degrees of freedom in a polyatomic molecule): the dipole moment u and the molecular polarizability a. The change of dipole moment results in the infrared absorption while the latter one will cause the Raman scattering which we will talk in the following section.

For the dipole moments vector at the equilibrium p=pₓx+p_{y}y+p_{z}z, each of its components can be expanded with the series form:

$$p = {p}_{0} + \frac{\partial p}{\partial d} {d}_{0} cos ( {ω}_{0} t ) + \frac{{\partial }^{2} p}{\partial {d}^{2}} {d}_{0}^{2} {cos}^{2} ( {ω}_{0} t ) + … \qquad ( 2.1 )$$

where p₀ represents the equilibrium value of the dipole moment. The displacement d has the form d(t)=d₀ cos(ω₀t). Since only the harmonic oscillating of the dipole moments will absorb the electromagnetic wave, the first term, the permanent dipole moment p₀, will not appear in the infrared spectrum. If higher order terms are ignored, the infrared absorption intensity will have the relation in the following form:

$${I}_{IR} ∝ {( \frac{\partial p}{\partial d} {d}_{0} cos ( {ω}_{0} t ) )}^{2} \qquad ( 2.2 )$$

We can see from the above relation that infrared absorption will occur only when the vibrational movement causes the change of the dipole moments. Considering that the absorption of an oscillating dipole is proportional to the field intensity of the incident wave, the infrared absorption of the vibrations can be represented in a more general form:

$${I}_{IR} ∝ {E}_{0}^{2} \qquad ( 2.3 )$$

where E₀ is the amplitude of the incident wave. As a result, the absorbed infrared light intensity is proportional to square of the electric field or intensity of incident wave. Since the dipole moment is a vector, incident radiation with the different orientations will excite the change of dipole in different directions and hence the absorption of the material has the orientation dependency.

### 2.2.1.2 Type of the Infrared Absorption

According to the previous section, the dipole moments change in the molecular vibrations will cause the IR absorption. Whether a vibrational movement will contribute to the change of the dipole moments or not can be determined by the symmetry of the vibrational movement. By considering the type of symmetry for the atom's movement, the molecular vibrations will show the behavior of IR absorption active or inactive.

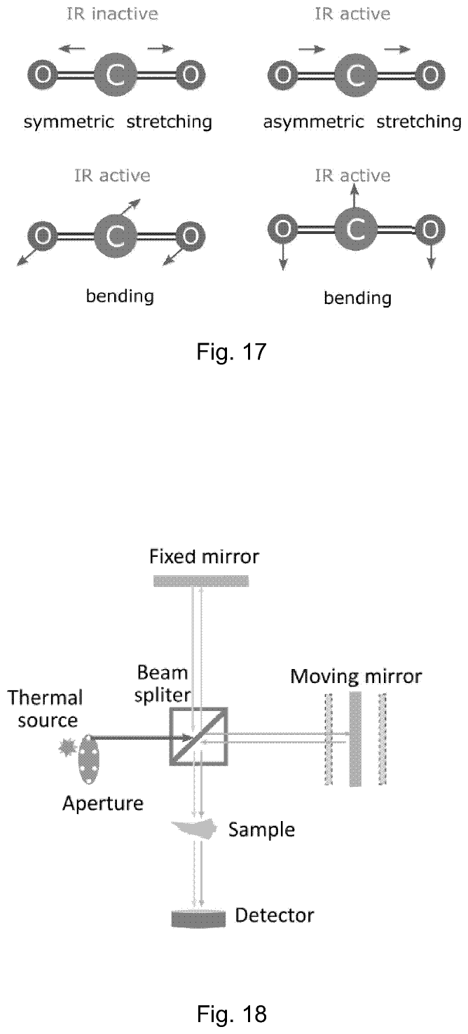

Here we use the carbon dioxide molecules as an example (see FIG. 17). For the symmetric stretching mode, the vibrational movements are central symmetric and atoms movement will not cause the change of dipole moments. Because of this, symmetric stretching cannot result in the absorption of infrared radiation. In another hand, there is a change in dipole moment for the asymmetric stretching mode because of the asymmetric distribution with respect to the central atom. Hence an asymmetric stretch will cause infrared absorptions in the spectrum. Bending motions cause the change of dipole moments as well and hence can result in the absorption of infrared radiation. Generally, it takes more energy to stretch a bond than to bend one, thus stretching vibrations normally occur at higher frequencies

The infrared absorption described above is directly caused by the vibrational modes and hence they are called fundamental vibrational modes. In addition to the fundamental vibrational modes such as stretching and bending modes, there are also some non-fundamental vibrations. These non-fundamental modes are typically excited by higher energy radiations and hence show higher vibrational frequency or lower wavelength. The non-fundamental modes are the results of the mixed excitation of the foundational modes, i.e., overtone and combination. Overtone bands usually occur at the integer multiples of fundamental vibration corresponding absorption frequency. Combination bands result from the coupling of two different fundamental vibrations. These non-fundamental bands can reflect the additional information of the corresponding fundamental vibrations. However, in most cases, the non-fundamental vibrations merely complicate the interpretation of the spectrum.

### 2.2.1.3 Fourier Transform Infrared (FTIR) Spectroscopy

An FTIR system is typically composed of an interferometer for collecting the interferogram and a computer for converting the interferogram to a spectrum.

The interferometer includes four arms (see FIG. 18. The typical configuration of the interferometer in the FTIR system). The infrared light emitted by a broadband source is firstly collected by a collimating mirror to make its rays parallel and then passes the aperture to limit the ray size and control the wave intensity. The infrared wave will propagate to the beam-splitter. A beam-splitter is an optical component that is designed to transmit a portion of the incident wave and reflect another portion of the incident wave. The reflected infrared light from the beam-splitter travels toward a fixed mirror and is reflected to the beam-split again while the transmitted infrared light will travel toward and be reflected by a moving mirror. Then both beams encounter the beam-splitter and combine again with a certain phase difference, where the interference will occur. Finally, the combined beams will interact with the sample and are collected by the infrared detector.

After obtaining the interferogram, the spectrum can be converted with help of the computer. Since in the actual measurement the interferogram is composed of discrete points, the discrete Fourier transform will be used instead of the continuous Fourier transform. For an interferogram I (n·Δx) consisted of N discrete equidistant points, the spectrum (k·Δv) can be obtained from discrete FT by:

$$S ( k \cdot Δ x ) = {\sum }_{n = 0}^{N - 1} I ( n \cdot Δ x ) {e}^{\frac{2 π nk}{N} i} \qquad ( 2.4 )$$

where Δv and Δx are the discrete frequency spacing and position spacing. n and k are the n-th position point and k-th frequency point. The spacing Δv in the spectrum can be related to Δx by:

$$Δ v = \frac{1}{d} = \frac{1}{N Δ x} \qquad ( 2.5 )$$

where d is the total moving distance of the mirror or optical path differences.

We can see that the spectrum resolution is determined by the spatial spacing distance and the total number of collection data points. Typically, FTIR integrates a helium-neon (He—Ne) laser with a fixed wavelength of 632 nm in the optical path of the interferometer and the moving distance of the mirror can be precisely controlled by monitoring the highest signal intensity at the laser wavelength. As a result, interferogram data point intervals are evenly spaced. To increase the resolution, the measurement should collect more data points and hence require greater optical path differences.

### 2.2.2 Raman Spectroscopy

### 2.2.2.1 Basic principle of the Raman scattering

As mentioned in the above section, the excitation of the molecular vibration will result in changes in dipole moments as well as bond polarizability. The change of the polarizability will lead to the Raman scattering process.

The polarizability originates from a deformation of electron cloud after a light interact with the molecules. Here we can analyze the Raman scattering process based on the classical model. An electromagnetic wave E=E₀ cos (ω₀t) with amplitude E₀ and frequency ω₀ interacts with a molecule, the excited oscillating molecular dipole can be expressed as

$$p = α ( {ω}_{0} ) \cdot {E}_{0} cos ( {ω}_{0} t ) \qquad ( 2.6 )$$

where α (ω) is the polarizability tensor. Since the polarizability describes the response of the electron distribution to the movements of the nuclei that oscillate with the normal mode frequency. Hence the polarizability can be expressed with the time dependent form by a Taylor series with respect to the normal coordinates Qu and Ov:

$$α = {α}_{0} + \frac{\partial ∝}{\partial {Q}_{u}} {Q}_{{u}_{0}} cos ( {ω}_{k} t + {δ}_{u} ) + \frac{1}{2} {\sum }_{u , υ} \frac{{\partial }^{2} ∝}{\partial {Q}_{u} \partial {Q}_{υ}} {Q}_{{u}_{0}} {Q}_{{υ}_{0}} cos ( {ω}_{u} t + {δ}_{u} ) cos ( {ω}_{υ} t + {δ}_{υ} ) + … \qquad ( 2.7 )$$

where Qᵤ₀ and Oᵥ₀ are the equilibrium values of the normal coordinates at vibrational frequency ωᵤ and ωᵥ. Combining the equation and ignore the higher order terms, we can obtain

$$\begin{matrix}p = [ {α}_{0} + \frac{\partial ∝}{\partial {Q}_{u}} cos ( {ω}_{k} t + δ u ) ] \cdot {E}_{0} cos ( {ω}_{0} t ) \\ = {E}_{0} {α}_{0} cos ( {ω}_{0} t ) \frac{1}{2} \frac{\partial ∝}{\partial {Q}_{u}} {E}_{0} \\ {Q}_{u 0} [ cos ( ( {ω}_{0} + {ω}_{k} ) t + {δ}_{u} ) + cos ( ( {ω}_{0} - {ω}_{k} ) t - {δ}_{u} ) ] \\ = {E}_{0} [ {α}_{0} cos ( ( {ω}_{0} t ) {α}_{anti} cos ( ( {ω}_{0} + {ω}_{k} ) t + {δ}_{u} ) + \\ {α}_{stroke} cos ( ( {ω}_{0} - {ω}_{k} ) t - {δ}_{u} ) ]\end{matrix}$$

where αₐₙₜᵢ and αₛₜₒₖₑ are the effective polarizability for the anti-Stokes and Stokes Raman scattering. We see that the linear induced dipole moment p has three components with different frequencies. The first term E₀α₀ cos (ω₀t) gives rise to radiation at ω₀ and accounts for the Rayleigh scattering; the second term αₐₙₜᵢ Cos (ω₀+ωₖ) t+δᵤ) gives rise to radiation at ω₀+ωₖ and accounts for the anti-Stokes Raman scattering. The last term αₛₜₒₖₑ cos((ω₀−ωₖ) t−δᵤ) gives rise to radiation at ω₀−ωₖ and accounts for the Stokes Raman scattering.

### 2.2.2.2 Raman Spectroscopy

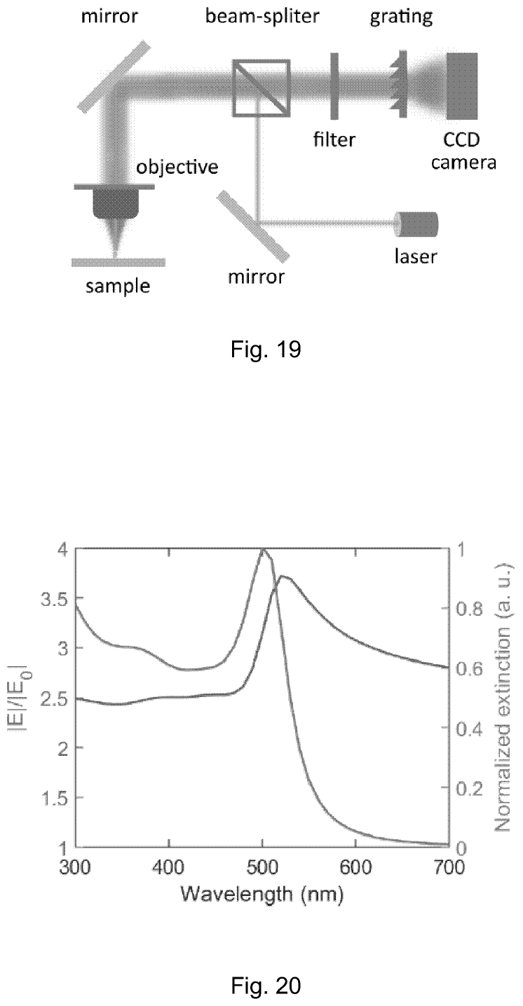

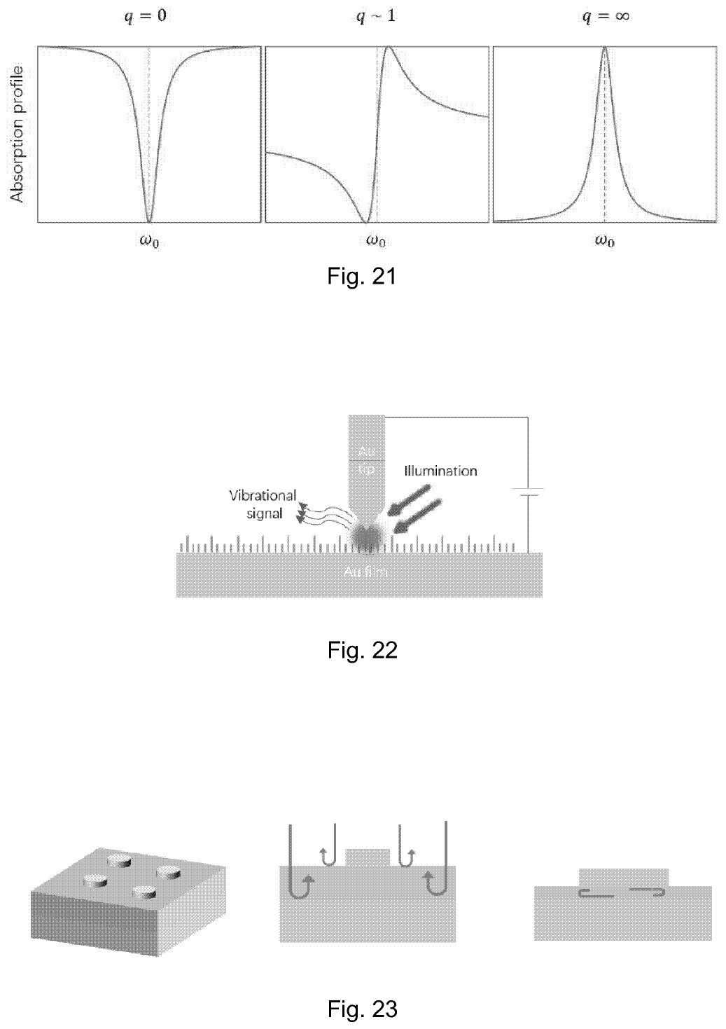

FIG. 19 shows the typical schematic configuration of the Raman spectroscopy. The laser beam is incident on the sample surface through the objective and the scattered radiation is collected by the objective again. The collected scattered light then passes through a filter which removes Rayleigh scattering component of light at the frequency of the laser and allows only frequency-shifted light to pass through. Then the scattered light will be split up into different frequencies traveling in different locations by a grating and are finally detected by the CCD array to yield the Raman spectrum.

There are a few crucial components in a typical Raman system, including laser excitation, collecting optics, filter, and detector.

Laser: A laser illuminated on the sample should have a suitable power, wavelength, and stability. In addition, wavelength of the excitation laser can affect the Raman scattering cross-section with the relation of (σ∝1/λ⁴),

Which indicates that the laser with shorter wavelength will provide higher Raman signal. The most commonly used lasers are in visible regions because of their stable power and frequency, long lifetime, and narrow linewidth. The main problem with visible lasers is that high photon energy will cause fluorescence which is a much stronger process compared with the weak Raman scattering. For this reason, near-infrared lasers at frequencies such as 795 nm or 785 nm are also used to reduce fluorescence from many compounds.

Collecting optics: Since Raman scattering is a relatively weak process, it is important to efficiently collect the scattered light in Raman spectroscopy. The collecting optics typically have a large numerical aperture.

Filter: A filter should be used to remove the Rayleigh scattering component, otherwise the Raman signal would be swamped in the strong Rayleigh scattering signal. Various types of optical narrow-band rejection or edge filters are available to perform this function. There are two basic types, notch filters which remove radiation at the frequency of the laser, and edge filters which remove all light above a certain frequency (i.e., only the Stokes scattering signals are allowed to pass). Edge filters are typically cheaper with a long life but they will prevent anti-Stokes scattering signals from transmitting.

Detector: The scattered radiations are divided into different frequencies by the dispersive grating and hence are detected by the array CCD detector. Each sector of the array CCD detector measures the intensity of a narrow-band component of the scattered light. In this way, it is possible to discriminate each frequency of the scattered light and construct a spectrum. Array detectors are suitable for Raman spectroscopy even with IR excitation wavelengths but the cost of the array detector is expensive. Hence the FT-Raman spectroscopy, which has a single element detector coupled to an FT spectrometer, was also developed to replace the array detectors.

### 2.3 Surface Enhanced Vibrational Spectroscopy Technologies

In 1974, Fleischmann et al. found the Raman intensity of the pyridine molecules adsorbed on rough surfaces of silver experienced a dramatic enhancement. This unexpected finding immediately aroused wide interest and promoted intensive research activities in this field. Van Duyne et al. reported the SERS effect in 1977 and Moskovits attributed the enhancement process to the excitation of the surface plasmons (SPs) at the roughened silver surface. Moreover, it was found that this surface-enhanced effect was also effective in the IR absorption process, i.e., SEIRA.

### 2.3.1 Surface Plasmons

### 2.3.1.1 Surface Plasmon at the Interface

In this section, we consider the electromagnetic wave at the interface between two media with dielectric function ε₁ and ε₂. If we choose the interface to coincide with the plane z=0. We start form the wave equation in both media

$$∇ \times ∇ \times E ( r , ω ) - \frac{{ω}^{2}}{{c}^{2}} {ε}_{j} ( r , ω ) E ( r , ω ) = 0 \\ j = 1 , 2 \qquad ( 2.9 )$$

The general solution for the above equation can be expressed as

$${E}_{j} = {E}_{x , j} [ \begin{matrix}1 \\ 0 \\ - {k}_{x} / {k}_{z , j}\end{matrix} ] {e}^{{ik}_{x} x - i ω t} {e}^{{ik}_{z , j}} \\ j = 1 , 2 \qquad ( 2.1 )$$

By applying the boundary condition, we can obtain the following relation:

$${k}_{x}^{2} = \frac{{ε}_{1} {ε}_{2}}{{ε}_{1} + {ε}_{2}} {k}^{2} = \frac{{ε}_{1} {ε}_{2}}{{ε}_{1} + {ε}_{2}} \frac{{ω}^{2}}{{c}^{2}} and {k}_{z , j}^{2} = \frac{{ε}_{j}^{2}}{{ε}_{1} + {ε}_{2}} \frac{{ω}^{2}}{{c}^{2}} \\ j = 1 , 2 \qquad ( 2.11 )$$

According to Drude model (see Section A below), the metal typically has large negative real permittivity. Assuming that medium 1 is metal and medium 2 is air (i.e., ε₂=1), we will get ε₁ε₂<0 and ε₁+ε₂<0 in the relation equation 2.11, which will lead to real kₓ and hence propagating interface waves along the interface. Therefore, localized modes can exist at a metal-dielectric interface, which are the so-called surface plasmon polaritons (SPPs). If we write the metal permittivity as

$${ε}_{2} = {ε}_{1}^{′} + {ε}_{1}^{″} ,$$

then we can derive the SPPs wavevectpr

$${k}_{x} = {k}_{x}^{′} + {k}_{x}^{″}$$

as

$${k}_{x}^{′} = \sqrt{\frac{{ε}_{1}^{′}}{{ε}_{1}^{′} + 1}} \frac{ω}{c} and {k}_{x}^{″} = \frac{{ε}_{1}^{″}}{2 {ε}_{1}^{′} ( {ε}_{1}^{′} + 1 )} \sqrt{\frac{{ε}_{1}^{′}}{{ε}_{1}^{′} + 1}} \frac{ω}{c} \qquad ( 2.12 )$$

Then the SPPs wavelength can be derived

$${λ}_{SPP} = \frac{2 π}{{k}_{x}^{′}} = \sqrt{\frac{{ε}_{1}^{′} + 1}{{ε}_{1}^{′}}} λ \qquad ( 2.13 )$$

where λ is the wavelength in the free space. We can see now that the SPP wavelength is always shorter than that in the free space especially when the real part of the metal close to −1.

### 2.3.1.2 Localized Surface Plasmon

When a metal nanoparticle rather than the continuous metal interface is considered, a non-propagating SPP will be confined to nanostructured surfaces and a localized surface plasmon (LSP) resonance occurs. SPP can provide strong local field enhancement which can be beneficial for the enhancement of the vibrational signal. However, resonant structures such as an Au nanoparticle can be formed to support the LSP for even higher local field enhancement.

A full theoretical treatment of localized surface plasmons for a variety of nanostructures is quite lengthy. Here, we use the gold sphere as an example to understand the LSP and the local EM field enhancement. The gold sphere in a homogeneous medium with permittivity ε₁ has the radius a and it is irradiated by z-polarized light with the wavelength much larger than the sphere radius. The electric field out of the gold sphere Eₒᵤₜ and the corresponding extinction spectrum under the electrostatic approximation is given by:

$${E}_{out} = {E}_{0} \vec{z} - \frac{{ε}_{Au} - {ε}_{1}}{{ε}_{Au} + 2 {ε}_{1}} {a}^{3} [ \frac{1}{{r}^{3}} \vec{z} - \frac{3 z}{{r}^{5}} \vec{{r}_{m}} ] {E}_{0} \qquad ( 2.14 )$$

and the corresponding extinction spectrum

$$I = \frac{12 π {Na}^{3} {ε}_{1}^{\frac{3}{2}} ω}{c} [ \frac{Im ( {ε}_{Au} )}{{( Re ( {ε}_{Au} ) + 2 {ε}_{1} )}^{2} + {[ Im ( {ε}_{Au} ) ]}^{2}} ] \qquad ( 2.15 )$$

We can see from the above equations that both the electric field out of the gold sphere and extinction spectrum will approach maximum when ε_{Au}+2ε₁ is close to zero (see FIG. 20). In such a case, the LSP resonance can support strong local field enhancement and optical response.

### 2.3.2 Vibrational Signal Enhancement with Surface Plasmons Resonator

### 2.3.2.1 Raman signal enhancement with surface plasmons

As described above, surface plasmons resonance exists in many noble metals surface or metal nanoparticle resonators. Meanwhile, an enhanced electric field is confined at the surface of the metal resonator.

When an incident wave E₀(ω₀) illuminates on the resonator, the local electric field at the meal interface is

$${E}_{loc} ( {ω}_{R} , {r}_{R} ) = {g}_{1} ( {ω}_{R} , {r}_{R} ) {E}_{0} ( {ω}_{0} ) \qquad ( 2.16 )$$

where g₁(ω_{R}, r_{R}) is the enhancement factor of electric field at the resonator surface position r_{R}. The corresponding induced dipole moments for the molecules near the metal surface is

$$p ( {ω}_{m} , {r}_{m} ) = {α}_{m} ( {ω}_{m} , {r}_{m} ) {E}_{loc} ( {ω}_{m} , {r}_{m} ) \qquad ( 2.17 )$$

where αₘ(ωₘ, rₘ) is the Raman polarizability derivatized on molecular vibrational frequency ωₘ at position rₘ. In verse, the induced dipole moments p (ωₘ, rₘ) in the molecules would induce dipole moments p_{R}(ωₘ, r_{R}) in the resonator as well. Under the induced dipole approximation, induce dipole moments in the resonator could be expressed by

$${p}_{R} ( {ω}_{m} , {r}_{R} ) = {μμ}_{0} {ω}_{R}^{2} {α}_{R} ( {ω}_{m} , R ) \cdot G ( R , {r}_{m} ) \cdot p ( {ω}_{m} , {r}_{m} ) \qquad ( 2.18 )$$

where α_{R} (ωₘ, R) is the polarizability of the resonator, G (R, rₘ) is the dyadic Green's function and μ and μ₀ are the relative permeability and vacuum permeability. The total induced dipole moments will be

$$\begin{matrix}{p}_{total} = p ( {ω}_{m} , {r}_{m} ) + {p}_{R} ( {ω}_{m} , {r}_{R} ) \\ = [ 1 + {μμ}_{0} {ω}^{2} {α}_{R} ( {ω}_{m} , R ) \cdot G ( R , {r}_{m} ) ] {p}_{m} ( {ω}_{m} , {r}_{m} ) \\ = {g}_{2} ( {ω}_{m} ) {p}_{m} ( {ω}_{m} , {r}_{m} )\end{matrix} \qquad ( 2.19 )$$

where g₂ (ωₘ) contributes to the radiative enhancement. The total scattered signals detected at far-field will have the following relation:

$${I}_{raman} ∝ {\vert {g}_{1} ( {ω}_{R} ) \vert}^{2} {\vert {g}_{2} ( {ω}_{m} ) \vert}^{2} {\vert {α}_{m} ( {ω}_{m} ) {E}_{0} ( {ω}_{k} ) \vert}^{2} \qquad ( 2.2 )$$

The total gain comes from the resonator will be

$${{G}_{SERS} = {\vert {g}_{1} ( {ω}_{R} ) \vert}^{2} {\vert {g}_{2} ( {ω}_{m} ) \vert}^{2} ( {ω}_{R} ) \vert}^{2} \qquad ( 2.21 )$$

$$where$$

$${\vert {g}_{1} ( {ω}_{R} ) \vert}^{2} = {\vert \frac{{E}_{loc} ( {ω}_{l} )}{{E}_{0} ( {ω}_{k} )} \vert}^{2} , {\vert {g}_{2} ( {ω}_{m} ) \vert}^{2} = {\vert \frac{{E}_{rad} ( {ω}_{l} )}{{E}_{rad , 0} ( {ω}_{k} )} \vert}^{2} \qquad ( 2.22 )$$

If the vibrational frequency shifts are not large (i.e., ω_{R}≈ωₘ), the local field enhancement g₁(ω_{R}, r_{R}) and the radiative enhancement g₂(ωₘ, r_{R}) will be close. Then above relation can be further simplified for as:

$${G}_{SERS} ≅ {\vert {g}_{1} ( {ω}_{R} ) \vert}^{4} = {\vert \frac{{E}_{loc} ( {ω}_{l} )}{{E}_{0} ( {ω}_{k} )} \vert}^{4} \qquad ( 2.23 )$$

This expression is referred to as the zero Stokes shift limit of the |E|⁴ approximation. It is more accurate at small Raman shifts and if the frequency distribution of g₁(ω_{R}) is not too sharp near ω_{R}.

### 2.3.2.2 IR Absorption Enhancement with Surface Plasmon

Similarly, the IR absorption can also be enhanced with the enhanced electric field E_{loc}(ωₖ) near the resonator excited by an incident wave illumination. Combining the equation 2.3 and 2.16, the IR absorption of the molecules near the resonator can be enhanced as:

$${I}_{IR} ∝ {\vert {E}_{loc} ( {ω}_{k} ) \vert}^{2} = {\vert {g}_{1} ( {ω}_{R} ) \vert}^{2} {\vert {E}_{0} ( {ω}_{k} ) \vert}^{2} \qquad ( 2.24 )$$

The total enhancement comes from the resonator will be

$${G}_{SEIRA} = {\vert {g}_{1} ( {ω}_{R} ) \vert}^{2} = {\vert \frac{{E}_{loc} ( {ω}_{l} )}{{E}_{0} ( {ω}_{k} )} \vert}^{2} \qquad ( 2.25 )$$

From the above equation, the enhancement of the IR absorption resulting from the resonator is directly related to the electric field enhancement in the resonator.

In addition to the intensity of absorption, the resonator can also affect the line-shape of molecular absorption. As a typical signature of SEIRA, asymmetric line shapes of absorption are usually observed. This asymmetric absorption line shape results from the coupling between the resonance of nano-structure and the molecular vibration. A few models were proposed to describe this coupling. Here we use the Fano resonance model to explain this coupling. Fano-resonances, which describe the coupling between a broad spectra or continuum (i.e., the resonance of nanostructure) and a narrow or discrete resonance (i.e., the molecular vibrations), were initially found in quantum mechanical systems and can also be applied in classical optical system. The shape of the resonance caused by the coupling can be describe as:

$$I = \frac{{( 1 γ + ω - {ω}_{0} )}^{2}}{{( ω - {ω}_{0} )}^{2} + {γ}^{2}} \qquad ( 2.26 )$$

where ω₀ is the resonance frequency, γ is the width of the resonance, and q is the Fano-parameter depending on the phase of the electromagnetic interaction. A perfect match of phase (i.e., q=0) lead to an anti-absorption (induced transmission) while q=∞ will lead to the symmetric absorption (see FIG. 2.7). As the resonant frequency is detuned with respect to the vibrations (i.e., q≠0 or q≠∞), a clearly asymmetric absorption profile can be observed.

### 2.3.3 Surface Enhanced Vibrational Spectroscopy Technologies

From the above sections, we can see that the enhancement factors of the vibrational signal are directly linked to the local electric field experienced by the molecules. The geometrical and spectral properties of the nanostructures can significantly affect the enhancement of the electric field and hence the design of the photonic resonator is vital for increasing the sensitivity of SERS and SEIRA. Therefore, over the last decades, researchers strived to optimize the geometries and choice of materials for nanostructures, their arrangements, and the experimental measurement configurations to increase the enhancement factor for the SERS and SEIRA sensing applications. In this section, the surface enhancement technologies from the aspect of the structure geometries and material choice will be discussed.

### 2.3.3.1 Non-Resonant Structures

The surface enhanced vibrational technologies of non-resonant enhancement are mainly based on the lightning rod effect, which is that metallic surfaces with sharp curvatures will cause a strong potential gradient and strong field enhancement. However, the electric field enhancement of the non-resonant structure is typically smaller as compared to resonant surface plasmon resonators for the vibrational signal enhancement.

The first demonstration of the SERS and SEIRA was based on the rough gold or silver surface where the surface plasmons in the rough metal surface can support the enhanced electric field. Similar phenomena were also observed in the island nature of the thin noble metal films. For the metal island films, the signal enhancement factor was found to be strongly dependent on the metal surface morphology which can be influenced by the preparation conditions, such as temperature, deposition rate, and surface morphology of substrate. The distribution of particle size and distance between particles are commonly random, which can broaden light frequency response and hence enhance infrared absorptions in a broadband range. However, the random nature of the metal island film makes the local field enhancement spatially varied across the device surface, and hence it may be difficult to do quantitative analysis of the sensing signal. Because metal-island films can be produced in a much easier and cheaper way than the nanolithography methods, they are widely used for surface-enhanced vibrational spectroscopy.

Thanks to the lighting rod effect as well, sharp metal tips can support a particularly large field enhancement at the apex of the tip. Based on this concept, tip-enhanced Raman spectroscopy was proposed and realized (see FIG. 22). In this technique, a sharp tip, as used for scanning tunneling or atomic force microscopy, is brought into close proximity with that part of a molecular sample that is in the focus of the incident laser beam. Usually, the samples are prepared as an ultrathin film on some metallic (e.g., Ag, Au, Cu, Pt, Pd) film. As a result, the Au or Ag nanotip and a metallic substrate form a coupled structure with a nanogap that supports the LSP with strong local field enhancement. The molecules near the metallic tip experience an enhancement of the Raman scattering or infrared absorption.

In addition to the field enhancement from the surface plasmons, interference is another alternative way to increase the electric field. Sencer Ayas et al. proposed the Universal Uniform Electromagnetic Enhancement by using the destructive interference with the metal ground plane. Although this is a cost-effective method, the field intensity enhancement is still limited to be 4 times. Nan Zhang et al. proposed to further increase the field enhancement of the metal island film by introducing the interference ground plane to improve the wave coupling efficiency and hence achieved enhanced SERS sensing performance.

### 2.3.3.2 Resonant Structures

In contrast to the very broad excitation spectra of random metallic nanostructure ensembles such as metal-island films, sharp plasmonic resonances can couple the incident wave much more efficiently and also confine the light field with much higher enhancement, which are suitable for achieving SEIRA and SERS sensing with ultrahigh sensitivity. In this section we will introduce a few commonly used high performance resonators.

Single-object resonator: The most commonly used single object resonator is the nanorod antenna because of its good performance and simplicity. A dipolar mode in a nanorod antenna can be easily excited and the enhanced electric field is confined at the tips of the rods. Nano-rod resonator was firstly used in the experiments for SEIRA by Neubrech et al. in 2008 and then a few following works for the sensing applications were demonstrated with the nano-rod resonators as well. According to the Babinet's principle, the similar optical response in the far-field of the inverse nanostructures can be predicted based on that of their counterparts. But there will be a major difference in the near-field distribution of a nanoantenna and its counterpart. Huck et al. demonstrated that the nano-slot resonator, the counterpart of nanorod, can provide approximately 3 times higher absorption enhancement for the molecules adsorbed on the sidewalls compared with that of the nanorod resonator. In addition to the nanorod resonators, other similar antenna resonators such as the nano-disk, nano-cubes, triangular nanoprisms, and other multi-branched nanostructures, were also demonstrated for highly sensitive SEIRA or SERS.

Split-ring resonators (SRRs) are another type of the resonant structure established for sensing applications. SRRs are compact nanoring with a nanometer-sized gap, providing a larger near-field enhancement inside the gap region and hence contributing to higher signal enhancements. Also, the configuration of asymmetric SRRs results in the combination of a symmetric (bright) mode and an antisymmetric (dark) mode and hence the formation of Fano-resonance, leading to a ultrahigh Q-factor and electric field enhancement.

Metal-insulator-metal (MIM) resonator: If nanostructures are supported by a dielectric spacer layer and a metallic ground plane, the MIM type resonators, also known as absorber type resonators, are configured. Metal ground plane reflects the incident wave and the reflected wave can destructively or constructively interfere with the incident wave. The thickness of the spacer dielectric layer can affect this interference process by tuning the phase of the reflected wave. When the thickness of the dielectric layer is slightly smaller than a quarter of the wavelength, the optimum enhancement of the electric field occurs and the near-field intensity can be increased by nearly an order of magnitude compared with the nanostructures without the support of the metal ground plane. Besides, the scattering cross-section of the nanostructure above the metal ground plane will affect the additional enhancement of the near field. Brown et al. demonstrated that the large nanostructures with large scattering cross-sections, such as fan-shaped or bowtie antennas, provide more enhancement than that of structures with smaller scattering cross-sections such as thin rods.

Nano-patch antenna is a special case of the MIM resonator with ultra-strong field enhancement. Compared to the MIM absorber with vertical interference optical path, nano-patch antenna has the ultra-thin spacer layer (i.e., down to sub 10 nm) and the horizontal resonant cavity will be formed (see FIG. 23). Stronger interaction will occur between the top nanostructure and the metal ground plane, which supports the strong electric field in the gap between the nanostructure and the ground metal plane. As a result, a variety types of nano-patch antenna are widely studied and used for high performance SERS and SEIRA sensing.

Coupled nano-structures: To achieve higher electric field enhancement, coupled nano-structures with controllable inter-particle nanogaps were extensively investigated, such as dimer resonator, bowtie antenna or the colloidal super-crystals. The electric field in the gap between metal nanoparticle is ultra-strong due to the strong electromagnetic wave coupling. McMahon et al. demonstrated electric field in the gaps between closely spaced nanostructures strongly depends on the gap size g following the relation |E_{loc}/E₀|²∝1/g^{p} with p≈1.2˜1.5. Followed by this principle, a variety of resonators with ultra-small gap are designed and fabricated. With the help of the atomic layer deposition and etching, the gaps in the resonators can be well controlled down to sub 10 nm and over 104 times electric field intensity enhancement can be achieved. Halas and coworkers fabricated the bowtie resonator with a nanogap ˜3 nm and hence obtained the enhanced absorption signal from around 500 molecules. Although the electric field in nanometric gaps is relatively high, it becomes more difficult for the molecules to get into such small gaps and hence the overall molecular vibrational signal enhancement is lower than the enhancement factor of electric field intensity.

Recently plasmonic superlattices prepared with the self-assembled method have been employed as high-performance substrates for enhancing SERS and SEIRA signals. Compared with the island metal film, self-assembled metal superlattices can provide a highly organized photonic structure with small separation gaps and hence a highly uniform electromagnetic field enhancement can be excited in such a reproducible geometry. Liz-Marzan and coworkers assembled nearly perfect three-dimensional super crystals of Au nanorods as the SERS substrates with uniform electric field enhancement, leading to a reproducible high enhancement factor. The synthesized the three-dimensional super-crystals by Niclas S. Mueller et al. induced electromagnetic near-field enhancement all the way from the visible to the mid-IR and provide an excellent enhancement for both SERS and SEIRA sensing applications. They found also found that in addition to the enhancement from the lightning rod effect and the coupling interaction between neighboring nanoparticles, the three-dimensional super-crystal acts as an open cavity resonantly trapping light in the form of standing waves.

### 2.3.3.3 Choice of Materials

In addition to the noble metal such as gold or silver, other kinds of plasmonic materials were also used in the vibrational sensing application and achieved different sensing performances compared with the traditional noble metal.

Aluminum: Compared with gold and silver, aluminum has high optical loss but still is an alternative plasmonic material considering its low cost, and high natural abundance. Aluminum has a high plasma frequency (15.2 eV) and aluminum-based nanostructures can be tuned across the ultraviolet (UV)-visible-near-infrared (NIR) region of the spectrum, which makes the aluminum as a good candidate for the color printing, deep UV resonator and SERS sensing. Since Raman spectroscopy in the UV region shows higher scattering efficiency with the relation of (σ∝1/λ⁴), aluminum-based nanostructures has been well studied for the SERS sensing in the UV region, which shows the ultrahigh sensitivity. L. Li et al. demonstrated an aluminum bowtie nanoantenna for SERS sensing application excited with 258.8 nm laser in the DUV region and achieved a gain of ˜105 in signal intensity from the near field enhancement. S. K. Jha et al. reported a deep UV SERS sensor with aluminum tapered-cylinder array nanostructures and showed an extremely high detection sensitivity of ˜50 zmol adenine molecules.

In ambient conditions, a thin self-passivating oxide layer of about 2-4 nm thickness will be formed on the aluminum surface and hence aluminum keeps stable. In addition, this thin oxide layer makes the resonator's surface suited for a variety of attachment chemistries with various functional groups, which can lead to the wide use of aluminum in biochemical and chemical sensing applications. Benjamin Cerjan etc. reported self-calibrating SEIRA sensors by using aluminum antennas. By calculating the ratio between the enhanced signal strength of the attached molecular vibrations and surface oxide layer vibrations, the authors can obtain information on the molecular coverage and offers an elegant way to determine the number of molecules present on the nano-antennas.

Gallium: Liquid metals combine uniquely the fluidic and conformal nature of liquids with the desirable properties of metals, making them a special branch of plasmonic materials. The commonly used liquid metal include mercury (Hg), alkali metals (e.g., potassium, rubidium), gallium (Ga) and Ga-based alloys. Mercury is traditionally the most recognized liquid metal with a melting point well below room temperature (melting point: −38.8° C.). However, due to the high toxicity, high vapor pressure, and low boiling point of mercury, it is not suitable for many applications. On the other hand, in recent years liquid gallium and its alloys emerged as safe and promising materials for many applications. Gallium has a melting point of 29.8° C. and can stay in the liquid state over a wide temperature range. Liquid gallium has extremely low vapor pressure near room temperature and hence does not evaporate. In ambient condition, an around 2 nm oxide layer is formed on the gallium surface and prevents the bulk gallium from further oxidizing. Gallium has a high bulk plasma resonance energy of 13.9 eV. Compared with gold and silver, this high plasma frequency makes gallium suitable for visible and UV plasmonics applications.

Currently, surface enhanced vibrational spectroscopy mainly focuses on the gallium nano-particles due to its simplicity of the preparation methods and wide range of the LSP tunable range. The gallium nanostructures can be prepared with the top-down methods such as sonication, and bottom-up fabrication techniques including self-assembly during molecular beam epitaxy (MBE), thermal evaporation, and colloidal synthesis. Pae C Wu et al. started to use gallium nano-particle prepared with MBE method as an alternative choice of the gold or silver nano-particle for the SERS sensing application. In the following study, gallium nanoparticles prepared with a low-cost sonication method were decorated with gold grass and then used for SERS detection of R6G, resulting in a limit of detection (LOD) of 10⁻⁷ M. Xin Gao et al. found that narrower size distribution of gallium particles will lead to the much higher signal enhancement. The gallium nanoparticles prepared by the galvanic replacement reaction method had a size regulated LSP resonance behavior and hence achieved 2 orders of magnitude higher than the particles obtained from sonication.

Besides, the propagation length of SPPs in liquid gallium can be increased because of its inherently smooth surfaces and improved optical properties compared to solid Ga due to washing out of inter-band transitions. The unique fluidics property of the liquid gallium can also enable the stretchable and tunable phonics resonator. However, the current study on the liquid gallium for infrared photonics devices is still under development. In the following sections, we will introduce our photonics nano-structure by using liquid gallium for surface-enhanced vibrational spectroscopy sensing application.

Graphene: Since graphene was experimentally discovered by Andre Geim and Konstantin Novoselov in 2004, this monolayer of carbon atoms arranged in a honeycomb lattice has aroused profound interest from researchers due to its appealing electronic, mechanical as well as optical properties. Graphene is also a promising material for mid-IR plasmonic applications. The intra-band transitions of electrons, which are the dominant processes in the mid-infrared and terahertz range, provide graphene with a Drude-like optical conductivity. Compared with other metal-based plasmonics, one of the remarkable properties of graphene surface plasmons is that the compression of the surface plasmon wavelength Apl relative to the excitation wavelength λ₀ can be exceedingly large and this relation can be described as

$$\frac{{λ}_{l}}{λ} \approx \frac{2 α}{{ε}_{1} + {ε}_{2}} \frac{{E}_{F}}{ℏω} \qquad ( 2.27 )$$

where α=e²/(4πℏε₀)=1/137 is fine-structure constant and Er is the Femi energy level. ε₁ and ε₂ are the permittivities of the media on the both sides of the graphene. For the plasmon wavelength in graphene in the mid-infrared range, the above relation typically predicts two orders of magnitude wavelength confinement compared with the excitation wave. In Daniel Rodrigo's work, they estimated that over 90% of the mode energy in the graphene plasmons is confined within a 15 nm distance from the graphene surface, whereas the same percentage spreads over a distance of 500 nm away from the gold surface when surface plasmons of a gold nanostructure are excited. This extremely large electromagnetic-field confinement of graphene plasmonics will be beneficial for achieving superior sensitivity in the detection of large molecules such as proteins, lipids, and DNA.

However, the extreme optical confinement typically leads to low efficiency for the excitations of graphene plasmons. This is manifested by the relatively low measured extinction spectrum of a graphene plasmonic resonance. To overcome this limitation, we proposed the hybrid metal-graphene structure to enhance the plasmonic response as well as the electric near field intensity, which will be described in detail in the following section.

Another promising advantage for the graphene plasmons is that the plasmon frequency of graphene can be electrically tuned. Compared to Raman spectroscopy which uses the laser with a fixed wavelength as the light source, IR spectroscopy usually needs to cover a wide spectral range of infrared light. Dynamically tunable graphene plasmons are highly desirable for SEIRA as well as a wide range of photonics and optoelectronics applications.

### 3. Graphene Anti-Dot Terahertz Plasmonic Metasurfaces Employing Self-Aligned Metal Cores for Sensing Applications

### 3.1 Introduction

Thanks to its various advantageous optical and electrical properties, graphene has recently been employed to develop a variety of nanophotonic devices such as modulators, photodetectors, sensors, and light sources. In particular, graphene supports surface plasmons with highly tunable optical responses in the mid-IR to terahertz (THz) range. Moreover, graphene surface plasmons (GSPs) feature deep subwavelength field confinement and thus extremely strong near-field enhancement and light-matter interactions, which offer great opportunities in sensing and nonlinear-optics areas. As monolayer graphene produced by chemical vapor deposition (CVD) becomes widely available, a variety of tunable plasmonic structures based on patterned graphene have been demonstrated and extensively studied, including arrays of graphene ribbons, disks, and antidots. These graphene plasmonic structures, which are essentially highly tunable metasurfaces, form a suitable platform for surface-enhanced infrared absorption spectroscopy and have been demonstrated to have great potential for quantitative protein detection and molecule identification. However, partly because of the limited carrier density tuning range and mobility of CVD graphene, the resonant optical responses of the experimentally demonstrated graphene plasmonic structures are not as strong as those of metasurfaces having typical metallic resonators. For example, the transmission extinction at the resonance of various monolayer graphene plasmonic structures is usually below 10%. This is especially the case for graphene anti-dot arrays, which exhibit a transmission extinction of below 3%.

Therefore, effective approaches to enhancing the optical responses of graphene plasmonic structures need to be developed, so that the capability of GSPs for mediating strong light-matter interactions can be utilized in a broader range of applications. One of the effective approaches is to employ hybrid metal-graphene nanophotonic structures. Incident light can couple strongly to metallic antennas and be confined to subwavelength scale, which will, in turn, excite GSPs in graphene structures more efficiently. Recently, a variety of hybrid graphene-metal structures with strong and tunable optical responses have been demonstrated.

In this section, we experimentally demonstrate a type of hybrid graphene-metal metasurface that employs gold disks inserted into graphene antidots to drastically enhance the strengths of the tunable graphene plasmonic resonances in the THz range. The resonant transmission extinction of such hybrid meta-surfaces is enhanced by ˜3 times compared to that of bare graphene antidote metasurfaces. In addition, our simulation shows that the metallic core leads to an order of magnitude enhancement of the near-field intensity of the resonant graphene plasmonic modes. Another advantage of our hybrid structure designs is that the minimum feature size of ˜100 nm is conveniently realized with a self-aligned photolithography-based fabrication process that has minimal complexity increase compared to the fabrication of bare graphene antidot arrays. This section is a slightly modified version of published in ACS Applied Nano Materials.

### 3.2 Device Design and Fabrication

### 3.2.1 Design Rationale of the Hybrid Device

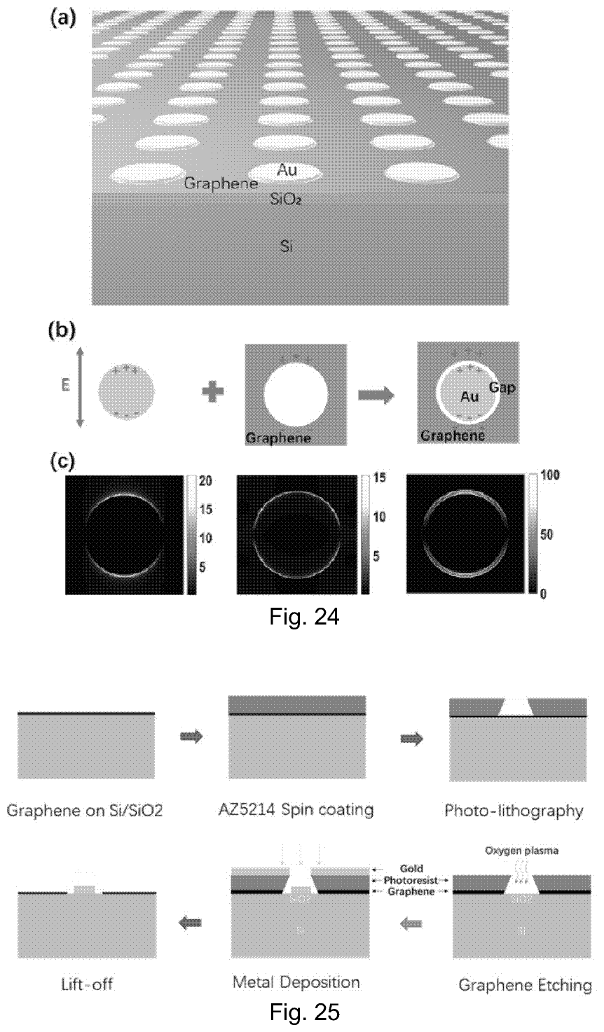

FIG. 24(a) shows the schematic of hybrid structure design, which is based on a graphene antidot array plasmonic metasurface. A thin gold disk is inserted into each graphene antidot unit cell with a small (˜100 nm) gap between their edges. The gold disk essentially functions as a THz optical antenna that can significantly concentrate the electric field (and energy) of an incident THz wave near the thin edges of the gold disk in the subwavelength scale (see FIGS. 24(b) and (c)) and in a broad frequency range. This is also known as the lightning-rod effect. Such enhanced near-fields near the gold disk edges, in turn, lead to more efficient excitation of graphene antidot plasmonic resonances, hence improving the overall optical response of the hybrid structures. An important advantage of this design strategy is that the field enhancement due to the lightning-rod effect occurs in a broad spectral range and therefore is not limited by the resonance frequency of the metallic optical antenna or the plasma frequency of the metal. It can be seen from FIG. 24(c) that the hybrid structure indeed exhibits a significantly larger near-field enhancement compared to the bare graphene antidot or the gold disk alone. As discussed below, the gap size is one of the critical parameters determining the performance of these hybrid structures. Si/SiO₂ substrate was used for these hybrid metasurfaces in which the doped Si functions as a back gate for tuning of the graphene carrier density.

The gap size between the graphene edge and the gold disk edge is a one of critical factors determining the device performance. Due to the lighting rod effect, decreasing gap size will lead to higher electric field which will hence provider higher enhancement factor for sensors. In addition, the gap size can also enhance the extinction response of GSPs due to higher field concentration (see section 3.3.4). In another hand, it will become challenging for reliable mass product and fabrication of device if the gap size is too small (below 50 nm). Considering these trade-offs, we determine the optimal gap size to be in the range of 50 to 100 nm for our experimental demonstration of such hybrid metasurface designs, which can readily achieve one order of magnitude enhancement of the field intensity.

### 3.2.2 Device Fabrication and Characterization

FIG. 25 shows general fabrication procedures of hybrid graphene antidot device. CVD-grown graphene is transferred on the SiO₂/Si substrate. Then the graphene anti-dots are patterned by using a reversal photolithography process and followed by oxygen plasma etching. With the patterned photoresist left on the sample, we deposit Ti/Au (thickness 5 nm/50 nm) to form a metallic disk in each etched graphene holes. After lift-off, graphene anti-dots containing self-aligned gold disks are realized. A second step of photolithography followed by metal deposition and lift-off defines the electrodes for the patterned graphene. All the fabricated structures have a surface dimension of 3 mm by 3 mm. The following sections give much more detailed descriptions of the fabrication procedures.

### 3.2.2.1 Graphene Transfer

CVD-grown graphene on copper foil was used for realizing the hybrid devices. Copper foil with CVD graphene is spin-coated with a poly (methyl methacrylate) (PMMA) film at 3000 rpm and then baked at 180° C. for 2 minutes. After etching the bottom side graphene with oxygen plasma, the copper foil is etched by floating the PMMA/graphene/copper samples on an ammonium persulfate (0.1 M, Sigma-Aldrich) solution for about 1 hour. The samples are subsequently rinsed in deionized water several times, and then transferred onto the target SiO₂/Si substrate and baked at 150° C. for 20 minutes on a hot plate to increase the bonding to the substrate. The PMMA layer is then dissolved in acetone and the samples are cleaned in isopropyl alcohol and blown dry.

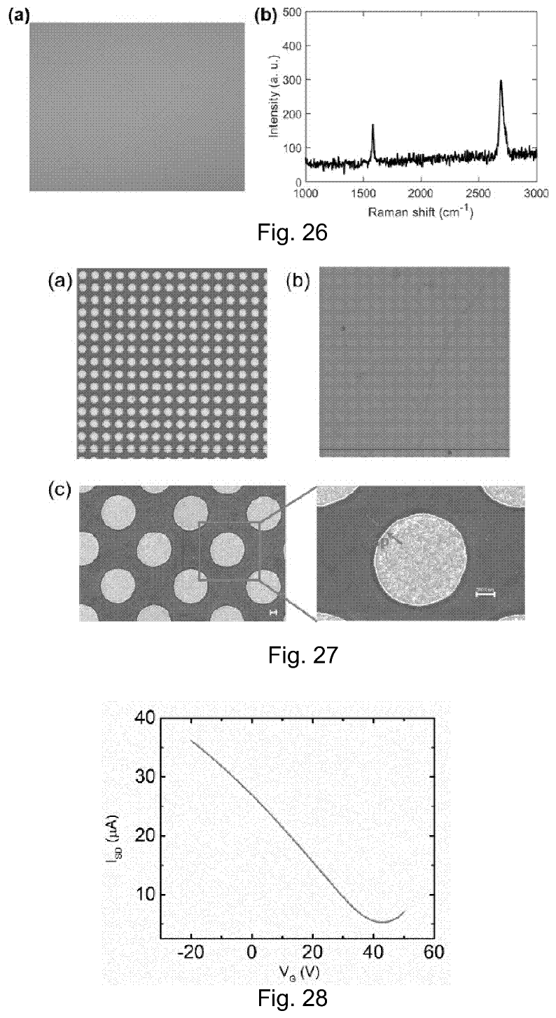

The transferred graphene was characterized by optical images and Raman spectroscopy. FIG. 26 shows the optical image token from the optical microscope with the 100× objective. The graphene surface looks clean and the grain boundary can be observed. The measured Raman spectroscopy spectrum is shown in FIG. 26. Both G-band (1585 cm⁻¹) and 2D band (˜2700 cm⁻¹) of the graphene can be clearly observed and the Raman intensity ratio of 12D/IG is close to 2, which indicates the good quality of single layer graphene. The missing of the D-band in the spectrum also indicates the low defect on the transferred graphene.

### 3.2.2.2 Fabrication of the Graphene Antidot with Metal Core

Because the gaps between graphene edge and metal cores are designed as small as possible (i.e., ˜100 nm) to achieve better device performance (see section 3.3.4), the typical method to realize such a small gap is electron-beam lithography (EBL). However, it would be still challenging to reliably fabricate a large-area (millimeter scale and above) metasurface employing such a hybrid unit cell using a two-step EBL process. Therefore, we develop instead a one-step self-aligned photolithography-based process that proves to be capable of realizing the small gaps with satisfying accuracy across a large area, without introducing significant fabrication complexity compared to the fabrication of bare graphene antidot arrays. As illustrated in FIG. 25, antidots of a reversible photoresist (AZ5214E) are first patterned on the graphene using photolithography so that an undercut profile of the photoresist sidewall is formed. After etching graphene with oxygen plasma to form graphene antidots, Ti/Au (5 nm/50 nm) is deposited on the sample surface using an electron-beam evaporator, and hence self-aligned gold disks are formed in the graphene antidots. FIGS. 27(a) and (b) show the optical images of fabricated graphene antidots devices with and without the metal cores, respectively. The undercut profile of the photoresist sidewall leads to a small gap between the edge of the gold disks and the edge of the graphene antidots. Because the undercut profile of the photoresist depends on parameters including the thickness of the photoresist and the exposure dose, we can control the gap size to some extent by fine-tuning these relevant process parameters. FIG. 27(c) shows a scanning electron microscopy (SEM) image of a small region of a fabricated device, in which the patterned graphene and the periodic gold disks are clearly seen. In the zoomed image in FIG. 27(c), the gap is observed to vary typically in the range of ˜70-100 nm and can be as large as ˜120 nm at some locations. According to the simulation, such a gap-size variation is expected to cause inhomogeneous resonance broadening and lower resonance enhancement factors. Nevertheless, significant enhancement of the GSP resonances should be achieved with such a gap-size variation range.

### 3.2.2.3 Electrical Characterization of Graphene Structures

After the fabrication of the graphene patterns, the metal electrode connected with the graphene sheet is fabricated with the second step of photolithography to conduct the following electrical characterization of graphene. Since the fabricated devices are essentially graphene field-effect transistors (GFET), the electrical properties of graphene channels in GFET can be derived by measuring the transfer curves. The transfer curves are obtained with the devices placed in vacuum at room temperature, and we used a Keithley 2614B Dual-channel Source-meter for the electrical measurement. FIG. 28 shows a typical transfer curve of hybrid graphene devices. The charge neutral point is at positive gate voltages, which shows that the graphene is p-doped, likely due to chemical residues from the fabrication process.

We estimated the carrier mobility from the device transfer curves. Here we used a fitting method to extract the mobility. The total resistance of the GFET is the combination of the contact resistance between graphene and metal and the channel resistance:

$${R}_{total} = {R}_{contact} + {R}_{channel} = {R}_{contact} + \frac{L}{W} \frac{1}{e μ \sqrt{{{n}_{0}^{2} + n \{ Δ {V}_{G} ]}^{2}}} \qquad ( 3.1 )$$

where L and W represent the length and the width of the channel, u is the carrier mobility of graphene, e is unit charge, no is the residual charge density while n [IG] is the gate dependent carrier density which can be expressed as en [ΔV_{G}]=Cₒₓ|V_{G}−V_{CNP}|. By fitting equation 3.1 to the experiment results, we can obtain the value of R_{contact}, μ and n₀.

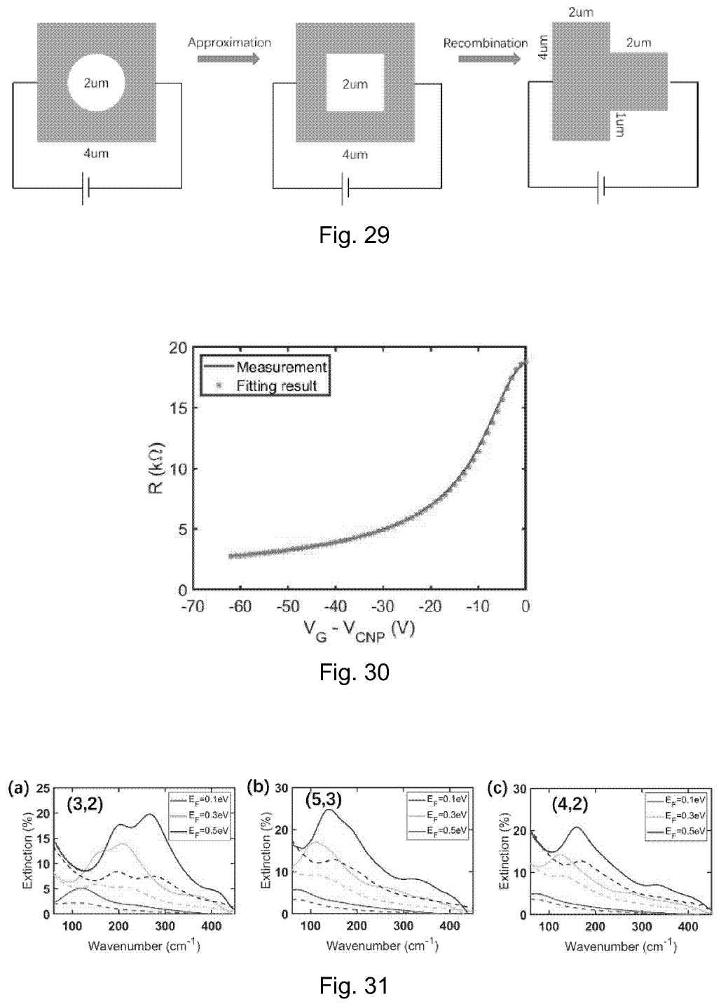

In view of the fact that patterned graphene is not a continuous sheet, we cannot directly use the length and width of the entire graphene area. However, the equivalent circuit of graphene anti-dot structures can be approximated following the schematic shown in FIG. 29. We use the graphene anti-dot (4, 2) as an example in FIG. 29, and determine the (approximate) effective ratio L/W of a single unit cell to be 3/2. Since the width and the length of the metasurface structures are equal (3 mm), the effective ratio L/W of the entire structure is also 3/2, which can be applied to equation 3.1. FIG. 30 shows the channel resistance dependence on the gate voltage. By fitting this curve using equation 3.1, we obtained the following fitting results: mobility μ=928 cm² V⁻¹ s⁻¹, contact resistance R_{contact}=620Ω, and residual charge density n₀=5.3×10¹¹ cm⁻².

### 3.3 Simulation Results

### 3.3.1 Numerical Simulation Configuration

The optical responses of the reported structures were simulated using a commercial software (Lumerical FDTD) which is based on the Finite Difference Time Domain method. A broadband normal incident plane wave is used as the source. The near filed monitor is located at 5 nm above the patterned graphene surface and the transmittance monitor is 30 μm blew the graphene surface. The relative permittivity functions of silicon and silicon dioxide are obtained from tabulated data1. The relative permittivity function of gold is based on the Drude model with plasma frequency 2.17×10¹⁵ Hz and collision frequency 1.5×10¹³ Hz. We modelled graphene as a 2D conductive layer with a Drude-like conductivity function. The reference was obtained by setting the Fermi level of the patterned graphene to 0 eV. Simulations with graphene at different Fermi levels were then conducted and we obtained the transmission extinction spectrum as 1−T/T_{ref}.

### 3.3.2 Simulated Extinction Spectrum Enhancement

FIG. 31(a)-(c) show the transmission extinction spectra of three examples, in which the index (p, d) represents the periodicity p and the diameter d, respectively, of the graphene antidots arranged in a square-lattice configuration. The gap between the gold disk edge and the graphene edge is set as 100 nm, which is an optimized parameter (as discussed later). In each figure, the transmission extinction spectra of each hybrid structure at various Fermi levels are compared with those of the bare graphene antidot array with the same geometric parameters (i.e., periodicity and diameter). All structures exhibit clear GSP resonances at different frequencies in the THz range; however, the common feature of all of the designed hybrid structures is that the transmission extinction due to the GSP resonances is enhanced by 2-3 times compared to that of the corresponding bare graphene antidot arrays. There is also a small red shift of the GSP resonance peaks for the hybrid structures, which can be attributed to the reduction of the restoring force for GSP oscillation as a result of image charges forming in the gold disks (i.e., screening of the GSPs by the gold disks). The THz resonance peaks of all the structures exhibit significant frequency tuning by about 100 cm⁻¹ (3 THz) as the Fermi level varies from 0.1 eV to 0.5 eV. The frequency tuning follows the scaling law ω

$$∝ {n}^{\frac{1}{4}} ∝ \sqrt{{E}_{F}} ,$$

which is a unique characteristic of GSPs.

### 3.3.3 Simulated Electric Field Enhancement

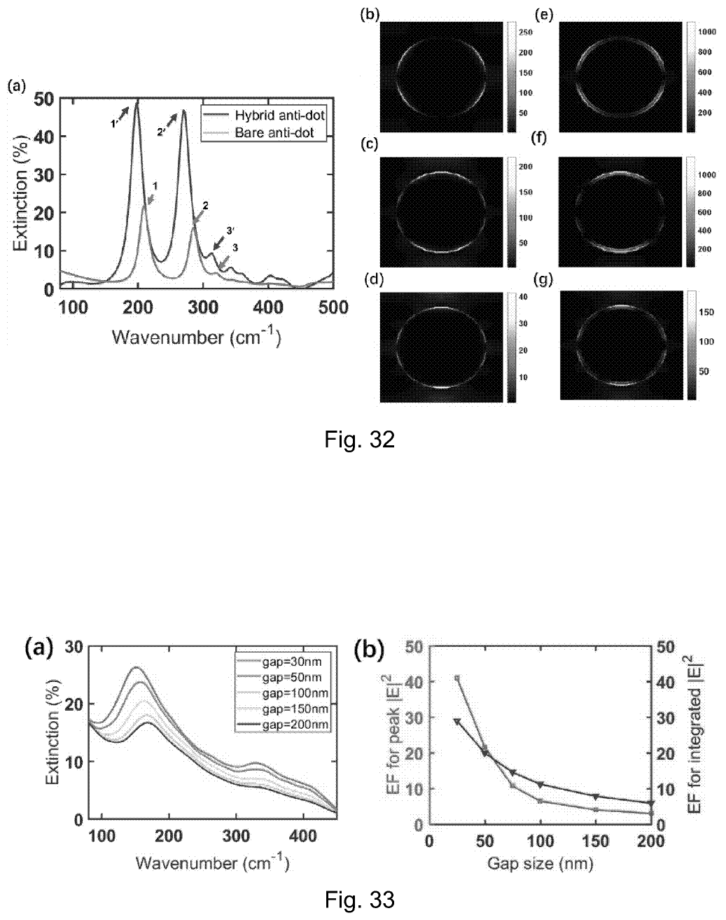

To better understand the observation that some higher-order modes are clearly visible only in the spectra of the hybrid structures, we further simulate the hybrid structures assuming a lower carrier scattering rate (corresponding to 1 meV in energy) in order to obtain sharper resonance peaks, which makes it easier to analyze their relative enhancement due to the gold disks. In the simulated transmission extinction spectra shown in FIG. 32, sharp resonance peaks of different modes are clearly observed. Enhancement of the transmission extinction at the resonance peaks of the hybrid structure with respect to those of the bare graphene antidot structure is different for the three modes marked in FIG. 32(a), which are calculated to be 2.2, 2.9, and 2.3, respectively. Because the enhancement (for both transmission extinction and near-field intensity) is partially determined by the overlap between the field profile of the gold disk (FIG. 24(c)) and those of the resonant GSP modes of the graphene antidots (FIG. 32(b)-(d)), the second resonant mode in FIG. 32(a) exhibits a larger enhancement factor as a result of its field profile, matching that of the gold disk better.

Despite the large field-intensity enhancement, the near-field profiles of all of the resonant GSP modes are not significantly altered by the presence of the gold disks, as can be seen in FIG. 32(e)-(g) in comparison with FIG. 32(b)-(d). It is also interesting to note that the transmission extinction of even higher-order resonant GSP modes of some graphene antidot geometries can be enhanced by even larger factors (see Table 3.1). Because of higher spatial confinement, these even higher-order GSP modes couple weakly with incident plane waves. However, the highly confined and enhanced near-fields of the gold disks can excite these higher-order GSP modes more effectively and efficiently, and hence the enhancement factors can be more significant.

TABLE 3.1
The calculated extinction peak intensity EF for different modes

| Structure | 1st | 2nd | 3rd | 4th |
|---|---|---|---|---|
| Index | mode EF | mode EF | mode EF | mode EF |
| (3, 2) | 2.24 | 2.94 | 2.28 | 2.4 |
| (4, 2) | 1.87 | 1.67 | 2.52 | 2.95 |
| (5, 3) | 2.08 | 2.2 | 2.66 | 4.15 |

### 3.3.4 Gap Size Dependence of Near Field Enhancement

The gap size is an important parameter in our hybrid device design and it plays a critical role for the device performance. The systematic simulation of HGAD device (4, 2) with various gap size are conducted to determine its optimal value. FIG. 33(d) shows the transmission extinction spectra of a hybrid structure design assuming various gap sizes. With the decrease of the gap size, the GSP resonant peaks of extinction spectrum experience the red shift and the intensity of the resonant peaks become stronger. With smaller gap sizes, the field concentration of gold disks due to stronger lighting rod effects leads to a greater enhancement of GSP resonances as well as a greater screening (frequency red-shift) of the GSP resonances.

The simulated gap-size dependence of the enhancement factor (EF) of the near-field peak intensity of the hybrid structures is shown in FIG. 33 (the red curve). The near-field peak intensity EF exponentially increases with decreasing gap size and the peak intensity EF is over 40 with a gap size of 25 nm. The EF is around 7˜ 21 for the gap size in the range of 50 nm˜100 nm, which is close to the gap size variation range in our experimental realization. As the gap size decreases, the area of high-intensity near-field (mostly in the gap region) decreases as well. For applications such as molecular sensing, the integrated field intensity over the entire device surface area is a more useful quantity to compare and optimize. Therefore, we also calculate the EF of the surface-integrated field intensity as a function of the gap size, which is also plotted in FIG. 33 (the blue curve). This EF also increases significantly with decreasing gap size, although its increase is slower than the EF of the peak intensity (the red curve).

### 3.4 Experimental Results

### 3.4.1 Measurement Configuration

We fabricated several hybrid graphene-metal metasurfaces and characterize their transmission extinction spectra at various graphene carrier densities (back-gate voltages) using a Fourier transform infrared spectrometer. The optical measurements were performed using a Bruker Vertex 70v Fourier transform infrared spectrometer which has a vacuum sample chamber. We first measured the transmission spectrum of any given device at the graphene charge neutrality point (TCNP) as the background. The transmission spectra of the device at varied Fermi levels (TEF) were then measured, and the transmission extinction spectra were calculated as 1−T_{EF}/T_{CNP}. The integration time for each transmission spectrum was 10 minutes.

### 3.4.2 Optical Response of Gold Disk

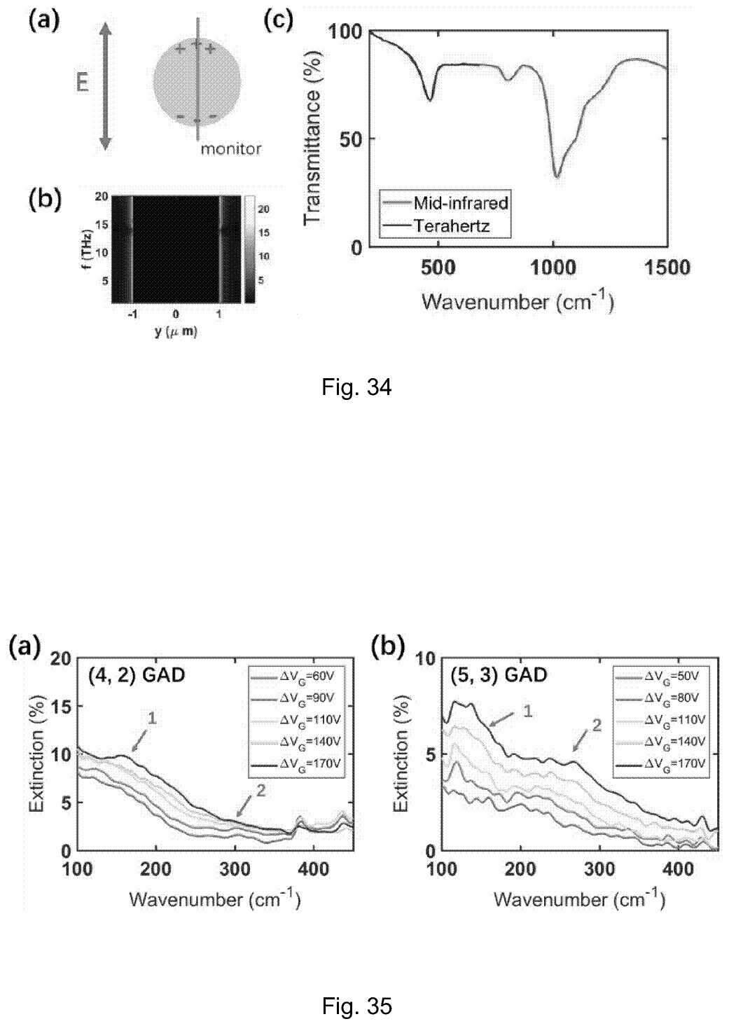

The gold disks in our hybrid devices, which have almost the same size as the graphene antidots, have resonances at much higher frequencies than the GSP resonances of the graphene anti-dot array. Nevertheless, due to the lightning-rod effect, the gold disks produce highly enhanced field distributions near their edges in a broad frequency range. As shown in FIG. 34(a)-(b), we simulate the optical response and near field intensity distribution of a gold disk array (3, 2) on Si/SiO₂ wafer. A line monitor is put in the center of the gold disk to monitor the near field profile. Except for close to the optical phonon frequency of silicon dioxide (˜14 THz), the near-field intensity around the gold disk is enhanced by ˜20 times near its edge in a broad terahertz frequency range. We also fabricate gold disk arrays without any graphene structures, and the experimentally measured transmission of the gold disk array (3, 2) is shown in FIG. 34(c). Indeed, the resonant response of the gold disk array (˜1000 cm⁻¹) is far away from the frequency range of the graphene anti-dot GSP resonances.

### 3.4.3 Measured Extinction Spectra of Graphene Devices

The extinction spectrum of graphene antidot device (GAD) (4, 2) and (5, 3) applied with different back gate voltage are measured and shown in FIG. 35. Similar to the simulation results, two resonant modes can be observed in the measured transmission extinction spectra as indicated in the red arrow of FIG. 35. The discrepancies between the measured and simulated spectra are mainly due to fabrication variation, which leads to the sample geometries being different from the designs. As the applied voltage or graphene Fermi level increases, the transmitted extinction has a larger value, which contains the plasmon response of graphene and the resonant extinction response of graphene. The resonant extinction peaks are relative weak and they can only be clearly observed when the fermi level of graphene is high. With the increase of the graphene fermi level, two resonant peaks shift to higher frequency because of the increasing conductivity of graphene.

### 3.4.4 Measured Extinction Spectra of Hybrid Devices

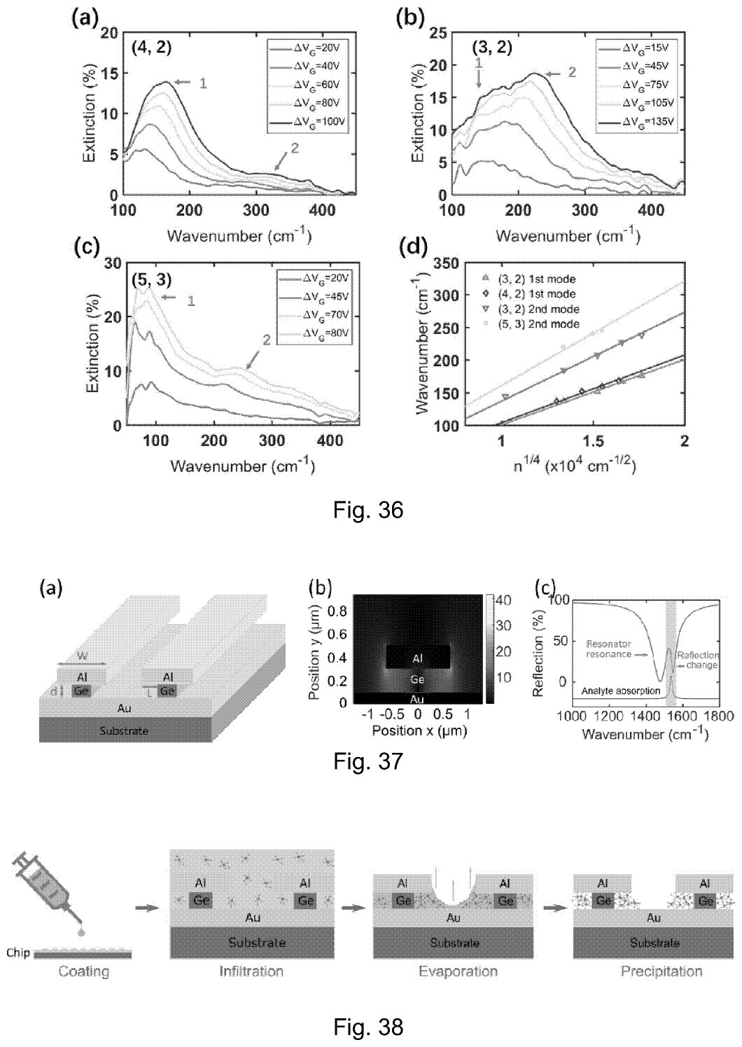

FIG. 36 shows the measured transmission extinction spectra of several fabricated devices of specified geometries. Different resonant modes can be observed in the measured transmission extinction spectra, which is consistent with the simulation results. The transmission extinction peaks of these hybrid devices are clearly a few times stronger than those of the corresponding graphene antidot devices. Although the main resonance peak of the hybrid device (5, 3) is difficult to accurately identify because of the limited frequency range of our measurement system, its transmission extinction reaches as high as 25%, which is ˜4 times that of the bare antidot (5, 3) device. It is also interesting to notice that, thanks to the enhancement induced by the gold disks, some higher-order GSP resonance modes of the hybrid devices are clearly observed (e.g., the second resonance mode of the (4, 2) and (5, 3) devices), whereas the corresponding modes are not as visible in the spectra of the bare graphene antidot structures. All of the observed GSP resonance peaks shift to higher frequency when the gate voltage is tuned from 15 to 135 V (corresponding to the graphene Fermi level varying from ˜0.14 to ˜0.41 eV). All of the extracted resonance frequencies follow the carrier-density-dependent frequency scaling law of GSPs

$$( ω ∝ {n}^{\frac{1}{4}} ∝ \sqrt{{E}_{F}} ) ,$$

as shown by the fitting results in FIG. 3.13(d).

### 3.5 Conclusion

In summary, we demonstrate a type of hybrid graphene-metal metasurface employing graphene antidots enclosing self-aligned gold disk cores. The gold disks function as THz optical antennas that effectively enhance the interaction between incident light and the tunable GSP resonances of the graphene antidots. Compared to the bare graphene antidot structures, the hybrid structures show ˜3 times stronger optical response (e.g., transmission extinction) and about an order of magnitude higher near-field intensity at the GSP resonances. These simple hybrid structure designs allow us to employ a self-aligned photolithography fabrication process that reliably achieves ˜100 nm critical feature size across large device areas. This hybrid graphene-metal metasurface design strategy and the convenient self-aligned fabrication process can be applied to other types of graphene-based plasmonic structures to further enhance various light-matter interactions for a broad range of applications, such as photodetectors, sensing, and nonlinear optics.

### 4. High-Sensitivity Nanophotonic Sensors with Passive Trapping of Analyte Molecules in Hot Spots

### 4.1 Introduction

Surface enhanced infrared absorption (SEIRA) spectroscopy has been attracting great interest since it was discovered in the 1980s, as it is able to deal with the weakness of the absorption cross sections of the infrared absorption. Given the fact that the amount of absorption by molecules is proportional to the intensity of the field experienced by them, the strong optical near-fields of nanophotonic structures are significant in enhancing the amount of absorption by molecules. A variety of photonic structures have been employed for SEIRA sensing, such as nano-rods, split-ring resonators, colloidal nano-particles, and metal-insulator-metal type structures. To enhance sensitivity, structures with nanometric gaps for achieving ultrahigh field confinement and enhancement were demonstrated. For example, sensors based on individual bowtie antennas with a sub-3 nm gap were able to resolve vibrational signals from a few hundred molecules in the gaps. Regardless of specific photonic structure designs, regions of highly enhanced near-field intensity, also referred to as hot spots, only occupy a small fraction of the total surface area of a photonic structure. Surface-enhanced molecular absorption is only significant in these hot spots. In general, higher field enhancement is typically associated with smaller hot spots.

To conduct SEIRA sensing, the analyte is usually coated over the entire sensor surface using methods such as the self-assembly of molecular layers, spin coating, DNA/protein immobilization, or the physical adsorption of biomolecules. A common drawback of these convenient methods is that analyte molecules are distributed across the entire sensor surface. Therefore, only a small percentage of all analyte molecules are delivered to the sensor hot spots that produce an enhanced molecular vibrational absorption signal, whereas the majority of the analyte molecules are outside of the hot spots and do not contribute significantly to the overall sensing signal. This issue can be a major limiting factor for the sensitivity performance of SEIRA sensors as well as optical sensors in general but has not been adequately addressed in most sensor designs. Enhancing the near-field intensity is an important design strategy that has been constantly improved by employing structures with ever-smaller hot spots; however, this approach does not necessarily lead to overall sensitivity improvement alone, as reducing the sizes of hot spots typically results in a smaller amount of analyte molecules delivered to the hot spots. Therefore, developing effective approaches for targeted and efficient delivery of analyte molecules to hot spots is perhaps an equally important aspect for optimizing optical sensor performance. One of the explored approaches is to build micro- and nano-fluidic structures, such as nano-pores or nano-gaps, to guide analyte solutions to hot spots. Although, in principle, this approach can ensure that all analyte molecules pass through the hot spots, as the molecules are still distributed in the solution and there is no concentrating mechanism, at any moment, the number of molecules in the hot spots is determined by the solution concentration. Active trapping mechanisms such as dielectrophoresis, optical trapping, and micro-bubble trapping are promising ways to concentrate and deliver nano-objects and large biomolecules (e.g., proteins and DNA). However, these trapping mechanisms require external energy sources such as a laser or an applied voltage and are not suitable for relatively small molecules. Super-hydrophobic artificial surfaces comprising arrays of micro-pillars have been employed for passively confining large biomolecules in diluted solutions to nano-photonic structures, which led to impressive sensitivity performance. However, this approach has not achieved targeted delivery of analyte molecules only to the hot spots of nanophotonic structures.

In this section, we present a nanophotonic sensor design that employs hot spot structures that can passively retain and concentrate an analyte solution as it evaporates and eventually traps precipitated analyte molecules inside the hot spots, significantly enhancing its SEIRA sensitivity. We demonstrated this passive trapping functionality using several molecular species (L-proline, D-glucose, and sodium chloride) as well as nano-particles (liposomes). These SEIRA sensors reliably produce clear spectral responses to the molecular vibrational absorption associated with picogram-level analyte precipitates. This section is a slightly modified version of published in Light: Science & Applications.

### 4.2 Design and Fabrication of Sensors

### 4.2.1 Design Rationale of the Sensor Device

The schematic of an example device structure is shown in FIG. 37(a). The designed optical resonators have a metal-insulator-metal type structure, which comprises a periodic array of Al ribbons on top of Ge ribbons, with an Au back reflector underneath. Ge was chosen mainly because it has relatively low optical loss in the target spectral region and can be deposited on metal films using the electron beam evaporator in our cleanroom facility, whereas in principle it can be re-placed by any other material with low optical loss such as Si. The Ge ribbons are narrower than the Al ribbons by hundreds of nm and therefore nano-trenches are formed on both sides of each Ge ribbon. Such a structure supports a resonance mode which can be excited by incident light polarized perpendicular to the Al ribbons, and the resonance frequency can be designed to target specific absorption lines of a molecular species by tailoring the geometrical parameters such as the Al ribbon width w, the nano-trench width L, and the Ge ribbon height d. FIG. 37(b) shows the simulated distribution of electric near field enhancement of an exemplary design at its resonance frequency. The electric field is mostly confined inside the nano-trenches and the highest electric field enhancement can reach over 40, which corresponds to more than 3 orders of magnitude intensity enhancement. Therefore, these nano-trenches are the hot-spots of our resonator design. Molecules located inside the nano-trenches experience high field intensity enhancement, which in turn leads to enhanced spectral response to the molecular vibrational absorption. As an example, FIG. 37(c) shows the reflection spectrum of a resonator design with its nano-trenches filled with an “imaginary” molecular species which has a single absorption line (black curve) near the resonance frequency of the resonator. The reflection spectrum clearly exhibits a Fano-resonance type feature near the molecular absorption line, as a result of the molecular vibrational mode interfering constructively or destructively with the enhanced electric field in the nano-trenches.

In addition to achieving a large field enhancement, another key advantage of incorporating these nano-trenches in a resonator design is that molecules in a low-concentration analyte solution can be passively trapped inside the nano-trenches as the analyte solution gradually evaporates. FIG. 38 illustrates such a passive molecule trapping process. A droplet of analyte solution can be introduced to the device surface using a micro-pipette. The analyte solution covers the entire resonator array as well as infiltrates into the nano-trenches. Subsequently, the solvent of the analyte solution gradually evaporates and the solution top surface gradually lowers, while the concentration of the solution increases. When the solution top surface is below the Al ribbon top surface, a concave profile of the solution surface forms between neighboring Al ribbons due to surface tension and the edge of solution is pinned at the side of the Al ribbons. As the solvent evaporates further, the concave part ruptures near its center and the solution is retained inside the nano-trenches and further concentrates, and eventually most of the analyte molecules precipitate inside and near the edges of the nano-trenches as the solvent completely evaporates.

### 4.2.2 Device Fabrication

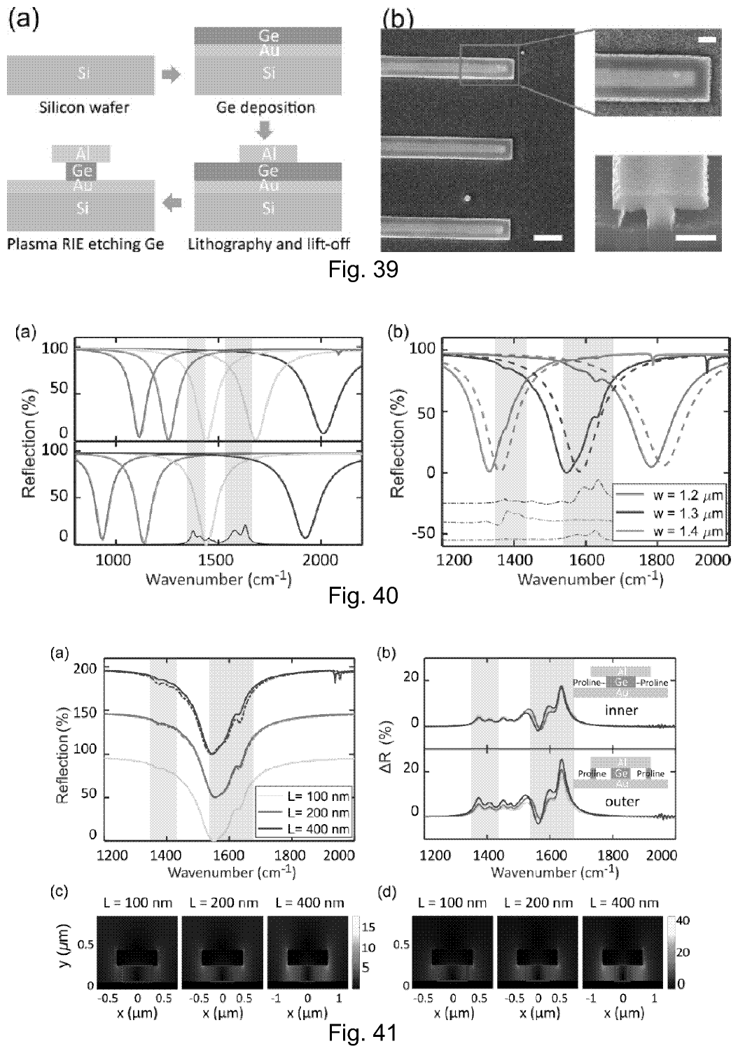

FIG. 39(a) illustrates the fabrication process to realize the designed device structures. Compared with nanophotonic sensors employing nanometric gaps or super-hydrophobic artificial surfaces, our device designs are easier to fabricate. The entire fabrication process involves only one lithography step and one dry etching step, which is similar to typical metal-insulator-metal photonic structures without nano-trenches. Each fabricated resonator array occupies an area of 300 μm by 300 μm. A silicon substrate was cleaned with acetone/isopropyl alcohol (IPA) in ultrasonic bath, followed by oxygen plasma cleaning. A 10 nm/200 nm thick Ti/Au film and a 200 nm thick Ge film were sequentially deposited on the silicon wafer using electron beam evaporation. The samples were spin coated with a ˜250 nm thick layer of poly(methyl methacrylate) (PMMA, molecular weight 495K, MicroChem, 495PMMA A4), and then a second layer of PMMA (molecular weight 950K, MicroChem 950PMMA A4). The samples were baked at 180° C. for 2 minutes. The ribbon arrays were patterned using electron beam lithography at 100 kV accelerating voltage, 10 nA beam current, and 1200 μC/cm² exposure dose. The exposed PMMA was developed in a 1:3 solution mixture of methyl isobutyl ketone (MIBK) and IPA at room temperature for 60 seconds. Then 5 nm Ti and 200 nm Al films were deposited with electron beam evaporation followed by a metal lift-off process in acetone, which formed the Al ribbon arrays. An oxygen plasma cleaning step was conducted to fully remove the PMMA residual or other organic contaminations. Using the Al ribbons as hard masks, the Ge layer was partially etched by reactive ion etching (RIE) with CF4 gas plasma (flow rate 50 sccm, pressure 50 mTorr, power 100 W). The RIE etching process not only removed the Ge film between Al ribbons, but also produced undercuts beneath the Al ribbons which formed the designed nano-trenches. After each 20 seconds RIE etching, SEM images of the samples were taken to determine whether the desired nano-trench length was achieved.

FIG. 39(b) shows scanning electron microscopy (SEM) images of the fabricated devices. In the top-view images of the structure (the left and upper-right panels), the bright edges of individual Al ribbons can be clearly seen. Adjacent to the bright Al ribbon edges are stripes exhibiting a dark gray color, which is in stark contrast to the bright color of the middle region of the Al ribbons. The dark stripes correspond to the nano-trenches, whereas the bright stripes in the middle correspond to the Ge ribbons underneath the Al ribbons. We also used a focused ion beam (FIB) to cut individual resonators and expose their cross-sections for SEM imaging. A cross-sectional SEM image of a cut resonator in the lower right panel of FIG. 39(b) clearly shows that the designed nano-trench structures were successfully formed using the developed process.

### 4.3 Simulation Results

Numerical simulations were conducted by using the finite difference time domain (FDTD) method to investigate how the Al ribbon width (w) and the nano-trench width (L) affect the optical response as well as sensing performance. FIG. 40(a) shows the reflection spectra of several resonator designs with different geometrical parameters. The top panel corresponds to designs with different w values (ranging from 800 nm to 1.4 μm) and a fixed L=200 nm, whereas the bottom panel corresponds to designs with different L values (ranging from 0 to 300 nm) and a fixed w=1 μm. The height of the Ge ribbon is fixed at 200 nm in these designs. These design parameters can be reliably realized using standard nanofabrication processes, and the corresponding wide spectral tuning range covers infrared vibrational absorption bands of a broad variety of molecular species. Furthermore, we also avoided any design parameter which may weaken the structural robustness of the devices, so that the devices can withstand the processes for introducing and removing the target analyte and be used for repeated measurements. As an example for demonstrating the sensing capability of these resonator designs, we choose L-proline, one of the amino acids, as the analyte molecule which has several vibrational absorption lines in the spectral range between 1400 cm⁻¹ and 1700 cm⁻¹ (see the black solid line and shaded regions in FIG. 40(a)). FIG. 40(b) shows the simulated reflection spectra of three resonator designs with the specified Al ribbon widths and a fixed nano-trench width/=400 nm before (dashed curves) and after (solid curves) placing 100 nm wide proline at the inner side of the nano-trenches (i.e., adjacent to the Ge ribbon sidewall). The resonance frequency of the resonators is tuned across several proline absorption lines in this spectral range. The extracted reflection changes due to the added proline are plotted as the dot-dashed curves. As expected, the spectral response to a specific proline absorption line is the strongest when the resonance frequency of a resonator matches that absorption line.

We further investigate the influence of the nano-trench width on the sensing performance in FIG. 41(a), which shows three resonator designs with different L values (as well as different w values so that their resonance frequencies are similar), with 100 nm wide proline filling either the inner side (solid curves) or the outer side (dashed curves) of the nano-trenches. Note that for L=100 nm, the 100 nm wide proline simply fills the entire nano-trenches. The extracted spectral changes due to the added proline in the nano-trenches are plotted in FIG. 41(b). Comparing the different spectra in FIGS. 41(a) and (b), we find that the resonator designs with wider nano-trenches produce a stronger spectral response, and the spectral response is larger when the same amount of proline is located at the outer side of the nano-trenches than at the inner side. These observations can be explained by the electric field distributions in these nano-trenches. The electric near field distributions of the three resonator designs at the frequencies 1452 cm⁻¹ and 1630 cm⁻¹ are shown in FIG. 41(c) and FIG. 41(d), respectively. It can be clearly seen that overall the designs with wider nano-trenches have higher field enhancement, and the field enhancement in a nano-trench is always larger at its outer side than at its inner side.

### 4.4 Experiments Results

### 4.4.1 Trapping Functionality for Small Molecules

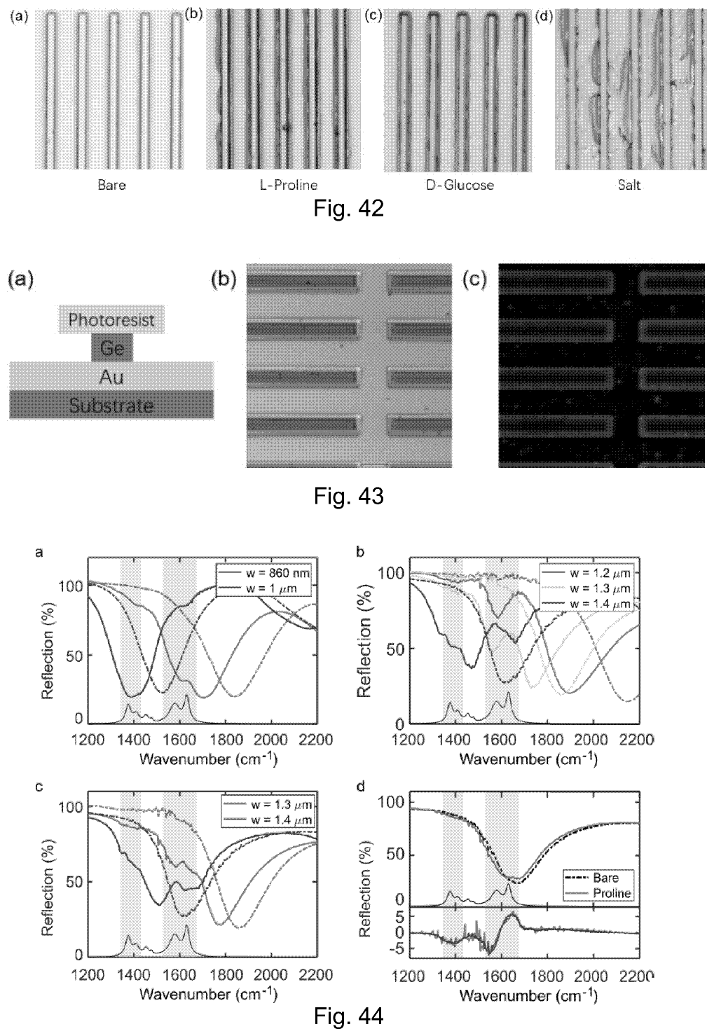

After device fabrication, we tested the proposed passive molecule trapping functionality of the nano-trench structures. Three water-based solutions of different chemicals were prepared: L-proline (1 mg/mL), D-glucose (1 mg/mL), and regular table salt (mostly sodium chloride, 40 μg/mL). A small droplet (˜1 μL) of each solution was dropped onto the device surface using a micro-pipette, which dried slowly under ambient conditions and eventually led to the precipitation of the dissolved chemical on the device surface. The concentrations of these solutions are high enough that the precipitated chemicals should completely fill most of the nano-trenches, which can be easily observed. Top-view optical microscopy images of the device structures with precipitated chemicals are shown in FIG. 42. Compared with the bare device image in FIG. 42(a), most of the precipitated chemicals accumulated near the edges of the Al ribbons, whereas the regions between adjacent Al ribbons were mostly clean. Although the optical images did not provide direct observation of chemicals inside the nano-trenches, they agreed with our expectation that the precipitation of dissolved molecules in an evaporating solution takes place mostly inside and near the nano-trenches. A video recording of the process of solution drying on the device surface (see video from ref) also clearly shows that as the solution evaporated, it concentrated inside and near the nano-trenches, which eventually evaporated completely and led to precipitation of the dissolved chemical.

### 4.4.2 Trapping Functionality for Small Molecules

In addition to trapping molecules in a solution, we found that such nano-trench structures can also effectively trap nano-particles in a gradually dried nano-particle suspension. We used a suspension of liposomes containing Cyanine 5 (Cy5) fluorescent dyes in our experimental demonstration. The Cy5-labeled liposomes have a diameter of ˜100 nm. The fluorescent dyes allowed us to observe the spatial distribution of liposomes using a confocal microscope.

### 4.4.2.1 Preparation of Liposomes Containing Fluorescent Dye Molecules

The Cy5-labelled liposomes were prepared using the ethanol injection method. Briefly, a lipid mixture of 1,2-di-O-octadecenyl-3-trimethylammonium propane (DOTMA, Avanti Polar Lipids, 890898P), cholesterol (Sigma-Aldrich, C3405) and D-α-Tocopherol polyethylene glycol 1000 succinate (TPGS, Sigma-Aldrich, 57668-5G) was prepared at DOTMA: cholesterol:TPGS=49.5:49.5:1 molar ratio in ethanol. Empty liposomes were prepared by injecting 1 part of the lipid mixture to 9 parts of 20 mM HEPES buffer. Then, Cy5 labelled oligonucleotides (Cy5-ODN, 5′-Cy5-GGCTAAATCGCTCCACCAAG-3′, Alpha DNA) were mixed with empty liposomes at the Cy5-ODN to lipid mass ratio of 1:10. The mixture of empty liposomes and Cy5-ODN was sonicated at room temperature for 10 minutes to form liposomes containing Cy5-ODN.

### 4.4.2.2 Fluorescence Imaging of Liposomes Containing Fluorescent Dye Molecules

The device structures used for trapping liposomes are similar to our SEIRA sensors, except that the top Al ribbons were replace by photoresist (PR) ribbons for direct imaging of fluorescence emitted from the nano-trenches (see FIG. 43(a)). The liposome solution at Cy5-ODN concentration of 1 mg/mL was delivered to the device surface with the dip-coating method. The device was then left in ambient condition to dry. We used a Zeiss LSM 710 Confocal Microscope equipped with a Zeiss In Tune laser (emission wavelength can be tuned in the range of 488 nm to 640 nm) and spectral detectors. As this is an inverted microscope system, the device was placed with the top surface facing down onto a cover glass. The reflection image of the device structure (FIG. 43(b)) was taken with the excitation wavelength set to 488 nm and the detection wavelength range set to 506 nm, while the fluorescence image of the Cy5-labelled liposomes deposited on the device (FIG. 43(c)) was taken with the excitation wavelength set to 640 nm and the emission wavelength range set to 707 nm. As shown in FIG. 43(b)-(c), the intensity of fluorescence was much higher in the nano-trench regions than in the other regions, such as between the Al ribbons, which indicated that most of the liposomes were trapped in the nano-trenches.

### 4.4.3 Device Sensing Performance

### 4.4.3.1 Measured Spectrum for Device Coating with L-Proline

To evaluate the sensing performance of our devices, we first used L-proline as the target analyte molecule and characterized the spectral responses of our SEIRA sensors to trace amounts of L-proline precipitated from low-concentration solutions. For each measurement, a droplet of ˜1 μL of water-based proline solution with a certain concentration was dropped on the device surface and gradually dried under ambient conditions. Reflection spectra of the devices before and after introducing the analyte were measured with a Fourier transform infrared spectrometer (FTIR) connected to an infrared microscope. We started with a proline solution of 10 μg/mL (˜87 μM) and applied it to two sets of devices with either 200 nm wide nano-trenches or 450 nm wide nano-trenches. The measured reflection spectra are shown in FIG. 44(a)-(b). Compared with the reflection spectra of the bare devices (dashed lines), the reflection spectra of both sets of devices after proline was introduced show a significant peak frequency shift and clear spectral features associated with the proline absorption lines. As expected, such spectral features are stronger when the corresponding proline absorption lines are closer to the peak resonance frequency of the resonators. Overall, the set of devices with 450 nm wide nano-trenches produce considerably stronger spectral responses than those with 200 nm wide nano-trenches, which is consistent with our simulation results in FIG. 41. As proline has a relatively high solubility in water, the proline precipitate on the devices can be completely removed by rinsing with deionized water. This allowed us to conduct repeated proline sensing measurements using the same devices on proline solutions of various lower concentrations to explore the sensitivity limit of these devices. FIG. 44(c) shows the reflection spectra of two devices with 1.3 μm- and 1.4 μm-wide Al ribbons and 450 nm wide nano-trenches in response to ˜1 μL proline solution at a concentration of 1 μg/mL (˜8.7 μM).

The reflection spectrum change owing to the 1 μg/mL proline solution still has a considerable peak frequency shift and relatively strong spectral features associated with the proline absorption lines. FIG. 44(d) upper panel shows the reflection spectrum change owing to ˜1 μL proline solution at an even lower concentration of 0.2 μg/mL, which is smaller but nevertheless clearly visible. Note that the total amount of proline in one 1 μL droplet of 0.2 μg/mL proline solution is only 200 μg, and only a small fraction of this total amount was precipitated within the device area (˜150 μm by 150 μm), from which the reflection spectra were measured. To better visualize the spectral response to such a trace amount of analyte molecules, we extracted the difference between the reflection spectrum of the device with the proline precipitate and that of the bare device, which is plotted in the lower panel of FIG. 44(d). As these devices have zero transmission, the reflection change is essentially the negative of the absorption change. To extract the differential spectrum only due to the analyte absorption lines, the reflection spectrum of the bare device was first redshifted so that the broad resonance in the two spectra overlapped across almost the entire spectral range, except around the proline absorption lines (see Section C). This differential spectrum (the red curve in FIG. 44(d) lower panel) shows a clear feature with an asymmetric line shape near the two proline absorption lines at 1630 cm⁻¹ and 1577 cm⁻¹ and a peak-to-peak amplitude of ˜10%, which is significantly above the noise level of the FTIR measurement (1%). The observed line shape is also in good agreement with the simulated results in FIG. 40 and FIG. 41, although the two proline absorption lines were not individually resolved as in the simulation. Furthermore, another weaker (˜3% amplitude) spectral feature associated with the proline absorption lines near 1450 cm⁻¹ was also observed. The measurements were conducted under ambient conditions, and therefore, the spectra contain interference from water vapor absorption in the optical path within the spectral region of interest, which corresponds to the narrow peaks in the lower panel of FIG. 44(d). However, as these water vapor absorption lines are narrow and at fixed wavelengths, they can be removed from (or significantly reduced in) the measured spectra with appropriate data processing. The blue curve in the lower panel of FIG. 44(d) is the differential reflection spectrum after performing a data processing procedure for removing the water vapor absorption lines. The spectral features associated with the proline absorption lines are much higher than the spectral noise and a few times larger than any residual water vapor absorption lines. According to the International Union of Pure and Applied Chemistry (IUPAC) definition of the limit of detection (LoD), the obtained spectral signal should be well above the LoD of these sensors using our measurement setup (see section 4.4.3.3). On the other hand, the LoD of our sensors can also be improved by optimizing our experimental setup. For example, the water vapor absorption lines can be removed or greatly reduced by purging the measurement system with nitrogen gas, and the open-path part of the experimental setup can be enclosed by opaque shields to reduce background radiation fluctuation.

### 4.4.3.2 Measured Spectrum for Device Coating with d-Glucose

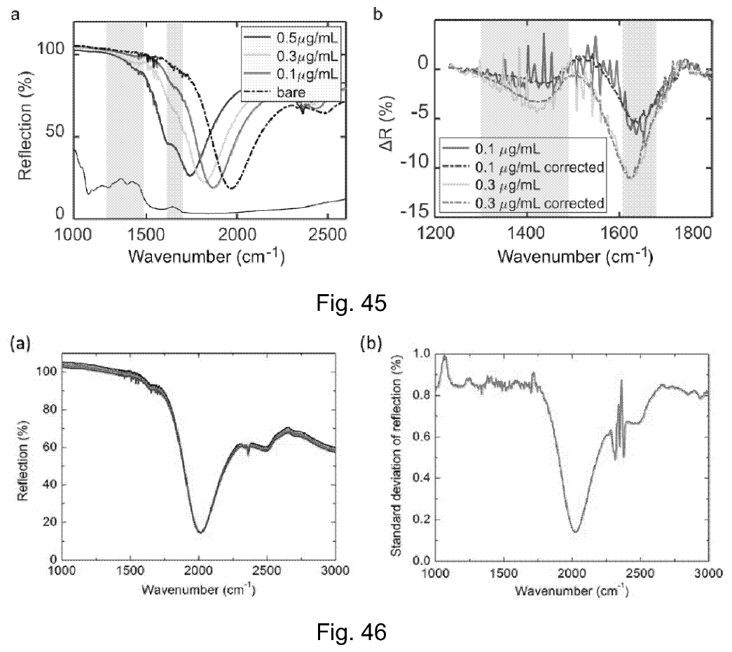

As another example to demonstrate the sensing performance and versatility of our devices, we further conducted sensing of D-glucose in low-concentration solutions. Glucose has an important role in human metabolism, and monitoring the glucose concentration in blood is crucial for people with diabetes. Glucose has multiple absorption lines in the 1000 cm⁻¹ to 1500 cm⁻¹ spectral range, with several of them forming a relatively broad absorption band from 1330 cm⁻¹ to 1460 cm⁻¹ (see FIG. 45(a)). There is also a relatively weaker absorption line at ˜1640 cm⁻¹, which is due to adsorbed water, as glucose is hygroscopic. These glucose molecular absorption lines are within the spectral range of our SEIRA sensors developed for sensing proline. We prepared glucose solutions of various concentrations and followed the same experimental procedure to deliver droplets of the solutions to the device surface and characterized the spectral change due to the glucose precipitate. FIG. 45(a) shows the spectral response of a device to a 1 μL droplet of glucose solutions at three different concentrations, i.e., 0.5 μg/mL (˜2.8 μM), 0.3 μg/mL (˜1.7 μM), and 0.1 μg/mL (˜0.56 μM). The device produced a strong response to all three glucose concentrations. The spectral feature associated with the 1640 cm⁻¹ absorption line was considerably stronger than that associated with the 1400 cm⁻¹ absorption band, mainly because the resonance frequency of the device was closer to the 1640 cm⁻¹ absorption line. The differential spectra extracted from the measurement results in FIG. 45(a) (both before and after applying the correction for the water vapor absorption lines) are shown in FIG. 45(b). The spectral feature associated with the 1640 cm⁻¹ absorption line reached an amplitude of ˜10% at a 0.3 μg/mL concentration and ˜5% at a 0.1 μg/mL concentration, whereas the 1400 cm⁻¹ absorption band also led to a spectral feature with a different amplitude.

### 4.4.3.3 Limit of Detection

According to the definition of LoD by IUPAC, the LoD expressed as a concentration c_{L} is derived from the smallest signal x_{L} that can be detected with reasonable certainty. To ensure a relatively high certainty, the convention is to set the LoD threshold signal as x_{L}=x̅_{B}+3s_{B}, where x̅_{B} is the mean value of repeated blank measurements (i.e., without analyte), and s_{B} is the standard deviation of such blank measurements. In order to characterize the standard deviation of the blank measurements (i.e., reflection spectra of bare devices) using our measurement setup and parameter settings, we conducted 10 independent measurements of the reflection spectrum of a bare device sequentially, which are shown in FIG. 46(a). The standard deviation of these spectral measurements was then calculated at each frequency point, which is plotted in FIG. 46(b). Clearly, in the entire spectral range from 1000 cm⁻¹ to 3000 cm⁻¹, the standard deviation remains below 1%. Therefore, in our experiments, any spectral change around the absorption lines of an introduced analyte with more than 3% peak difference can be considered with a high confidence level as being above the LoD threshold signal. The fact that the standard deviation spectrum in FIG. 46(b) has a similar shape as the reflection spectrum of the device in FIG. 46(a) suggests that the dominant noise source may be the fluctuation of background radiation incident on the device. Therefore, by enclosing the entire optical path of our measurement setup with radiation shields (which is not the case currently) may further reduce the blank measurement fluctuation and improve the LoD.

### 4.4.3.4 Reliability of Our Devices

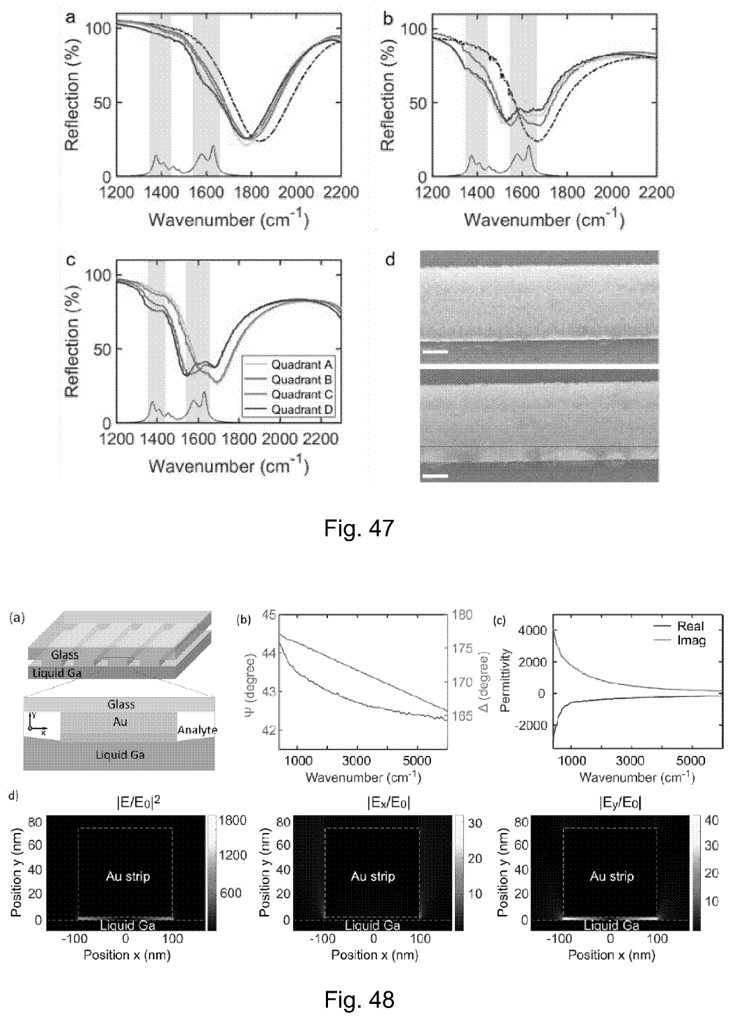

To test the reliability of our devices for sensing such low-concentration analyte solutions, we chose several devices to repeat the same experimental process multiple times for each proline solution concentration. After each measurement, the device was thoroughly cleaned with deionized water to completely remove the proline precipitate, which was confirmed by measuring the reflection spectrum of the cleaned device before we applied a new droplet of solution for the next measurement. The reflection spectra of repeated sensing experiments for a 1 μg/mL proline solution using a device with 200 nm wide nano-trenches and another device with 450 nm wide nano-trenches are presented in FIG. 47(a) and FIG. 47(b), respectively. Both devices produced an observable reflection spectrum change in each measurement. However, the amplitude of the spectral change varied from measurement to measurement. This spectral response variation across repetitions of a nominally identical experimental process is mainly owing to the distribution variation of precipitated proline across the device area, which is non-uniform and different each time. Comparing the spectral changes in FIG. 47(a) with those in FIG. 47(b), we can see that the device with 450 nm wide nano-trenches consistently produced a significantly larger spectral response than the device with 200 nm wide nano-trenches and hence can more reliably sense trace amounts of proline precipitate despite the experimental variation. FIG. 47(c) shows the reflection spectra measured from the four quadrants of the device with 450 nm wide nano-trenches (the total device area is 300 μm by 300 μm, and each quadrant is 150 μm by 150 μm) after two droplets of 0.2 μg/mL proline solution were introduced to the device surface (two droplets were used for this measurement to enhance the response signal). The spectra in quadrants C and D clearly exhibit a stronger response than those of quadrants A and B. This is again a result of the non-uniform distribution of the precipitated proline across the device surface, which can be seen from the SEM images in FIG. 47(d) taken from a view angle 45° off the vertical direction. The top and bottom SEM images in FIG. 47(d) represent the typical distribution of precipitated proline in quadrant B and quadrant D of the device in this particular experiment, respectively. These SEM images also clearly show that the proline precipitate was indeed in the nano-trenches, but it only occupied a small fraction of the nano-trench volume when the analyte solution was of such a low concentration.

### 4.5. Discussion and Conclusion

In terms of the total amount of analyte molecules contained in a solution droplet, our most sensitive devices reliably produced clear spectral responses to 200 μg of proline and 100 μg of glucose. However, as a 1 μL solution droplet typically covers a circular area with a diameter of ˜2 mm on a device chip, whereas each sensor (resonator array) only occupies a square area of 300 μm by 300 μm, the actual amount of analyte molecules precipitated within the sensor area should be more than an order of magnitude smaller than the total amount contained in the solution droplet. Moreover, as the reflection spectra were measured from a 150 μm by 150 μm area within each resonator array, the number of analyte molecules involved in a spectrum measurement should be on average approximately a quarter of the amount within the entire sensor area. If we assume that on the macroscopic scale, the analyte molecules are distributed uniformly across the entire droplet area, then we can estimate that the spectral responses shown in FIG. 44(d) and FIG. 45(b) are owing to only ˜1.4 pg of proline and ˜0.7 pg of glucose, respectively. To put such sensitivity levels in another perspective, if these trace amounts of analyte molecules form a uniform thin film covering the measurement area, they would correspond to an ˜0.5 Å thick proline film and an ˜0.2 Å thick glucose film, respectively. Such thickness values are significantly less than those of the corresponding molecular monolayers. Therefore, the sensitivity of our current devices represents state-of-the-art SEIRA sensing, although the field enhancement in the hot spots of these nanophotonic resonators is orders of magnitude lower than that of some previously demonstrated sensor structures employing nanometric gaps. The passive molecule trapping functionality of the nano-trench structures in our devices plays a crucial role in achieving such high sensitivity performance. By improving the measurement setup (such as using a purged environment to reduce water absorption) or adding a simple structure on the device surface to confine the droplet within the resonator array area, our current devices should be able to produce clear SEIRA responses to even lower solution concentrations. On the other hand, the current nanophotonic resonator designs can be optimized to achieve much larger field enhancement while preserving the molecule trapping functionality, which may lead to further improvement of the device sensitivity by orders of magnitude.

In summary, we experimentally demonstrated a proof-of-concept design of nanophotonic SEIRA sensors employing nanoscale structures that not only form photonic hot spots with large-field enhancement but also have the functionality of concentrating and trapping analyte molecules in an evaporating solution in hot spots, hence leading to significantly improved SEIRA sensing performance. The trapping mechanism requires no external energy source (i.e., passive) and is a result of the evaporation process of liquid in contact with the designed nano-structures and therefore is not limited to specific molecular species. We experimentally demonstrated that the trapping functionality of our devices applies to various molecular species and nano-particles, such as liposomes. To investigate the sensing performance of our SEIRA sensors, we used L-proline and D-glucose as target analyte molecules and achieved reliable sensing of precipitated analyte molecules with a mass down to ˜1 pg, which corresponds to significantly less than one monolayer of analyte molecules when averaged over the entire measurement area. The sensitivity may be further improved by orders of magnitude with both improvements of the experimental setup and optimization of the device structure. The demonstrated SEIRA sensor design strategy can also be applied to other types of optical sensors. In addition to molecular sensing applications, such device structures can also be used for sensing and/or manipulating nanoscale objects, including exosomes, viruses, and quantum dots.

### 5. Liquid-Metal-Based Nanophotonic Structures for High-Performance Seira Sensing

### 5.1 Introduction

Here, we show that employing liquid metals, such as liquid gallium, is a convenient and effective approach to realizing high-performance SEIRA sensors with nanometric gaps filled with analyte molecules. As a proof-of-concept demonstration, we developed a SEIRA sensor structure comprising an array of metallic nanostrips separated by a nanometric dielectric layer from a metallic ground plane, which were essentially an array of nano-patch antennas. The nanometric dielectric layer was the sensing target, i.e., a thin film of analyte molecules, which was coated on the metallic nanostrips via physical/chemical adsorption or spin-coating. Subsequently, liquid gallium was added to cover the thin analyte film and function as the ground plane of the nano-patch antennas. Thanks to the highly confined and enhanced electric field in the nanometric gaps between the metallic nanostrips and the liquid gallium, the molecular vibrational signals associated with the analyte film can be substantially enhanced. These SEIRA sensors exhibited state-of-the-art sensing performance for nanometric analyte thin films, such as monolayer 1-octadecanethiol (ODT). Our experimental results also indicate that the liquid gallium did not cause damage to or change the properties of the analyte films. In addition, the liquid gallium can be conveniently removed completely from the sensor surface after a measurement, which makes it easy to reuse these sensors. This work demonstrates several key advantages of using liquid gallium for SEIRA sensing applications, and also points toward other potential applications of liquid metals in a variety of photonic structures and devices operating in the infrared spectral region.

### 5.2 Sensor Design and Fabrication

### 5.2.1 Design Rational of the Liquid Gallium Based Sensor

FIG. 48 shows the schematic of our SEIRA sensor design employing a nano-patch antenna structure. The thin analyte film is sandwiched between patterned gold nanostrips and a metallic ground plane. Rather than using a conventional noble metal such as gold or silver, liquid gallium is employed to form the ground plane, which has several key advantages. First of all, the fluidic nature of liquid gallium allows it to be straightforwardly spread on the analyte film and form the nano-patch antenna structures, without the need for any bulky equipment such as a metal evaporator. Such a sensor design makes the delivery of analytes into the nanometric sensing hot spots (i.e., the nanometric gaps) convenient and efficient, as the liquid gallium ground plane is added after the analyte thin film has been introduced onto the gold nanostrips. Secondly, gallium has a low melting point (˜29.7° C.) close to room temperature and hence is convenient to use. In addition, gallium is non-toxic and biocompatible, which are particularly important properties for a variety of biosensing applications. Furthermore, liquid gallium has favorable metal properties for photonics applications, including a relatively high bulk plasma frequency (around 15 eV in terms of energy) and low extrinsic loss thanks to the inherently smooth liquid surface and the absence of grain boundaries. Taking advantage of such material properties, liquid gallium has previously been exploited for plasmonics applications in the visible spectral region as well as for terahertz photonics. Nevertheless, potential applications of liquid gallium for mid-infrared photonics have largely not been explored.

We measured the permittivity function of liquid gallium across a wide infrared spectral region using spectroscopic ellipsometry, and the results are shown in FIG. 48(b)-(c). The large absolute values of its permittivity function indicate that liquid gallium behaves as a good metal in this spectral region. Based on the experimental permittivity data, we performed full-wave simulation of the designed nano-patch antenna structures with a liquid gallium ground plane. FIG. 48(d) shows that such nanophotonic structures can indeed achieve ultra-high confinement and enhancement of electric field intensity in the nanometric gaps (by about 3 orders of magnitude), which in turn can lead to large enhancement of SEIRA signals associated with the molecular vibrational absorption from the analytes located in these gaps. In addition, such nano-patch antenna structures allow for nearly perfect spatial overlap between an analyte thin film coated on the gold nanostrips and the highly enhanced electric field. Therefore, the presented sensor designs are especially suitable for sensing analytes in the form of continuous nanometric thin films.

### 5.2.2 Nanofabrication Processes of Sensor Chips

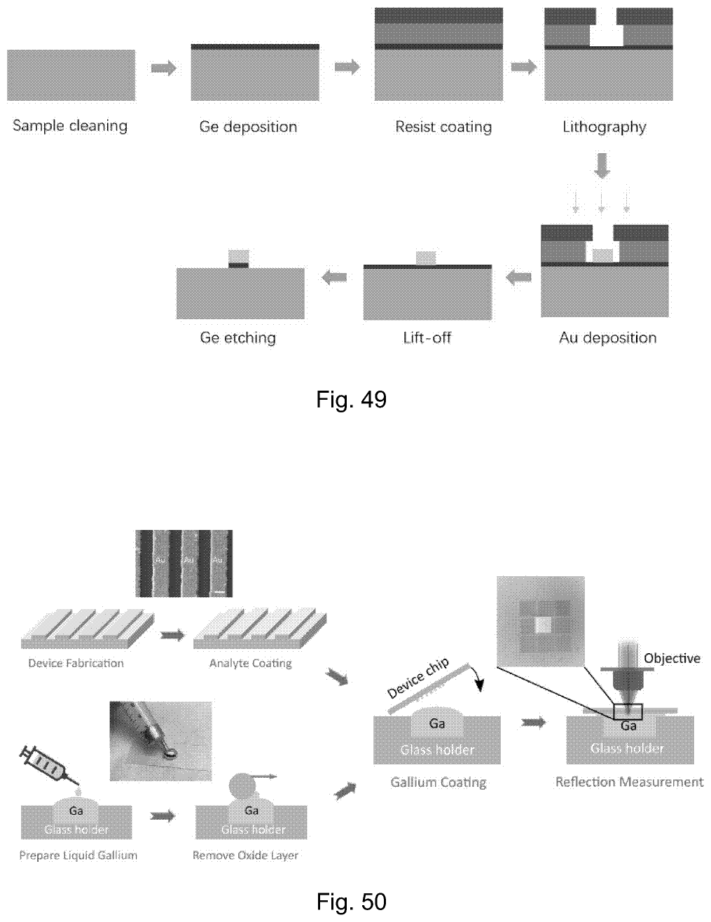

FIG. 49 shows the fabrication processes of the sensor devices. The substrates (glass coverslips or CaF2 substrates) were cleaned with acetone/isopropyl alcohol (IPA) in an ultrasonic bath, followed by oxygen plasma cleaning. A 10 nm thick Ge film was then deposited on the substrate using an electron-beam evaporator to provide a conductive layer, so that the charging effect can be mitigated during the subsequent electron-beam lithography step. A bi-layer electron-beam lithography resist having a ˜250 nm thick bottom layer of copolymer (MicroChem MMA (8.5) MAA EL 9) and a ˜70 nm thick top layer of PMMA (molecular weight 950K, MicroChem 950 PMMA A2) were spin-coated on the substrate surface, followed by baking on a hot plate at 180° C. for 2 minutes. Nanostrip arrays were patterned using electron-beam lithography at a 100 kV accelerating voltage, 2 nA beam current, and 800 μC/cm² exposure dose. The samples were immersed in a mixture of methyl isobutyl ketone and IPA (1:3) at room temperature for 45 seconds to develop the lithography patterns. Subsequently, 2 nm Ti and 70 nm gold were deposited on the sample surface using the electron-beam evaporator, followed by a metal lift-off process in acetone to form the gold nanostrip arrays.

### 5.2.3 Procedure for Preparing Liquid-Gallium-Based Sensors

FIG. 50 schematically shows the preparation procedure of our sensors. Sensor chips having arrays of gold nanostrips on a glass or calcium fluoride (CaF₂) substrate were fabricated as we described in the above section. Then the analyte molecules were coated on the surface of the gold nanostrips with methods such as physical/chemical adsorption or spin-coating, which are widely used methods for introducing analytes to nanophotonic sensors. Solid gallium in a syringe was melted by slightly warming up the syringe on a hot plate to above 30° C., and then a small amount of liquid gallium (about 0.1 mL) was injected into a glass holder. Although room temperature is slightly lower than the melting point of liquid gallium, it is not necessary to provide continuous heating because the liquid gallium can maintain its liquid phase even significantly below its melting point thanks to the super-cooling effect. Liquid gallium exposed to air has a thin oxide layer on the surface, which can be peeled off by simply scraping the liquid gallium surface with a thin plastic rod. After removing the oxide layer, the sensor chips coated with the analyte thin films were immediately placed on the liquid gallium to form the complete nano-patch antenna structures. A flat and mirror-like liquid gallium surface was usually observed, which indicated the formation of a good contact interface between the liquid gallium and the sensor chip. Subsequently, the infrared reflection spectrum of the complete nano-patch antenna structures was measured with the light incident from the transparent substrate side of the sensor. Compared with the conventional methods of depositing solid noble metals, such as evaporation or sputtering, this method of coating liquid metal to complete the sensor structures is simple and quick to operate and does not require expensive and bulky equipment, which significantly reduces the cost and is suitable for point-of-care sensing applications.

### 5.3 Experimental Results

### 5.3.1 Sensor Chips Preparing with Monolayer ODT

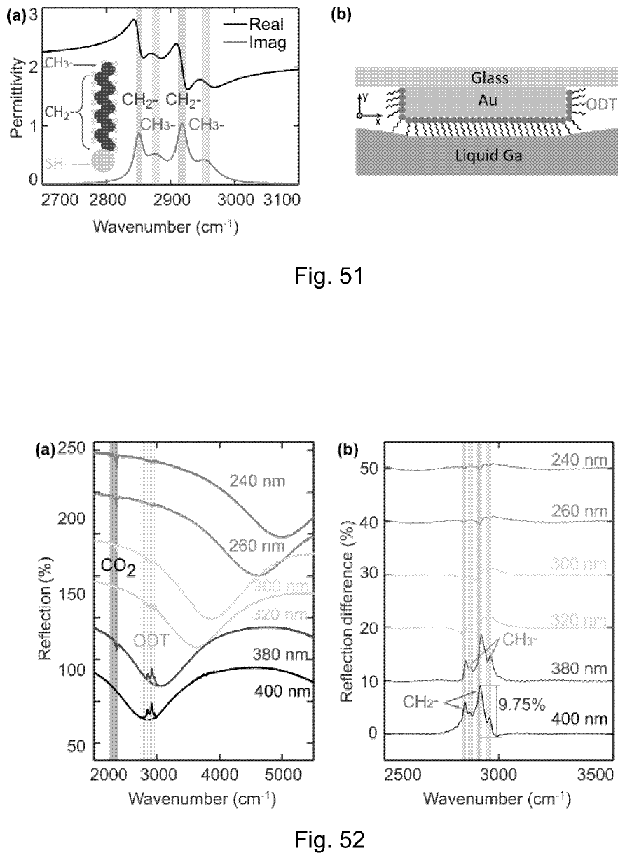

For a proof-of-concept demonstration of the sensor's performance, we firstly chose ODT as the model analyte. ODT is one of the alkanethiols which can form a self-assembled monolayer (SAM) with a thickness of ˜2.4 nm on gold surface. The permittivity of SAM ODT was extracted from experimental data (see Section A) and is shown in FIG. 51(a). ODT has four vibrational modes around 2900 cm⁻¹, which correspond to the symmetric and anti-symmetric stretching vibrations of CH₂- and CH₃-groups, respectively.

Multiple arrays of gold nanostrips with widths ranging from 200 nm to 400 nm were fabricated, so that the resonances of the corresponding nano-patch antennas can cover a wide spectral range that contains the ODT vibrational modes. The thickness of the gold nanostrips was 70 nm, and the duty cycle of all the gold nanostrip arrays was 50%. These design parameters were chosen to balance the optimization of the SEIRA sensing performance and the device fabrication reliability. To form the SAM ODT on the surface of the gold nanostrips, the sensor chips were immersed in 1 mM ethanol-based ODT solution which was prepared by adding 86 mg ODT into 300 mL ethanol. The fabricated sensor chips were first cleaned in hydrochloric acid for 2 mins to remove ionic and metallic contaminations on the gold surface, followed by rinsing in deionized water. Then the sensor chips were immersed in acetone and IPA to remove organic residuals. After an oxygen plasma cleaning to further remove organic residuals and modify the surface potential, the sensor chips were immediately immersed in the 1 mM ODT solution for at least 12 hours. Then the sensor chips were taken out of the ODT solution and rinsed in ethanol thoroughly to remove unbound ODT molecules. The ODT molecules form strong S—Au bonds on gold surfaces, leading to the formation of SAM ODT that completely covers the surface of the gold nanostrips and is not affected by the process of rinsing with ethanol. In contrast, the ODT molecules on the surface of the substrate or above the SAM ODT are unbound can be easily removed by the ethanol rinsing process. Subsequently, the sensor chips were placed on liquid gallium to form the complete sensor structures before the spectroscopic measurement, as shown by the schematic in FIG. 51(b). Our experimental observations indicate that this sample preparation procedure can reliably form SAM ODT that completely covers the gold surface. If the SAM ODT did not completely cover the gold surface, the liquid gallium would make direct contact with and erode the exposed regions of the gold surface, which was rarely observed in our experiments. The fact that the formation of SAM ODT on gold surface is a relatively reliable process suggests that the performances of our sensors and the different SEIRA sensors reported in the literature for sensing SAM ODT can be directly compared.

### 5.3.2 Measured Spectrum for Device Coating with SAM ODT

The measured reflection spectra of the sensors with varied gold nanostrip widths are shown in FIG. 52(a). The incident light was linearly polarized perpendicular to the gold nanostrips. The different sensors exhibited resonances (i.e., the reflection dips in FIG. 52(a)) covering a wide spectral range, from ˜2800 cm⁻¹ to ˜4900 cm⁻¹, as the width of the gold nanostrips varied from 400 nm to 240 nm. As highlighted by the gray region in FIG. 52(a), the molecular vibrational modes of the SAM ODT near 2900 cm⁻¹ coupled to the resonances of the sensors, which resulted in significant modulations of the reflection spectra in this spectral region. In addition, such molecular vibrational signals (i.e., the modulation of the sensors' reflection spectra) associated with the SAM ODT became much stronger when the resonant frequency of the sensor structure matched the ODT vibrational modes, since the highest electric field intensity confined in the nanometric gaps occurs near the resonant frequencies of these nano-patch antenna structures. To further analyze these spectral features due to the analyte, we extracted the net molecular vibrational signals (i.e., the SEIRA signals) by performing a baseline subtraction and the results are plotted in FIG. 52(b). Individual spectral features associated with the aforementioned four ODT vibrational modes, highlighted by the colored regions in FIG. 52(b), can be clearly observed in these spectra. The maximum molecular vibrational signal achieved was approximately 10%, which was obtained from the sensor with 400 nm wide gold nanostrips, as its resonance matched the ODT vibrational modes the best. Such a sensing performance is superior to those of the previous demonstrations of SEIRA sensing of SAM ODT. In addition to the large field enhancement in the nanometric gaps and the high spatial overlap between the monolayer ODT and the enhanced field thanks to the liquid gallium ground plane, the geometry of the elongated nanostrips also contributes to improving the SEIRA sensing performance as it makes efficient use of the device surface area. The spectral features associated with the ODT vibrational modes generally exhibited asymmetric Fano-like line shapes, which is a result of the interference between the broadband resonance of the nano-patch antenna structure and the relatively narrow vibrational modes of ODT.

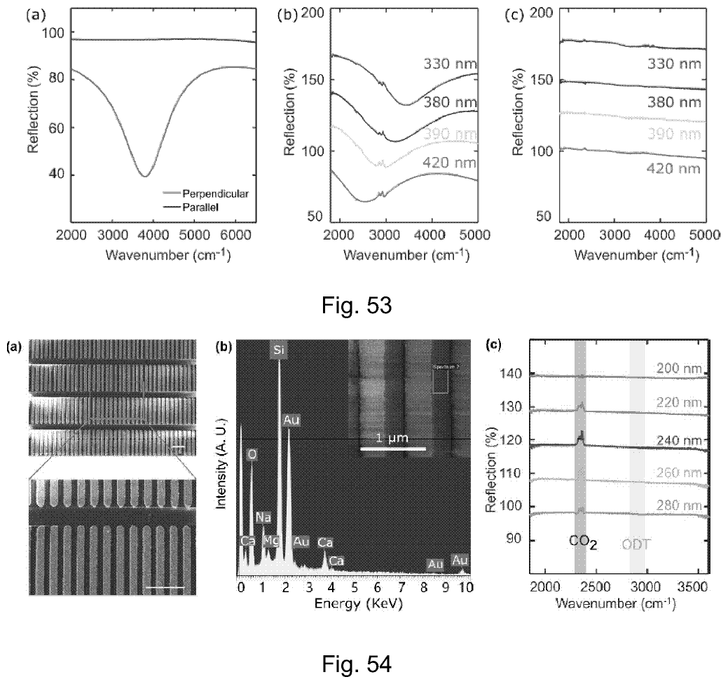

To consider effect of the light polarization, we also simulated and experimentally characterized the reflection spectra of the devices with the incident light linearly polarized parallel to the gold nanostrips, and the corresponding results are shown in FIG. 53. Since these nano-patch antennas do not have resonant modes in the spectral region of interest for this polarization of incident light (see FIG. 53(a)), as expected, no significant SEIRA signal was observed (see FIG. 53(c)).

After the optical measurement, the liquid gallium can be conveniently removed from the sensor surface by simply lifting the sensor chip up. As the surface tension of liquid gallium is relatively large (around 708 mN/m), liquid gallium generally does not wet with organic materials and hence can be easily removed from the thin analyte film without leaving residual. We imaged the sensor chip surface after removing the liquid gallium with a scanning electron microscope (SEM) (see FIG. 54(a)). The gold nanostrips appeared to be the same as before they were contacted with liquid gallium, and there was no evident gallium residual on the chip surface (see the zoomed-in image). We also characterized the sensor surface with energy-dispersive X-ray spectroscopy (EDS) (see FIG. 54(b)) to further examine whether there was any gallium residual. No gallium peak was found in the EDS spectra, which indicated that the sample was not contaminated with Ga. Removing the liquid gallium without residual allows for other subsequent analysis of the analytes and makes our sensor chips reusable (since many types of analytes can also be thoroughly removed), which can further reduce the cost of the various potential sensing applications enabled by such devices.

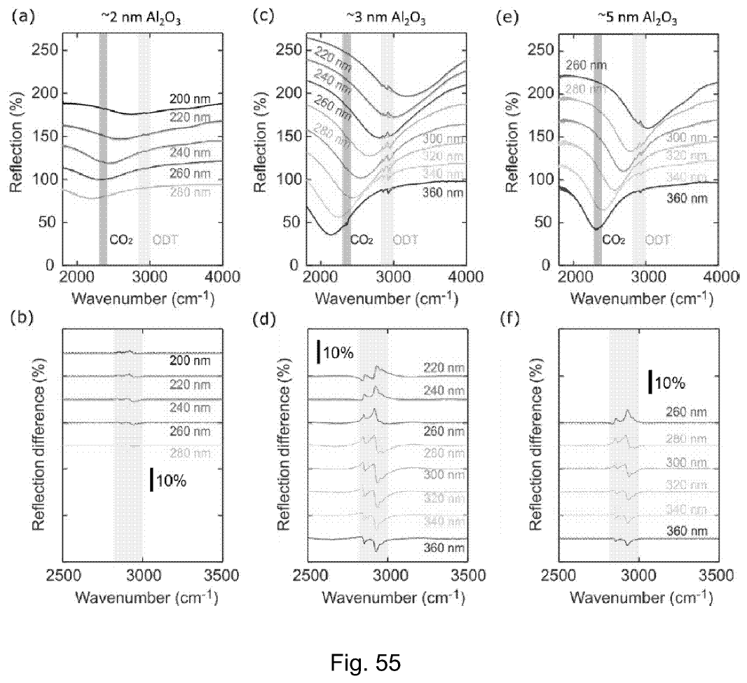

To compare the sensing performance of our liquid-gallium-based nano-patch antenna sensors to similar devices with a conventional ground plane made of noble metals, we fabricated several reference devices with a deposited gold or silver film as the ground plane. We found that when the gold or silver film was deposited directly on the SAM ODT using an electron-beam evaporator, neither the expected nano-patch antenna resonance nor the ODT absorption peaks were observed in the measured reflection spectra (see FIG. 55(c)). Note that the same ODT solution and SAM ODT preparation procedure were used for all the samples, and our repeated tests confirmed that the sample preparation procedure can reliably form SAM ODT that completely covers gold surface. Therefore, the quality of the SAM ODT on all the devices should be similar before either the liquid gallium or the deposited metal or dielectric film was introduced.

The results in FIG. 55(c) suggest that the SAM ODT was likely damaged during the metal deposition and consequently the nano-patch antenna structures were not successfully formed, because any direct contact between a gold nanostrip and the metallic ground plane establishes a “short circuit” that may severely quench the nano-patch antenna resonance. This issue associated with the metal deposition further demonstrates the advantage of using liquid gallium to form the ground plane.

To protect the SAM ODT from the metal deposition process, we prepared additional reference devices and deposited a thin aluminum oxide layer on the SAM ODT with atomic layer deposition (at 80° C.), before depositing the gold ground plane with the electron-beam evaporator. The reflection spectra of the best reference devices (with ˜3 nm aluminum oxide) are plotted in FIG. 55(c), which exhibit both the nano-patch antenna resonances and the SEIRA signals associated with the ODT vibrational modes. The net SEIRA signals due to the ODT vibrational modes are extracted and plotted in FIG. 55(d). Compared to the results from the liquid-gallium-based sensors, the maximum SEIRA signal obtained with these reference sensors is slightly lower (around 9%), and the signals associated with the CH₃-vibrational modes are evidently weaker and not clearly resolved. Since the CH₃-group is located at the end of the carbon-chain in an ODT molecule and constitutes the top surface of the SAM ODT, the weaker signals associated with the CH₃-vibrational modes suggest that the SAM ODT may still be affected by the depositions of the aluminum oxide film and/or the metal film. In addition, the reference devices with a thinner oxide film showed worse sensing performance (see FIG. 55(a)), likely due to insufficient protection of the SAM ODT. On the other hand, the reference devices with a thicker oxide film also showed lower SEIRA sensing performance, likely owing to the reduced field enhancement in the gap (see FIG. 55(e)). Although adding the oxide layer can protect the analytes, it inevitably increases the gap size of the nano-patch antenna, which in turn decreases the field enhancement and reduces the sensor performance. In comparison, our liquid-gallium-based sensor structures not only allow for simple and cost-effective device fabrication, but also lead to superior field enhancement and sensitivity performance.

### 5.3.3 Measured Spectrum for Device Coating with PMMA Thin Films

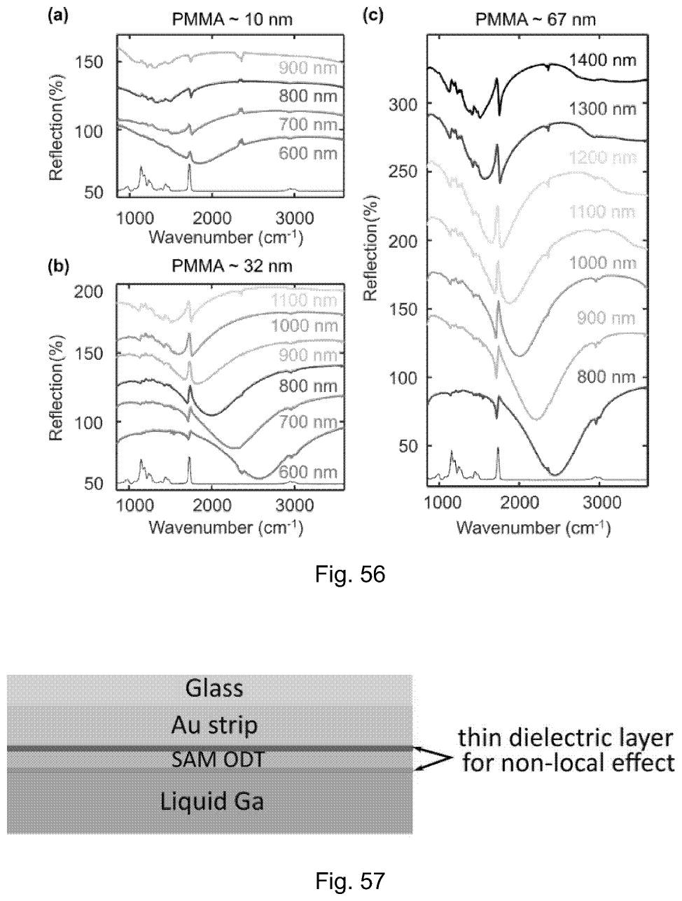

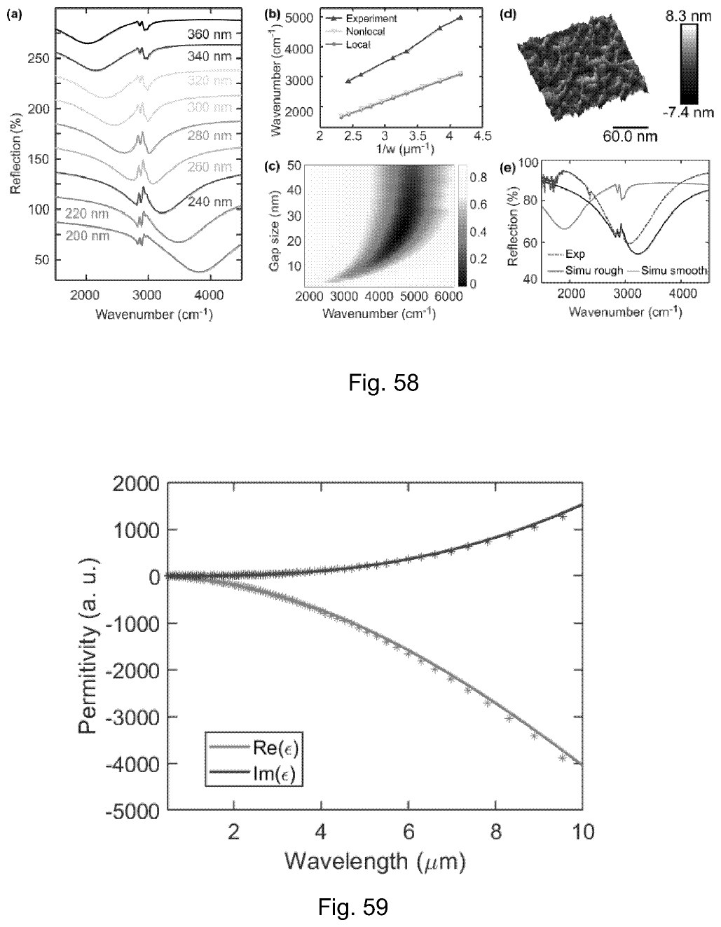

In principle, the demonstrated nano-patch antenna sensors employing a liquid gallium ground plane can operate in a broad infrared spectral range and for a wide range of analyte film thickness. A variety of chemical and biological substances have molecular vibrational modes in the spectral range of 1000 cm^{-1˜2000} cm⁻¹, such as the amide bands of proteins, which are of crucial importance for various biosensing applications. Therefore, we implemented another batch of sensors to target the spectral region around 1500 cm⁻¹. CaF₂ substrates were used for these devices, because CaF₂ is transparent in a much wider infrared spectral range than glass. We chose PMMA as the model analyte to demonstrate the sensor performance, because PMMA has multiple vibrational modes in the spectral range from 1000 cm⁻¹ to 2000 cm⁻¹. PMMA dissolved in anisole with concentrations of 0.5%, 1%, and 2% were spin-coated on the surface of the gold strips, and the measured film thicknesses were approximately 10 nm, 32 nm, and 67 nm, respectively. The sensor chips were then placed on liquid gallium to form the complete nano-patch antenna structures, before their reflection spectra were measured (see FIG. 56). Again, resonant reflection dips of these nano-patch antennas can be clearly observed in the measured reflection spectra, and the resonance shifts to lower frequency as the gold strip width increases. With the increase of the PMMA film thickness, the resonant reflection dip also becomes larger, which is consistent with the simulation results in FIG. 58(c). The Fano-like spectral feature associated with the molecular vibrational mode of PMMA at 1750 cm 1 is particularly evident in all the reflection spectra, which becomes stronger with the increase of the PMMA thickness. Similar to the ODT sensing results, the SEIRA signals associated with the PMMA are larger when the sensor resonant frequency matches well with the frequencies of the PMMA vibrational modes. To further analyze the sensitivity of the sensors with different PMMA film thicknesses, we extracted the net molecular vibrational signals associated with the vibrational mode at 1750 cm⁻¹ (C═O stretching) after a baseline subtraction. The maximum molecular vibrational signals of 7.4%, 16.6%, and 28.1% were obtained from the sensors coated with 10 nm, 32 nm, and 67 nm thick PMMA films, respectively.

### 5.4 Comparison Between Simulation and Experimental Results

### 5.4.1 Numerical Simulation Configuration

Numerical simulations were conducted by using a commercial software (Lumerical FDTD Solutions) which is based on the FDTD method. Two-dimensional simulations were performed with periodic boundary conditions in the device plane and perfectly matched layer (PML) boundary conditions along the light propagation direction. The relative permittivity of ODT and liquid gallium were derived from the experimental results. The relative permittivity of gold was obtained from tabulated data. The thickness of the SAM ODT is set to 2.4 nm. The plane wave source and the reflection monitor were positioned at 5 μm and 7 μm above the gold nanostrip, respectively. The mesh size in the nanometric gap (i.e., the SAM ODT layer) was 0.3 nm.

The non-local effect of metals may not be neglected when the plasmon dimensions become comparable to the Thomas-Fermi screening length. To take into account the non-local effect owing to the nanometric gaps in our nano-patch antenna structures, we employed an effective local model in the simulation by replacing the non-local metal with a composite structure having a thin dielectric layer on top of a local metal. FIG. 57 illustrates the schematic of this equivalent local model which effectively takes into account the non-local effect. This equivalent model is valid as long as (i) the thickness Δd of the thin dielectric layer is much smaller than the metal's skin depth, and (ii) the permittivity of this dielectric layer & is proportional to Δd with the relation:

$$\frac{{ε}_{t}}{Δ d} = \frac{{ε}_{m} {ε}_{b} {q}_{L}}{{ε}_{m} - {ε}_{b}} \qquad ( 5.1 )$$

where εₘ is the permittivity of the metal, ε_{b} is the permittivity of the background, and q_{L} is the longitudinal plasmon normal wave vector. In our simulation, Δd was set to 0.1 nm and ε_{b}=2.1 was used (i.e., the non-dispersive component of the ODT relative permittivity). The corresponding relative permittivity of the thin dielectric layer on gold and liquid gallium were calculated to be 1.54 and 2.02, respectively.

### 5.4.2 Simulation Results

FIG. 58(a) shows the simulated reflection spectra of the sensors with the gold nanostrip width ranging from 200 nm to 380 nm. Compared with the experimental results, the simulated reflection spectra have similar spectral features but also exhibit two important discrepancies. First, for the same nanostrip width, the simulated reflection spectrum of a nano-patch antenna array shows a significantly lower resonant frequency than that obtained from the experiment. Second, the simulated molecular vibrational signals caused by the SAM ODT can reach ˜20%, which is about twice as large as those extracted from the experimental spectra. Considering that the non-local effect may play a role in determining the spectral responses of our sensor structures with nanometric gaps, we performed simulation to quantify the influence of the non-local effect. FIG. 58(b) compares the simulated nano-patch antenna resonant frequency, with or without considering the non-local effect, to the experimental values (plotted as functions of the inverse of the nanostrip width). Although the simulated resonant frequencies show a small blue-shift when the non-local effect is considered, they are still significantly below the corresponding experimental values. Therefore, the non-local effect cannot explain this discrepancy in resonant frequency. Besides the non-local effect, the gap size of the nano-patch antenna may also significantly influence the resonant frequency. As shown in FIG. 58(c), we simulated the reflection spectra of a nano-patch antenna structure with varied gap sizes (the nanostrip width was fixed at 380 nm). When the gap size is relatively large (e.g., larger than 30 nm), the resonant frequency of the nano-patch antenna shows a relatively weak dependence on the gap size. However, when the gap size becomes smaller, the resonant frequency of the nano-patch antenna exhibits a strong dependence on the gap size, especially when the gap size is below 10 nm. The reflection dip at the resonance also reduces with the decrease of the gap size, as the system deviates further from the critical coupling condition. Considering the surface roughness of the gold nanostrips and the relatively large surface tension of liquid gallium, we surmise that the liquid gallium did not form a perfectly conformal contact with the SAM ODT coated on the gold nanostrips. Instead, owing to the surface roughness of the gold nanostrips, there were randomly distributed small gaps between the liquid gallium and the SAM ODT, which effectively increased the average gap size of the nano-patch antennas and hence led to a significant increase of the resonant frequency.

We characterized the surface morphology of the gold nanostrips using atomic force microscopy (AFM) and found that the surface indeed has randomly distributed protrusions that are several nm in height (see FIG. 58(d)). We further conducted simulations which took into account such surface roughness of the gold nanostrips as well as the corresponding undulating surface profile of the liquid gallium. In the simulation models, the liquid gallium only contacted the protrusions of the SAM ODT-covered rough gold nanostrip surface and therefore small air gaps were formed in between the protrusions (see Section D for details). FIG. 58(e) compares the experimental spectrum with the simulated spectra obtained with and without taking into account the surface roughness of the gold nanostrips. Indeed, with the surface roughness incorporated in the model, the simulated reflection spectrum significantly shifts to a higher frequency and matches the experimental result well. Since a larger effective gap size results in lower field confinement and enhancement, which in turn reduces the sensor sensitivity, our experimentally demonstrated sensor performance may be further improved significantly by reducing the surface roughness of the gold nanostrips. This can be achieved, for example, by optimizing the metal deposition process or employing the template stripping method.

### 5.5 Discussion and Conclusion

Although we utilized SAM ODT as the model analyte to achieve a proof-of-concept demonstration of liquid-gallium-based nanophotonic SEIRA sensors targeting the mid-infrared spectral region around 3000 cm⁻¹, this unconventional device architecture and design strategy can be applied to SEIRA sensors targeting other analytes and other infrared spectral regions, since liquid gallium exhibits the properties of a good metal across a broad infrared spectral range (FIG. 48(c)). To demonstrate the spectral versatility of such liquid-gallium-based nanophotonic sensors, we also implemented another batch of sensors by simply scaling up the structure dimensions to target the spectral region around 1500 cm⁻¹, which is of crucial importance for various biosensing applications because it contains the amide bands of proteins. In this case, we chose poly(methyl methacrylate) (PMMA) as the model analyte to demonstrate the sensor performance, as PMMA has multiple vibrational modes in this spectral region and can be conveniently spin-coated on the surface of the gold strips. These sensors also demonstrated high sensing performance for PMMA thin films of varied thicknesses, even though the device structures were not systematically optimized. In addition to gallium, several other pure metals are also in the liquid phase at moderate temperatures, such as the alkali metals (e.g., potassium, rubidium) and mercury, which in principle can be used for realizing liquid-metal-based nanophotonic structures and devices. However, alkali metals have high chemical reactivity and high costs, whereas mercury has severe toxicity, and therefore they are not suitable for sensing applications. In this perspective, liquid gallium has a unique advantage. Furthermore, several gallium-based alloys, such as the eutectic gallium-indium and Galinstan, have even lower melting points and are also cost-effective, making them candidate liquid metals for realizing the demonstrated nanophotonic SEIRA sensors which are suitable for point-of-care applications. In conclusion, we experimentally demonstrated high-performance SEIRA sensors based on nano-patch antenna structures which employ liquid gallium as the ground plane. Thin analyte films can be conveniently coated on patterned gold nanostrips and then covered by liquid gallium to form the complete nano-patch antenna structures with nanometric gaps, in which ultra-high field intensity is confined. Therefore, this approach achieves both sensing hot spots with exceedingly large field enhancement and efficient delivery of analytes into the hot spots without affecting the properties of the analytes, leading to high-performance SEIRA sensing of nanometric analyte films. A simple and effective way was developed to cover the sensor chips with liquid gallium for optical measurement and to remove the liquid gallium afterwards, which allows the sensor chips to be reused. Our liquid-gallium-based SEIRA sensors produced around 10% molecular vibrational signals near 2900 cm⁻¹ from a SAM ODT, which is a performance superior to those of the previous demonstrations of SEIRA sensing of SAM ODT in the literature and our reference sensors employing a noble metal ground plane. We also demonstrated sensors which targeted the spectral region near 1500 cm⁻¹ and achieved high-performance sensing of nanometric PMMA films. In-depth comparison and analysis of our experimental and simulation results further suggest that the sensing performance of our liquid-gallium-based sensors can be further improved by reducing the surface roughness of the patterned gold nanostrips, which currently limits the minimum effective gap size of the nano-patch antenna structures. The demonstrated cost-effective and reproducible method of employing liquid metals to construct nanophotonic structures with ultra-high field confinement and enhancement provides a new platform for various molecular sensing applications, and may also be used for enhancing other types of light-matter interactions for a broad range of applications, such as index sensing, surface-enhanced Raman scattering sensing, nonlinear optics and cavity quantum electrodynamics.

### 7. Conclusion

Photonic nanostructures can strongly interact with the molecules adsorbed on the surface by confining strong electromagnetic fields on the nanometer scale and hence provide the enhanced vibrational signal in the SERS and SEIRA sensing applications. The signal enhancement can be well improved by tailoring the optical response with optimized geometry design and the choice of materials. In this disclosure, advanced photonics designs for realizing the high-sensitivity SEIRA and SERS sensors are investigated. Three different main strategies are applied to optimize photonics structures for higher sensitivity: a cost-effective self-aligned method to fabricate the nanometric gap given in section 3, a passive trapping method to realize the concentration and trapping of analyte molecules in solution into the sensor hot spot in section 4, and a uniform coverage of liquid gallium on the analyte film to form nanometric hot spots.

Graphene plasmonic is a good platform for the thin layer SEIRA sensing due to its high field confinement but it is hard to excite and shows a low extinction response, which requires longer measurement time and much sensitive instrument to obtain a spectrum with a good signal-to-noise ratio. The hybrid structures of graphene antidots and gold cores separated with uniform nanometric size gaps were exploited for realizing higher electric field enhancement as well as the extinction response. Employed the same fabrication process as the patterned graphene device, the hybrid device was fabricated by introducing the additional metal deposition step for the metal core immediately after etching the graphene. This self-aligned method can realize the controllable gap size around 50 nm to 120 nm without significantly increasing the fabrication cost. Thanks to the lightning rod effect, the nanogap can confine the stronger electric field. The experimental results showed that the GSPs in the hybrid devices had an almost 3 times stronger extinction response compared with the devices only with graphene patterns.

On the other aspect, guiding more molecules into the hot spot with high field enhancement on the sensors will also be beneficial for improving SEIRA sensor performance. The presently disclosed nanophotonic SEIRA sensors are designed not only with large-field enhancement confined in the hot spot but also with the functionality of concentrating and trapping analyte molecules in an evaporating solution in hot spots, hence leading to significantly improved SEIRA sensing performance. Different from the active trapping method, the trapping mechanism requires no external energy source (i.e., passive) and can be applied to various molecular species and nano-particles, such as liposomes. From the proof-concept experiment, the designed SEIRA sensor can derive the vibrational information from target analyte molecules L-proline and D-glucose with a mass down to 1 pg by combing the proposed concentrating and trapping procedure, which corresponds to significantly less than one monolayer of analyte molecules when averaged over the entire measurement area.

The decrease of the gap size will provide a higher electric field and hence higher vibrational signal enhancement. The ideal hot spot size for the SERS and SEIRA sensors is exactly the same as the size of analyte molecules and hence the hot spot of the sensor can provide maximum electric field enhancement and vibrational signal enhancement. However, the smaller gap will lead to the increasingly hard to deliver the analyte into the gap. Utilizing the liquid gallium as the ground plane, the proposed nano-patch antenna can fill the nanometric gaps with the ultrathin analyte film to realize high-performance SEIRA sensing. A simple and effective way was developed to make the liquid gallium conformally covered on the analyte film adsorbed the gold nanoribbons, leading to the formation of the nanometric hot spot. The formation of this nanometric size hotspot can realize exceedingly large field enhancement and avoid the limitation of delivery of analytes into the hot spots without affecting the properties of the analytes, leading to high-performance SEIRA sensing of nano-metric analyte films. These SEIRA sensors experimentally exhibited state-of-the-art sensing performance for nanometric analyte thin films, such as monolayer ODT as well as PMMA thin film. The sensing performance of our current devices can be further improved by a smooth surface of the patterned gold nanostrips due to the smaller effective gap size in the nano-patch antenna structures, which are verified by the in-depth comparison and analysis of our experimental and simulation results.

Similar strategies of coating liquid gallium on analyte film can be applied to the SERS sensors. Different from our gallium based SEIRA sensor which involved the gold nanoribbons on the substrate, SERS sensors are based on cost-effective RTA annealing produced gold nanoparticle substrate. After conformally covering the analyte film on the gold nano-particles, the formed nanometric gaps support a highly enhanced electric field and hence boost the Raman signal of the analyte in the gaps. The experimental results show that the Raman sensing signal with gold nanoparticles for sensing SAM BPT can be increased by almost one order of magnitude after introducing the liquid gallium, which is consistent with simulation results of higher electric field enhancement due to the covering of liquid gallium. In addition, the spatial uniformity of the measured Raman signals was found to be improved.

### A. Drude Model for the Metal

The optical response of noble metals by illuminating a light depends on the frequency, which can be described by a complex dielectric function. The origin of these optical properties mainly comes from the free electrons in the conduction band. Drude used the kinetic theory of gases applied to the gas of electrons moving on a fixed background of “ions” while ignoring the interaction between the electrons. The electron movement can be described by the equation:

$${m}_{e} \frac{{\partial }^{2} r}{\partial {t}^{2}} + {m}_{e} Γ \frac{\partial r}{\partial t} = {eE}_{0} {e}^{- i ω t} \qquad ( A \text{.1} )$$

where e and mₑ are the charge and effective mass of the free electrons, and E₀e^{-iωt} is the electric field of the applied light. Γ is the damping rate of electrons Solving the equation, we get the displacement of the electron:

$$r = \frac{e}{{m}_{e}} \frac{1}{i ωΓ - {ω}^{2}} {E}_{0} {e}^{- i ω t} \qquad ( A \text{.2} )$$

By summing the dipole moments of each electron according to p=er, we can obtain the total dipole moments with the presence of an electric field as:

$$P ( ω ) = np = enr \qquad ( A \text{.3} )$$

Solving using the equations, and the relation D(ω)=ε₀ε(ω)E(ω)=ε₀E(ω)+P(ω) yields:

$$ε ( ω ) = 1 + \frac{{ne}^{2}}{m} \frac{1}{i ωΓ - {ω}^{2}} = 1 - \frac{{ω}_{p}^{2}}{{ω}^{2} + i Γω} \qquad ( A \text{.4} )$$

with volume plasma frequency ωₚ=\sqrt{nₑ²/(mₑε₀)}. The permittivity of metals such as gold, silver, and aluminum can be modeled with the Drude model. Here we use gold as an example. FIG. 59 gives the permittivity of the gold with the experimental data and the corresponding Drude model with the plasma frequency of 8.45 eV and the damping rate of 0.295 eV. We can see that the real part of the permittivity is negative, which is the common property of the metals. One obvious consequence of this negative real permittivity prevents the light from penetrating into metal.
B. Extracting the Relative Permittivity of Proline from Experimental Data

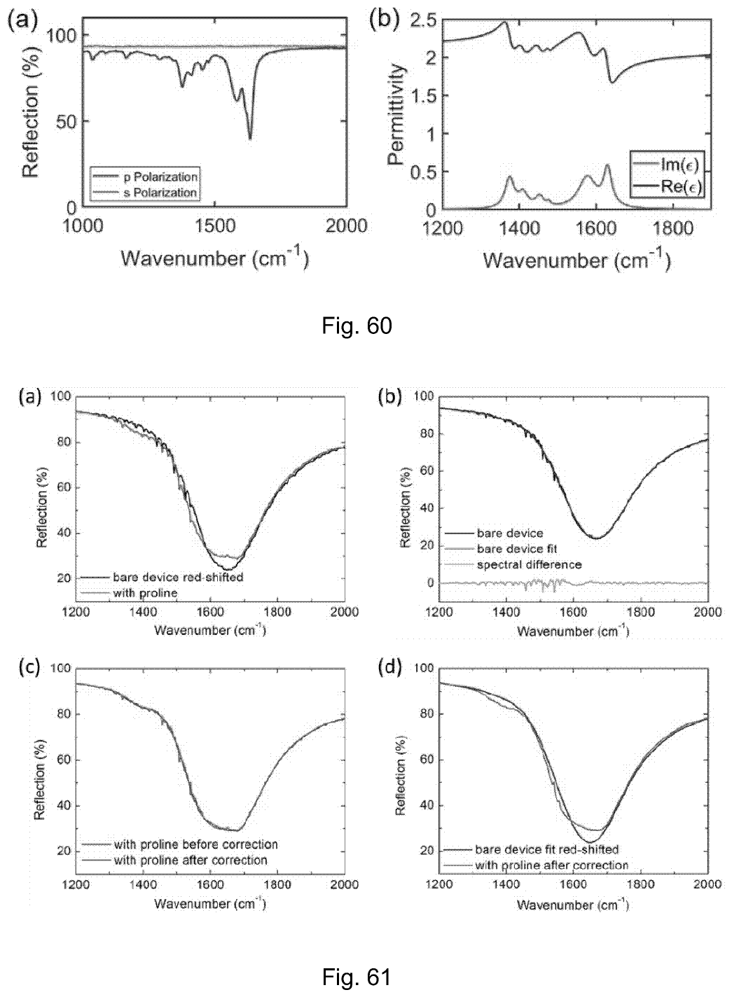

We conducted IR reflection absorption spectroscopy measurement of a thin proline film on a gold mirror, and extracted the relative permittivity function of proline from the measured spectrum. The reflection spectrum R (θ) of a thin film (i.e., t<<λ) on a gold mirror (close to a perfect mirror in IR) can be approximated by the formula:

$$\frac{R ( θ )}{{R}_{0} ( θ )} \approx 1 - 4 t \frac{ω}{c} \frac{{sin}^{2} ( θ )}{{cos}^{2} ( θ )} lm \{ - \frac{1}{{ε}_{p}} \} \qquad ( B \text{.1} )$$

where θ is the angle of incidence, R₀(θ)≈1 is the reflection spectrum of the bare gold mirror (close to a perfect mirror in IR), t is the thickness of the thin film and εₚ is the complex relative permittivity of the thin film. In our experiment, the proline thin film was sublimated on the gold mirror by heating proline powder at 150° C. in vacuum. The thickness of the thin film is around 150 nm to 200 nm, which was measured using a profilometer. The reflection spectra were measured for both p-polarization and s-polarization of the incident light at 80° incident angle, which are plotted in FIG. 60. Six absorption peaks of different strengths in the spectral region from 1300 cm⁻¹ to 1700 cm⁻¹ can be clearly observed in the reflection spectrum of the p-polarization, which are listed in Table B.1. We modeled the relative permittivity function of proline using the Lorentz model and taking into account these six absorption lines, which is expressed by the following formula:

$${ε}_{p} ( ω ) = {ε}_{∞} + {\sum }_{j = 1}^{6} \frac{{S}_{j} {ω}_{0 , j}^{2}}{{ω}_{0 , j}^{2} - {ω}^{2} - i ω {γ}_{j}} \qquad ( B \text{.2} )$$

where ω_{0,j}, Sⱼ,γⱼ are the angular frequency, the oscillator strength, and the damping rate of the j-th absorption line, respectively. Assuming the non-dispersive component of the relative permittivity to be ε_{∞}=2.1, the Lorentz model parameters Sⱼ and γⱼ were extracted by fitting equation B.2 to the experimental data, and the fitting results are listed in Table B.1. The calculated relative permittivity function of proline using the fit parameters is plotted in FIG. 60(b).

TABLE B.1
Parameters for the proline relative permittivity model

| | | | | | | |
|---|---|---|---|---|---|---|
| ω0, j (cm⁻¹) | 1375 | 1410 | 1453 | 1477 | 1577 | 1630 |
| γⱼ (cm⁻¹) | 28 | 30 | 26 | 12 | 55 | 26 |
| Sⱼ | 0.0081 | 0.004 | 0.0026 | 0.00045 | 0.0145 | 0.008 |

### C. Extracting the Differential Reflection Spectrum

When the analyte solution concentration is relatively low, the broad resonance in the reflection spectrum of a device undergoes a moderate red-shift while preserving its line shape (see for example FIG. 44(d) upper panel). Therefore, by applying a red shift to the reflection spectrum of the bare device accordingly, we can overlap the broad resonance in the reflection spectrum of the bare device to that of the device with a trace amount of analyte precipitate. The calculated difference between such two spectra is the differential spectrum which is mainly due to the analyte molecules' absorption lines (see for example FIG. 44(d) lower panel). However, this methods of extracting the differential spectrum does not apply to relatively high concentration analyte solutions, because in those cases the resonance in the reflection spectrum not only undergoes a larger red-shift, but also changes its line shape significantly (see for example FIG. 44(b)-(c)). Without further data processing, the extracted differential spectrum also contains significant interfering spectral features due to water vapor absorption lines. In order to remove or reduce these interfering spectral features due to the water vapor absorption lines, we performed the following data processing steps. We first obtained a smooth baseline fit

$$( {R}_{bare}^{′} )$$

to the measured reflection spectrum (R_{bare}) of the bare device using a high-order polynomial function, and then calculated the difference between R_{bare} and

$${R}_{bare}^{′} ,$$

i.e.,

$$δ {R}_{bare} = {R}_{bare} - {R}_{bare}^{′} .$$

This extracted δR_{bare} mainly includes the narrow spectral features due to the water vapor absorption lines. Since the measured reflection spectrum of the same device with analyte precipitate (Rₛₑₙₛ) contains similar spectral features due to the water absorption lines, we subtracted δR_{bare} from Rₛₑₙₛ to obtain a reflection spectrum corrected for these water absorption lines.

$${R}_{sens}^{′} = {R}_{sens} - δ {R}_{bare} .$$

Finally, we calculated the difference between

$${R}_{sens}^{′}$$

and the red-shifted

$${R}_{bare}^{′}$$

to obtain the differential spectrum which is mainly due to the analyte absorption lines, with much weaker spectral features associated with the water absorption lines (see for example FIG. 44(d) lower panel and FIG. 45(b)).

FIG. 61 shows the spectra of the intermediate steps of the procedure for extracting the differential spectra (described in the “Materials and methods” section above) in FIG. 44(d) lower panel. Specifically, FIG. 61(a) shows the red-shifted reflection spectrum of the bare device which overlaps well with the reflection spectrum of the same device with the proline precipitate from the 0.2 μg/mL solution, whereas their difference corresponds to the red differential spectrum plotted in FIG. 44(d) lower panel; FIG. 61(b) shows the polynomial fit

$$( {R}_{bare}^{′} )$$

to the reflection spectrum of the bare device (R_{bare}) across the spectral range between 1200 cm⁻¹ and 2000 cm−1, and the difference between the measured and the fit spectra, i.e.,

$$δ {R}_{bare} = {R}_{bare} - {R}_{bare}^{′} ,$$

which mainly is made up of spectral features due to the wayer absorption lines; FIG. 61(c) shows the reflection spectrum of the device with the proline precipitate (Rₛₑₙₛ) and the corrected spectrum after subtracting δR_{bare}

$$( i . e . , {R}_{sens}^{′} = {R}_{sens} - δ {R}_{bare} )$$

to remove/reduce the interfering water absorption lines; FIG. 61(d) shows the corrected reflection spectrum of the device with the proline precipitate

$$( {R}_{sens}^{′} )$$

and the red-shifted

$${R}_{bare}^{′} ,$$

and their difference corresponds to the blue differential spectrum plotted in FIG. 44(d) lower panel.

### D. Simulations Considering the Surface Roughness of the Gold Nanostrips

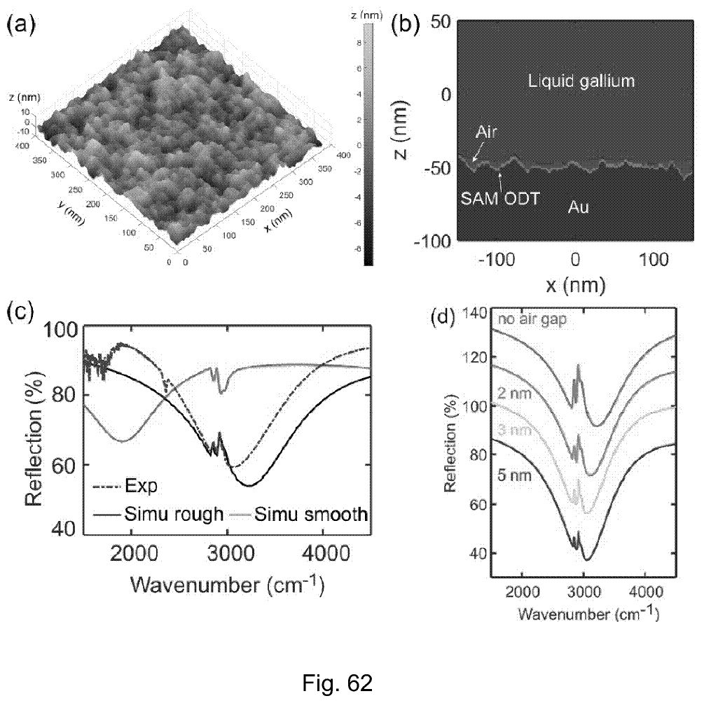

As shown section 5.4.2 above, the ODT-coated gold nanostrips have a surface roughness of several nm, which effectively leads to a larger gap size of the nano-patch antennas employing a liquid gallium ground plane, and hence causes a blue shift of their resonant frequencies. In our simulations, we modeled such surface roughness by generating random rough surfaces with the statistical parameters extracted from the measured surface morphology of the ODT-coated gold nanostrips, as shown in the example in FIG. 62(a). We assumed that the roughness height of the gold surface follows a Gaussian distribution, and its autocorrelation function is exponential. As mentioned above, the surface morphology of the ODT-coated gold nanostrips was characterized using AFM, and an example is given in FIG. 58(d) of the main text. The roughness height rms value and the roughness correlation length were found to be about 2.4 nm and 12 nm, respectively, which were used in our model to generate random rough surfaces based on an open-source code. The monolayer ODT was modeled to strictly follow the profile of the gold surface, whereas the surface of the liquid gallium was modeled as a smoothened version of the ODT/gold surface which has direct contacts with the ODT layer at the peaks (i.e., the local maxima), as illustrated in the exemplary cross-sectional view in FIG. 62(b). The simulated spectra indeed show good agreement with the experimental results, in terms of both the resonant frequencies of the nano-patch antennas and the SEIRA signals associated with the SAM ODT, as can be seen in FIG. 62(c). These results suggest that the surface roughness is likely the main contributing factor which caused the significant blue shift of the resonant frequencies of the nano-patch antennas (compared to those of the ideal structures with ˜2.4 nm gap size).

As the random surface roughness of the gold nanostrips effectively increases the average gap size of the nano-patch antennas, we can also employ a thin air gap between the SAM ODT and the liquid gallium surface to model such an averaged effect of the surface roughness. Several structures with varied air gap sizes were simulated, and their resonant frequencies were tuned to overlap with the ODT vibrational modes by adjusting the widths of the gold nanostrips. These simulated spectra are shown in FIG. 62(d). Indeed, the SEIRA signals associated with the SAM ODT decrease evidently with the increase of the gap size, which suggests that a smoother gold surface (i.e., smaller gap size) should lead to higher sensing performance.

The steps of the methods described in the various examples disclosed herein are sufficient to carry out the methods of the present disclosure. Thus, in various examples, a method consists essentially of a combination of the steps of the methods disclosed herein. In various other examples, a method consists of such steps. All examples and embodiments provided herein are intended to be non-limiting unless otherwise specified.

Although the present disclosure has been described with respect to one or more particular embodiments, it will be understood that other embodiments of the present disclosure may be made without departing from the spirit and scope of the present disclosure.

We claim:

1. A method of preparing a sample for Raman spectroscopy, the method comprising:
disposing a plurality of metal nanoparticles on a first surface of a substrate;
depositing an analyte of interest on the metal nanoparticles;
coating at least a portion of the first surface of the substrate in a liquid metal such that the plurality of metal nanoparticles and analyte of interest are encapsulated between the first surface of the substrate and the liquid metal.

2. The method of claim 1, wherein the plurality of metal nanoparticles are disposed on the first surface of the substrate by:
depositing a thin film of the metal onto the first surface of the substrate; and
annealing the thin film to form the plurality of metal nanoparticles

3. The method of claim 1, wherein the plurality of metal nanoparticles are formed by lithography, metal deposition, or lift-off.

4. The method of claim 1, wherein the plurality of metal nanoparticles are silver or gold.

5. The method of claim 1, wherein the liquid metal is gallium or liquid gallium-indium.

6. The method of claim 1, wherein the substrate comprises glass or fused silica.

7. The method of claim 1, wherein the analyte of interest is adsorbed on the plurality of metal nanoparticles.

8. The method of claim 1, wherein the plurality of metal nanoparticles comprises nanoparticles of varying diameters.

9. The method of claim 1, wherein a diameter of each of the plurality of metal nanoparticles is within 10% of a diameter of the other metal nanoparticles of the plurality of metal nanoparticles.

10. The method of claim 1, wherein the average diameter of the plurality of metal nanoparticles is between 20 nm and 30 nm, inclusive.

11. The method of claim 1, wherein the first surface of the substrate is coated in liquid metal by placing the first surface of the substrate in liquid metal.

12. The method of claim 1, wherein each metal nanoparticle of the plurality of metal nanoparticles has a diameter in the range of 10 nm to 100 nm, inclusive.

13. The method of claim 1, wherein each metal nanoparticle of the plurality of metal nanoparticles has a diameter in the range of 20 nm to 80 nm, inclusive.

14. The method of claim 1, wherein one or more of the metal nanoparticles of the plurality of metal nanoparticles has a diameter less than 10 nm.

15. The method of claim 1, wherein the metal nanoparticles of the plurality of metal nanoparticles are separated by distances ranging from 2 nm to 100 nm, inclusive.

16. A sensor for Raman spectroscopy made according to the method of any one of claims 1-15.

17. A sensor for Raman spectroscopy, comprising:
a substrate;
a plurality of metal nanoparticles disposed on at least a portion of a first surface of the substrate;
an analyte of interest adsorbed on the plurality of metal nanoparticles; and
a liquid metal coating on at least a portion of the first surface of the substrate thereby encapsulating the plurality of metal nanoparticles and analyte of interest between the substrate and the liquid metal.

18. The sensor of claim 17, wherein the plurality of metal nanoparticles are silver or gold.

19. The sensor of claim 17, wherein the liquid metal is gallium or liquid gallium-indium.

20. The sensor of claim 17, wherein the substrate comprises glass or fused silica.

21. The sensor of claim 17, wherein the analyte of interest is adsorbed on the plurality of metal nanoparticles.

22. The sensor of claim 17, wherein the plurality of metal nanoparticles comprises nanoparticles of varying diameters.

23. The sensor of claim 17, wherein a diameter of each of the plurality of metal nanoparticles is within 10% of a diameter of the other metal nanoparticles of the plurality of metal nanoparticles.

24. The sensor of claim 17, wherein the average diameter of the plurality of metal nanoparticles is between 20 nm and 30 nm, inclusive.

25. The sensor of claim 17, wherein the first surface of the substrate is coated in liquid metal by placing the first surface of the substrate in liquid metal.

26. The sensor of claim 17, wherein each metal nanoparticle of the plurality of metal nanoparticles has a diameter in the range of 10 nm to 100 nm, inclusive.

27. The sensor of claim 17, wherein each metal nanoparticle of the plurality of metal nanoparticles has a diameter in the range of 20 nm to 80 nm, inclusive.

28. The sensor of claim 17, wherein one or more of the metal nanoparticles of the plurality of metal nanoparticles has a diameter less than 10 nm.

29. The sensor of claim 17, wherein the metal nanoparticles of the plurality of metal nanoparticles are separated by distances ranging from 2 nm to 100 nm, inclusive.

30. A method of preparing a sample for Raman spectroscopy, the method comprising:
disposing a plurality of SERS-active nanoparticles on a first surface of a substrate;
depositing an analyte of interest on the SERS-active nanoparticles;
conformally coating at least a portion of the first surface of the substrate in a conductor such that the plurality of SERS-active nanoparticles and analyte of interest are encapsulated between the first surface of the substrate and the conductor.

31. The method of claim 1, wherein the plurality of SERS-active nanoparticles are disposed on the first surface of the substrate by:
depositing a thin film of a SERS-active material onto the first surface of the substrate; and
annealing the thin film to form the plurality of SERS-active nanoparticles

32. The method of claim 1, wherein the plurality of SERS-active nanoparticles are formed by lithography, metal deposition, or lift-off.

33. The method of claim 1, wherein the plurality of SERS-active nanoparticles are silver or gold.

34. The method of claim 30, wherein the conductor is a liquid.

35. The method of claim 30, wherein the conductor is a liquid metal.

36. The method of any one of claim 34 or 35, wherein the conductor solidifies after coating the first surface of the substrate.

37. The method of claim 30, wherein the conductor is liquid gallium or liquid gallium-indium.

38. The method of claim 30, wherein the substrate comprises glass or fused silica.

39. The method of claim 30, wherein the analyte of interest is adsorbed on the plurality of metal nanoparticles.

40. The method of claim 30, wherein the plurality of metal nanoparticles comprises nanoparticles of varying diameters.

41. The method of claim 30, wherein a diameter of each of the plurality of metal nanoparticles is within 10% of a diameter of the other metal nanoparticles of the plurality of metal nanoparticles.

42. The method of claim 30, wherein the average diameter of the plurality of metal nanoparticles is between 20 nm and 30 nm, inclusive.

43. The method of claim 30, wherein the first surface of the substrate is coated in liquid metal by placing the first surface of the substrate in liquid metal.

44. The method of claim 30, wherein each metal nanoparticle of the plurality of metal nanoparticles has a diameter in the range of 10 nm to 100 nm, inclusive.

45. The method of claim 30, wherein each metal nanoparticle of the plurality of metal nanoparticles has a diameter in the range of 20 nm to 80 nm, inclusive.

46. The method of claim 30, wherein one or more of the metal nanoparticles of the plurality of metal nanoparticles has a diameter less than 10 nm.

47. The method of claim 30, wherein the metal nanoparticles of the plurality of metal nanoparticles are separated by distances ranging from 2 nm to 100 nm, inclusive.

48. A sensor for Raman spectroscopy made according to the method of any one of claims 30-47.
